SEQUENCE LISTING (1 sequence)

SEQ ID NO: 1 - protein, 21 aa, synthetic construct
MGSSHHHHHHSAALEVLFQGP

(19) United States
(12) Patent Application Publication
(10) Pub. No.: US 2026/0217686 A1
(43) Pub. Date: Jul. 30, 2026

(54) N-Myristoyltransferase Inhibitors And Methods Of Use

(71) Applicant: Seattle Children's Hospital (d/b/a Seattle Children's Research Institute), Seattle, WA (US)

(72) Inventors: Alexis Kaushansky, Shoreline, WA (US); Bart Staker, Poulsbo, WA (US); Rachael Zigweid, Seattle, WA (US); Kamalakannan Vijayan, Thiruvananthapuram (IN); Morten Grøti, Göteborg (SE); Diego Rodriguez-Hernandez, Göteborg (SE)

(21) Appl. No.: 19/143,989

(22) PCT Filed: Dec. 26, 2023
(86) PCT No.: PCT/US2023/085886

§ 371 Date: Jun. 27, 2025

Related U.S. Application Data
(60) Provisional application No. 63/477,448, filed on Dec. 28, 2022.

(51) Int. Cl. C07D 403/12; A61K 31/415; A61K 31/4406; A61K 31/4439; A61K 31/496; A61K 31/506; A61P 33/06; C07D 213/30; C07D 213/65; C07D 231/12; C07D 401/12; C07D 401/14

(52) U.S. Cl. CPC C07D 403/12; A61K 31/415; A61K 31/4406; A61K 31/4439; A61K 31/496; A61K 31/506; A61P 33/06; C07D 213/30; C07D 213/65; C07D 231/12; C07D 401/12; C07D 401/14

(57) ABSTRACT

Provided herein are compounds of Formula (I), which are useful as N-myristoyltransferase (NMT) inhibitors. Also provided herein are methods of treating, ameliorating, and/or preventing malaria with compounds of Formula (I), including treating dormant forms of malaria known as hypnozoites.

### CROSS-REFERENCE TO RELATED APPLICATION

This application claims priority to U.S. Provisional Patent Application No. 63/477,448 entitled “N-MYRISTOYLTRANSFERASE INHIBITORS AND METHODS OF USE.” filed Dec. 28, 2022, the disclosure of which is incorporated herein by reference in its entirety.

### STATEMENT REGARDING FEDERALLY SPONSORED RESEARCH

This invention was made with government support under AI155536 and AI151344 awarded by the National Institutes of Health. The government has certain rights in the invention.

### INCORPORATION-BY-REFERENCE OF MATERIAL SUBMITTED AS A TEXT FILE

This invention contains one or more sequences in a computer readable format in an accompanying text file titled “371255-7004WO1_Sequence_Listing_ST26,” which is 2 KB in size and was created on Dec. 22, 2023, the contents of which are incorporated herein by reference in their entirety.

### BACKGROUND

Malaria is a mosquito-borne disease caused by a parasitic infection. In 2020, there were approximately 241 million patients affected with malaria worldwide. Of those, 627,000 people died. There are five different species of parasites that cause malaria in humans, among which Plasmodium falciparum; and Plasmodium vivax, pose the greatest threat. In sub-Saharan Africa, most cases of malaria and mortality are caused by P. falciparum, while P. vivax is globally ubiquitous and causes persistent infections. P. vivax is highly prevalent in the Americas and Southeast Asia, accounting for 75% and 47% of all malaria cases in these regions, respectively.

Antimalarials currently available in the clinic include artemisinin-based combination therapy (ACTs), chloroquine phosphate, sulfadoxine/pyrimethamine, mefloquine, primaquine phosphate, halofantrine, and quinine. However, the emergence and persistence of resistance to almost all these drugs, and the threat of the continued robust spread of drug tolerance and resistance, necessitate the urgent development of new antimalarials. Additionally, even without resistance, many of the molecules do not successfully target the P. vivax dormant form, called hypnozoites, which are the source of all relapsing infections.

The present disclosure solves this unmet need by providing compounds that inhibit the life-cycle of malaria-causing parasites.

### BRIEF SUMMARY OF THE INVENTION

In one aspect, a compound Formula (I), or a pharmaceutically acceptable salt or solvate thereof:

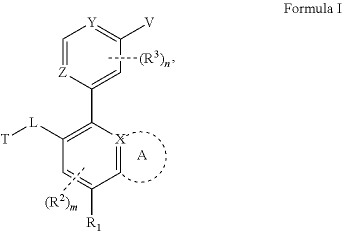

is provided. In one aspect, a pharmaceutical composition that includes at least one compound of Formula (I) and at least one pharmaceutically acceptable carrier or excipient is also provided.

In certain embodiments, in the compound of Formula (I):
A is an optionally fused 6-membered aryl or heteroaryl ring, which is optionally substituted by at least one substituent selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
X is C, CH, or N, provided that if A is present then X is C;
Z is CH or N;
Y is CH or N;
R¹ is selected from the group consisting of hydrogen, F, Cl, and Br;
L is O, NR, or ═N—*, wherein the bond with * is attached to the aryl ring;
T is —C₁₋₆ alkyl-C₅₋₆ heteroaryl, wherein the C₅₋₆ heteroaryl is optionally substituted with at least one selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
V is —C₀₋₆ alkyl-C₅₋₆ heterocyclyl, —O—C₀₋₆ alkyl-C₅₋₆ heterocyclyl, —C₀₋₆ alkyl-NR₂, or —O—C₂₋₆ alkyl-NR₂, wherein the C₅₋₆ heterocyclyl is optionally substituted with at least one selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
R is independently at each occurrence selected from the group consisting of hydrogen or optionally substituted C₁₋₁₂ alkyl;
R² is independently at each occurrence selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
R³ is independently at each occurrence selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
m is 0, 1, 2, or 3; and
n is 0, 1, 2, 3, or 4.

Also provided are methods of treating, ameliorating, and/or preventing malaria in a subject in need thereof using the compounds of Formula (I). In various aspects, the subject is infected by Plasmodium falciparum or Plasmodium vivax.

### BRIEF DESCRIPTION OF THE FIGURES

The drawings illustrate generally, by way of example, but not by way of limitation, various embodiments of the present application.

FIGS. 1A-1D show a hybridization approach for designing certain compounds of the disclosure. (FIG. TA) Chemical structures of IMP-1002 and DDD85646. (FIG. 1B) IMP-1002 and DDD85646 bound to PvNMT superimposed (PDB codes 6MBI and 2YND, respectively). (FIG. 1C) Design of target molecules: cores A and B (red), tail group (blue), and head group (brown). (FIG. 1D) Example of a designed hybrid compound (12e) docked into the crystal structure of PvNMT (PDB code 6MB1). Surface representations in front of the binding site are removed for clarity. Interactions involving the atoms of IMP-1002. DDD85646, and compound 12e are drawn using dashed lines, and water molecules are represented by red spheres.

FIGS. 2A-2D show a structural basis of PvNMT inhibition by the hybrid compound 12b, according to various embodiments. (FIG. 2A) Overall architecture. The structure of PvNMT displays the NMT fold containing an NMT N-terminal domain (tan, residues 1-204, truncated at residue 27) and an NMT C-terminal domain (cyan, residues 205-410). 12b binds in the substrate peptide binding cleft at the interface of the two domains, with the Ab loop (dark red, residues 95-102) in the closed conformation. (FIG. 2B) 12b contains the head group of DDD85646, tail group of IMP-1002, and a similar core topology as IMP-1002. (FIG. 2C) PvNMT-bound 12b and IMP-1002 superimpose closely and stabilize the S state of Tyr211. (FIG. 2D) The piperazine group of 12b occupies a binding site location similar to that of DDD85646 but interacts differently with nearby polar sites of PvNMT via N4. In FIGS. 2C and 2D, selected residues are shown in stick representation, with aligned residues from IMP-1002- and DDD85646-bound PvNMT colored black. Hydrogen bonds involving the atoms of compound 12b are drawn using dashed lines, and water molecules are represented by red spheres.

FIGS. 3A-3D illustrate that NMT inhibitors show minimal toxicity in human hepatoma cells and reduce Plasmodium falciparum: blood-stage development. (FIG. 3A) Percentage of live HepG2 cells after 48-hour treatment with each NMT inhibitor compared with that of controls treated with diluent. The staurosporine-positive control is indicated in red. (FIGS. 3B-3D) Percentage change in P. falciparum blood-stage development as measured by parasite DNA level fluorescence assay after 72-hour treatment with NMT inhibitors from each of the three prioritized compound families. Data are normalized to untreated controls set at 100%.

FIGS. 4A-4D show that NMT inhibitors reduce Plasmodium vivax schizont infection in vitro. (FIG. 4A) Representative image of schizont parasite at 8 dpi. Scale bar: 10 μm. (FIG. 4B) Effect of NMT inhibitor compounds on schizont infection levels at 8 dpi in three independent parasite isolates. All compounds were used at 20 μM, with DMSO as a control. Dose-response curves for each compound, grouped by family, for (FIG. 4C) isolate 2 and (FIG. 4D) isolate 3. Error bars represent the standard deviation of two to three technical replicates.

FIGS. 5A-5E show that NMT inhibitors reduce P. vivax hypnozoite forms in vitro. (FIG. 5A) Representative image of hypnozoite form 8 dpi. Scale bar: 10 m. (FIG. 5B) Schizont and hypnozoite infection levels for each isolate. (FIG. 5C) Effect of NMT inhibitor compounds on schizont infection levels 8 dpi for 2 independent parasite isolates. All compounds were administered at 20 μM with DMSO as a control. Error bars represent the standard deviation of 2-3 technical replicates. (FIG. 5D) Dose-response curves for each compound, grouped by family, for isolate 2. (FIG. 5E) Correlations between IC₅₀ against P. falciparum blood-stage (Pf BS). P. vivax schizont (Pv schiz), and P. vivax hypnozoite (Pv hyp) infection plotted against each other for each NMTi.

FIGS. 6A-6B show conformational changes in pocket 8. Crystal structures of PvNMT-IMP-1002 (orange. PDB entry 6MB1), PvNMT-DDD85646 (dark magenta, PDB entry 2YND), and peptide-bound HsNMT1 (pink. PDB entry 6QRM) were superimposed onto that of PvNMT-12b using Chimera. (FIG. 6A) 12b induces conformational changes in Phe226 and His213 that narrow pocket 8 of PvNMT to distances incompatible with peptide binding. (FIG. 6B) The configuration of Phe226 and His213 is associated with a conformational change in residues 217-247, boxed in Ca trace (left) and enlarged (right).

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃). —CH═C(CH₃)₂, —C(CH₃)=CH₂, —C(CH₃)═CH(CH₃), —C(CH₂CH₃)=CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms. 2 to about 20 carbon atoms, or from 2 to 12 carbons or, in some embodiments, from 2 to 8 carbon atoms. Examples include, but are not limited to —C═CH, —C═C(CH₃), —C═C(CH₂CH₃), —CH₂C═CH, —CH₂C═C(CH₃), and —CH₂C═C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines: R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcyclohexyl)-2-methylethyl, and 5,6-epoxyhexyl.

The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R. N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. A heterocyclyl ring designated C_{x-y} can be any ring containing ‘x’ members up to ‘y’ members, including all intermediate integers between ‘x’ and ‘y’ and that contains one or more heteroatoms, as defined herein. In a ring designated C_{x-y}, all non-heteroatom members are carbon. Heterocyclyl rings designated C_{x-y} can also be polycyclic ring systems, such as bicyclic or tricyclic ring systems. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl(2-thienyl, 3-thienyl), furyl(2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl(2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl(2-oxazolyl, 4-oxazolyl, 5-oxazolyl), thiazolyl(2-thiazolyl, 4-thiazolyl, 5-thiazolyl), pyridyl(2-pyridyl, 3-pyridyl, 4-pyridyl), pyrimidinyl(2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl(2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl(2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl(2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl(2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz [b,f] azepine (5H-dibenz [b,f] azepin-1-yl, 5H-dibenz [b,f] azepine-2-yl, 5H-dibenz [b,f] azepine-3-yl, 5H-dibenz [b,f] azepine-4-yl, 5H-dibenz [b,f] azepine-5-yl), 10,11-dihydro-5H-dibenz [b,f] azepine (10,11-dihydro-5H-dibenz [b,f] azepine-1-yl, 10,11-dihydro-5H-dibenz [b,f] azepine-2-yl, 10,11-dihydro-5H-dibenz [b,f] azepine-3-yl, 10,11-dihydro-5H-dibenz [b,f] azepine-4-yl, 10,11-dihydro-5H-dibenz [b,f] azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

As used herein, the term “C₆₋₁₀-5-6 membered heterobiaryl” means a C₆₋₁₀ aryl moiety covalently bonded through a single bond to a 5- or 6-membered heteroaryl moiety. The C₆₋₁₀ aryl moiety and the 5-6-membered heteroaryl moiety can be any of the suitable aryl and heteroaryl groups described herein. Non-limiting examples of a C₆₋₁₀-5-6 membered heterobiaryl include

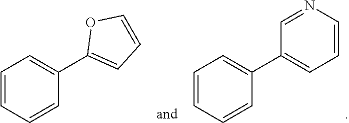 [SMILES: c1ccc(-c2ccco2)cc1 ; c1ccc(-c2cccnc2)cc1]

When the C₆₋₁₀-5-6 membered heterobiaryl is listed as a substituent (e.g., as an “R” group), the C₆₋₁₀-5-6 membered heterobiaryl is bonded to the rest of the molecule through the C₆₋₁₀ moiety.

As used herein, the term “5-6 membered-C₆₋₁₀ heterobiaryl” is the same as a C₆₋₁₀-5-6 membered heterobiaryl, except that when the 5-6 membered-C₆₋₁₀ heterobiaryl is listed as a substituent (e.g., as an “R” group), the 5-6 membered-C₆₋₁₀ heterobiaryl is bonded to the rest of the molecule through the 5-6-membered heteroaryl moiety.

As used herein, the term “C₆₋₁₀-C₆₋₁₀ biaryl” means a C₆₋₁₀ aryl moiety covalently bonded through a single bond to another C₆₋₁₀ aryl moiety. The C₆₋₁₀ aryl moiety can be any of the suitable aryl groups described herein. Non-limiting example of a C₆₋₁₀-C₆₋₁₀ biaryl include biphenyl and binaphthyl.

The term “heterocycloalkyl” as used herein refers to a cycloalkyl group as defined herein in which one or more carbon atoms in the ring are replaced by a heteroatom such as O, N, S, P, and the like, each of which may be substituted as described herein if an open valence is present, and each may be in any suitable stable oxidation state.

The term “heteroalkyl” as used herein refers to a alkyl group as defined herein in which one or more carbon atoms in the ring are replaced by a heteroatom such as O, N, S, P, and the like, each of which may be substituted as described herein if an open valence is present, and each may be in any suitable stable oxidation state.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. The term heterocyclyl includes rings where a CH₂ group in the ring is replaced by one or more C═O groups, such as found in cyclic ketones, lactones, and lactams. Examples of heterocyclyl groups containing a C—O group include, but are not limited to, β-propiolactam, γ-butyrolactam, δ-valerolactam, and ε-caprolactam, as well as the corresponding lactones. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo (carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SOSR, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “potency” refers to the dose needed to produce half the maximal response (ED₅₀).

The term “room temperature” as used herein refers to a temperature of about 15° C. to 28° C.

The term “standard temperature and pressure” as used herein refers to 20° C., and 101 kPa.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The substitution can be direct substitution, whereby the hydrogen atom is replaced by a functional group or substituent, or an indirect substitution, whereby an intervening linker group replaces the hydrogen atom, and the substituent or functional group is bonded to the intervening linker group. A non-limiting example of direct substitution is: RR—H→RR—Cl, wherein RR is an organic moiety/fragment/molecule. A non-limiting example of indirect substitution is: RR—H→RR-(LL)_{zz}-Cl, wherein RR is an organic moiety/fragment/molecule, LL is an intervening linker group, and ‘zz’ is an integer from 0 to 100 inclusive. When zz is 0, LL is absent, and direct substitution results. The intervening linker group LL is at each occurrence independently selected from the group consisting of —H, —O—, —OR, —S—, —S(═O)—, —S(═O)₂—, —SR, —N(R)—, —NR₂, —CR═, —C═, —CH₂—, —CHR—, —CR₂—, —CH₃, —C(═O)—, —C(═NR)—, and combinations thereof. (LL)_{zz} can be linear, branched, cyclic, acyclic, and combinations thereof.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Preparation of Compounds

Compounds of Formula (I) or otherwise described herein can be prepared by the general schemes described herein, using the synthetic method known by those skilled in the art. The following examples illustrate non-limiting embodiments of the compound(s) described herein and their preparation.

In various embodiments, a compound of Formula (I), or a pharmaceutically acceptable salt or solvate thereof is provided and has the structure:

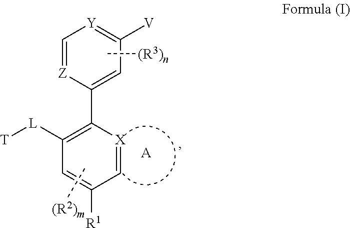

wherein
A is an optionally fused 6-membered aryl or heteroaryl ring, which is optionally substituted by at least one substituent selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
X is C, CH, or N, provided that if A is present then X is C;
Z is CH or N;
Y is CH or N;
R¹ is selected from the group consisting of hydrogen, F, Cl, and Br;
L is O, NR, or ═N—*;
T is —C₁₋₆ alkyl-C₅₋₆ heteroaryl, wherein the C₅₋₆ heteroaryl is optionally substituted with at least one selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
V is —C₀₋₆ alkyl-C₅₋₆ heterocyclyl, —O—C₀₋₆ alkyl-C₅₋₆ heterocyclyl, —C₀₋₆ alkyl-NR₂, or —O—C₂₋₆ alkyl-NR₂, wherein the C₅₋₆ heterocyclyl is optionally substituted with at least one selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
R is independently at each occurrence selected from the group consisting of hydrogen or optionally substituted C₁₋₁₂ alkyl;
R² is independently at each occurrence selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
R³ is independently at each occurrence selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
m is 0, 1, 2, or 3; and
n is 0, 1, 2, 3, or 4.

In certain embodiments, the optionally substituted C₁₋₁₂ alkyl is substituted with at least one selected from the group consisting of C₁-C₆ alkyl, halogen, cyano, —OH, C₁-C₆ alkoxy, C₁-C₆ haloalkoxy, —NH₂, —NH (C₁-C₆ alkyl), N(C₁-C₆ alkyl) (C₁-C₆ alkyl), C(O)H, and —C(O)C₁-C₆ alkyl, —C(O) OH, and —C(O)OC₁-C₆ alkyl.

In various embodiments, the compound of Formula (I) has the structure:

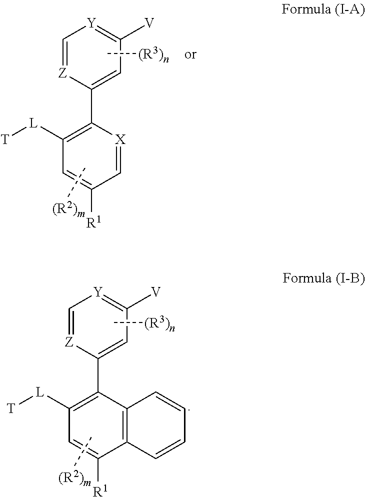

In various embodiments, L is O.

In various embodiments, L is NH.

In various embodiments, L is ═N—*, where the * indicates the bond attached to the aryl ring. When an alkyl group in T is attached to the double bond in ═N—, an imine is formed.

In various embodiments, R¹ is H or F. In various embodiments, R¹ is H. In various embodiments, R¹ is F.

In various embodiments, T is —C₁₋₆ alkyl-Cs heteroaryl. In various embodiments, T is —C₁₋₆ alkyl-C₆ heteroaryl. In various embodiments, T is —C₂ alkyl-C₅₋₆ heteroaryl. In various embodiments, T is —C₂ alkyl-Cs heteroaryl. In various embodiments, T is —C₂ alkyl-C₆ heteroaryl. The C₅ or C₆ heteroaryl in T can be attached to any open valence on the C₁₋₆ alkyl group, which can be straight or branched.

In various embodiments, T is selected from the group consisting of:

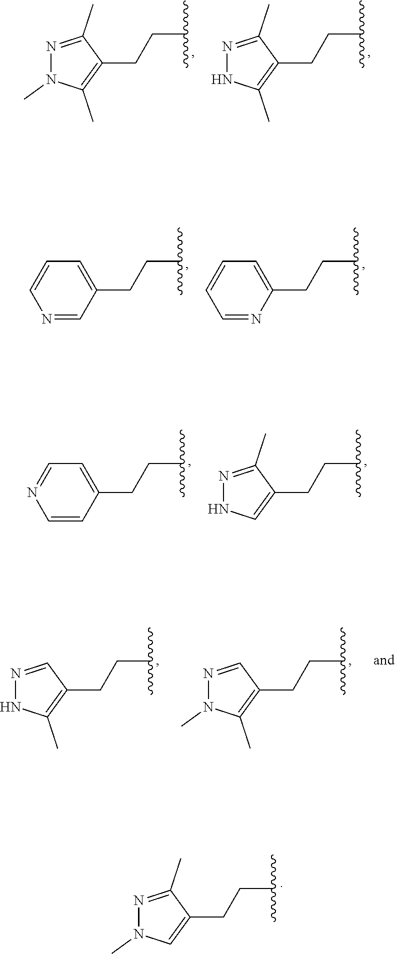

wherein a methyl (CH₃) group in any of the foregoing can optionally be replaced by RT.

In various embodiments, T is selected from the group consisting of:

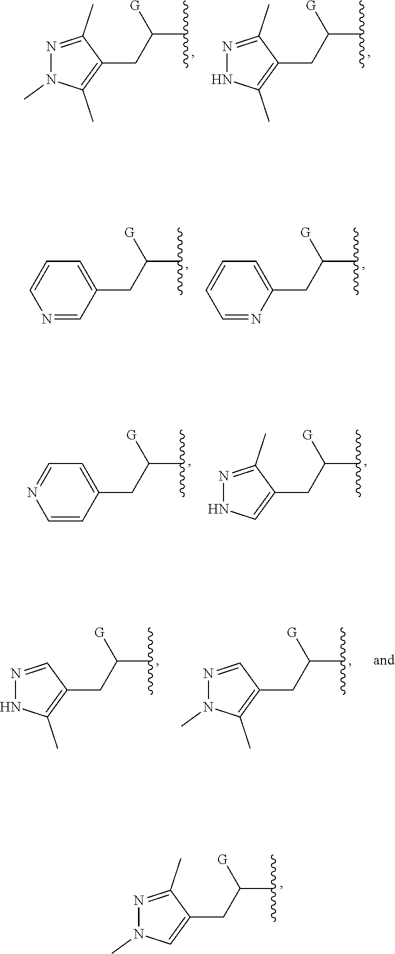

wherein a methyl (CH₃) group in any of the foregoing can optionally be replaced by R^{T}, and G is independently at each occurrence CH₃, CH₂CH₃, or CH₂CH₂OH. Contemplated herein are all enantiomers of the compounds of Formula (I) when T is a group containing G.

In certain embodiments, R^{T} is —C(═O)—C₁₋₆ heteroalkyl, —C(═O)—C₆ heterocycloalkyl, —C(═O)—C₆ aryl, —C(═O)—C₆ heteroaryl, —C(═O)—NH—C₆ aryl, or —C(═O)—NH—C₆ heteroaryl, where the C₁₋₆ heteroalkyl is optionally substituted by F, Cl, Br, OR, or ═O. In various embodiments, the C₁₋₆ heteroalkyl is a C₁₋₆ alkylamine.

In various embodiments, T is:

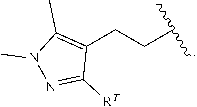

In various embodiments, R^{T} is selected from the group consisting of

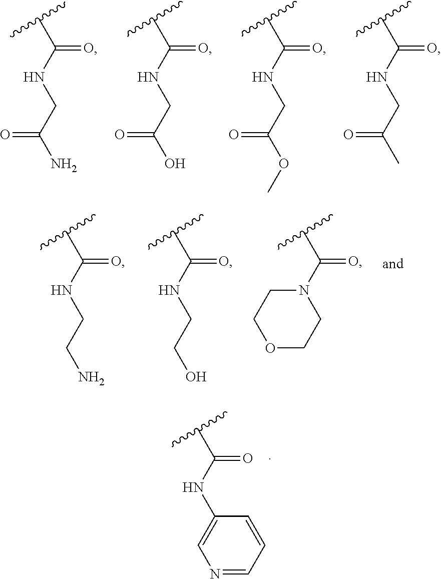

In various embodiments, m is 0. In various embodiments, n is 0. In various embodiments, V is C₅₋₆ heterocyclyl. In various embodiments, V is —C₁₋₆ alkyl-C₅₋₆ heterocyclyl or —O—C₂₋₆ alkyl-NR₂. In various embodiments, is —C₀₋₆ alkyl-Cs heterocyclyl. In various embodiments, is —C₀₋₆ alkyl-C₆ heterocyclyl. The designation Co in V means that no methylene group is present, and the C₅₋₆ heterocyclyl group is directly connected to the rest of the compound by a bond. The C₅₋₆ heterocyclyl group in V can be an heteroaromatic group or heterocyclic group with one, two, or three fewer double bonds than the corresponding aromatic group. In various embodiments, the C₅₋₆ heterocyclyl group in V is a C₅₋₆ heterocycloalkyl. In various embodiments, V is

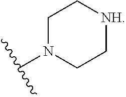 [SMILES: *N1CCNCC1]

The C₅ or C₆ heterocyclyl in V can be attached to any open valence on the C₁₋₆ alkyl group, which can be straight or branched.

In various embodiments, V is —O—C₂₋₆ alkyl-NR₂. In various embodiments, V is —O—C₂ alkyl-NR₂. In various embodiments, each R in an NR₂ group in V is methyl, ethyl, propyl, cyclopropyl, iso-propyl, butyl, sec-butyl, or t-butyl. In various embodiments, V is

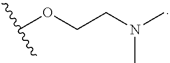 [SMILES: *OCCN(C)C]

In various embodiments, A is

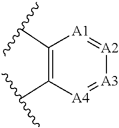

wherein
each A1, A2, A3, and A4 is independently CH or N, provided no more than two of A1, A2, A3, and A4 are N;
each CH group in A1 to A4 is optionally independently substituted by a substituent selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂.

In various embodiments, NR in A1 to A4, when present in a substituent, is NH.

The wavy lines in structure A represent connection points to the rest of the compound in the structure of Formula (I).

In various embodiments, A is

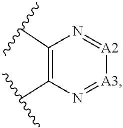

and each of A2 and A3 is a CH group optionally independently substituted as described herein.

In various embodiments, A is

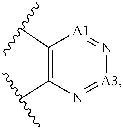

and each of A1 and A3 is a CH group optionally independently substituted as described herein.

In various embodiments, A is

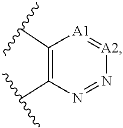

and each of A1 and A2 is a CH group optionally independently substituted as described herein.

In various embodiments, A is

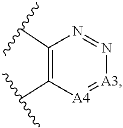

and each of A3 and A4 is a CH group optionally independently substituted as described herein.

In various embodiments, A is

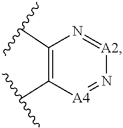

and each of A2 and A4 is a CH group optionally independently substituted as described herein.

In various embodiments, A is

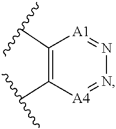

and each of A1 and A4 is a CH group optionally independently substituted as described herein.

In various embodiments, A is

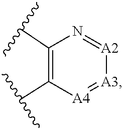

and each of A2, A3, and A4 is a CH group optionally independently substituted as described herein.

In various embodiments, A is

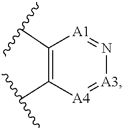

and each of A1, A3, and A4 is a CH group optionally independently substituted as described herein.

In various embodiments, A is

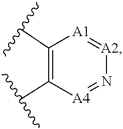

and each of A1, A2, and A4 is a CH group optionally independently substituted as described herein.

In various embodiments, A is

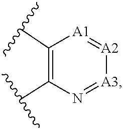

and each of A1, A2, and A3 is a CH group optionally independently substituted as described herein.

In various embodiments, the compound of Formula (I) is at least one compound selected from the group consisting of:

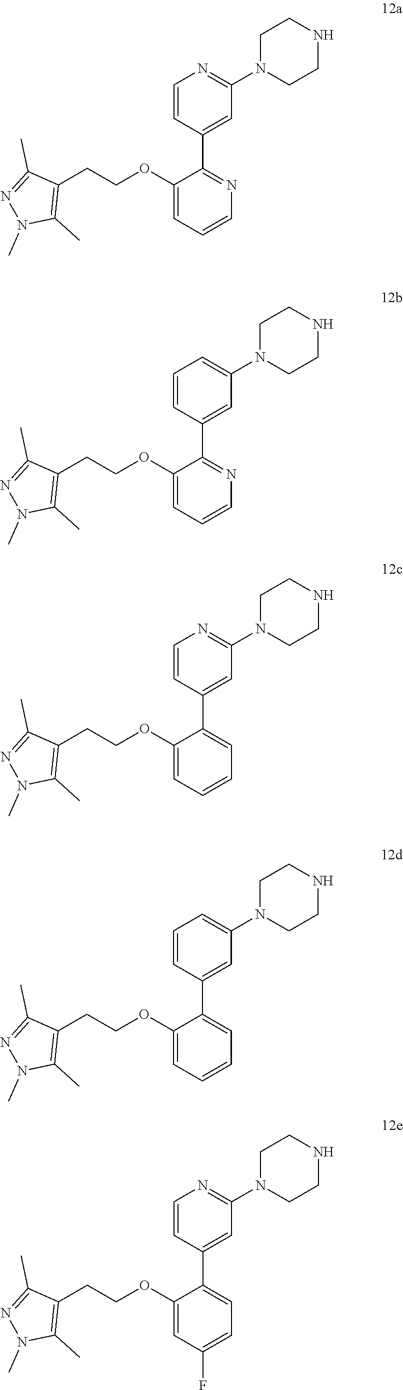 [SMILES: Cc1nn(C)c(C)c1CCOc1cccnc1-c1ccnc(N2CCNCC2)c1 ; Cc1nn(C)c(C)c1CCOc1cccnc1-c1cccc(N2CCNCC2)c1 ; Cc1nn(C)c(C)c1CCOc1ccccc1-c1ccnc(N2CCNCC2)c1 ; Cc1nn(C)c(C)c1CCOc1ccccc1-c1cccc(N2CCNCC2)c1 ; Cc1nn(C)c(C)c1CCOc1cc(F)ccc1-c1ccnc(N2CCNCC2)c1]

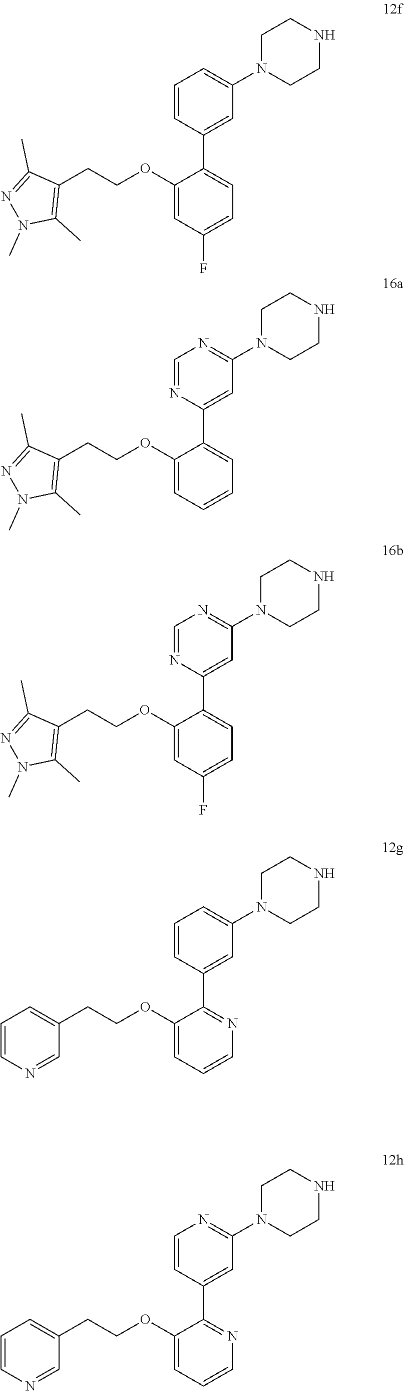 [SMILES: Cc1nn(C)c(C)c1CCOc1cc(F)ccc1-c1cccc(N2CCNCC2)c1 ; Cc1nn(C)c(C)c1CCOc1ccccc1-c1cc(N2CCNCC2)ncn1 ; Cc1nn(C)c(C)c1CCOc1cc(F)ccc1-c1cc(N2CCNCC2)ncn1 ; c1cncc(CCOc2cccnc2-c2cccc(N3CCNCC3)c2)c1 ; c1cncc(CCOc2cccnc2-c2ccnc(N3CCNCC3)c2)c1]

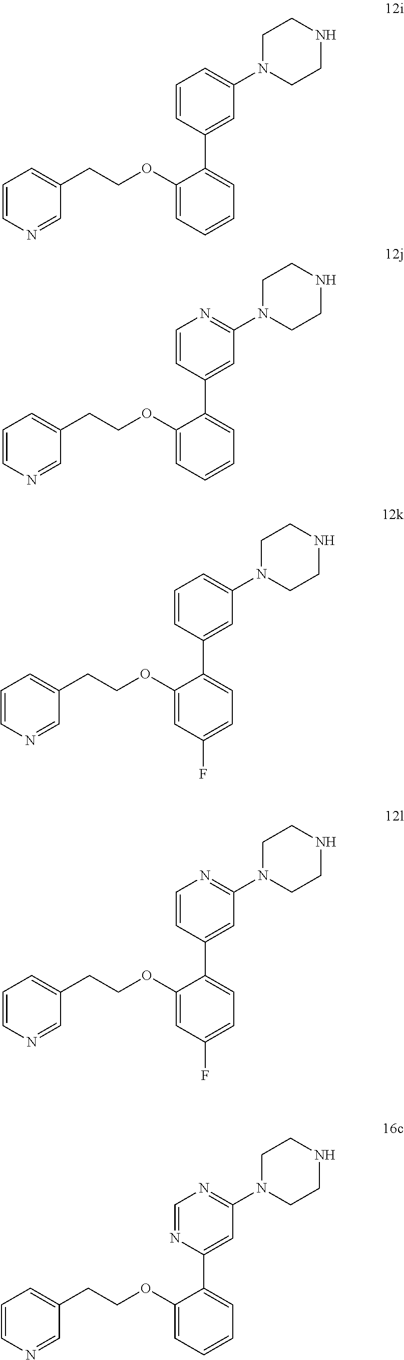 [SMILES: c1cncc(CCOc2ccccc2-c2cccc(N3CCNCC3)c2)c1 ; c1cncc(CCOc2ccccc2-c2ccnc(N3CCNCC3)c2)c1 ; Fc1ccc(-c2cccc(N3CCNCC3)c2)c(OCCc2cccnc2)c1 ; Fc1ccc(-c2ccnc(N3CCNCC3)c2)c(OCCc2cccnc2)c1 ; c1cncc(CCOc2ccccc2-c2cc(N3CCNCC3)ncn2)c1]

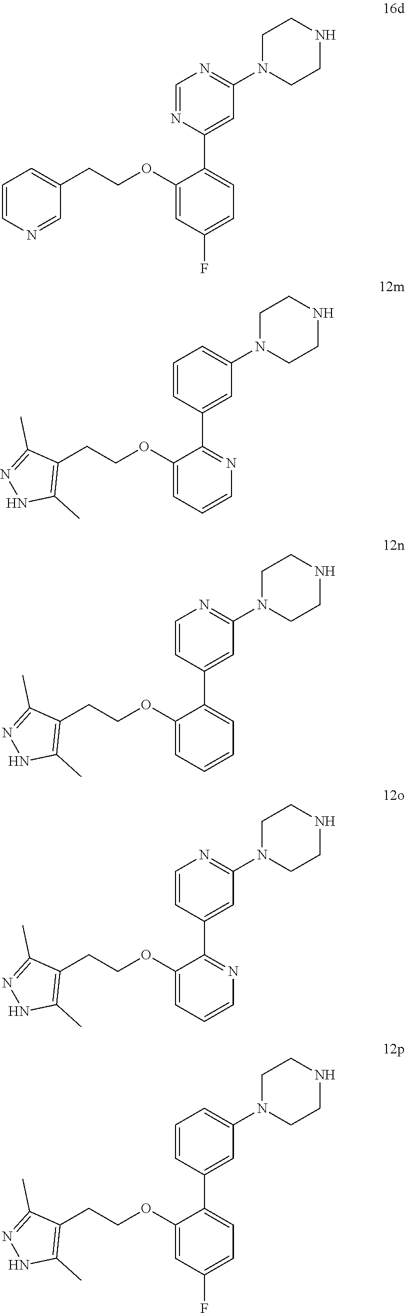 [SMILES: Fc1ccc(-c2cc(N3CCNCC3)ncn2)c(OCCc2cccnc2)c1 ; Cc1n[nH]c(C)c1CCOc1cccnc1-c1cccc(N2CCNCC2)c1 ; Cc1n[nH]c(C)c1CCOc1ccccc1-c1ccnc(N2CCNCC2)c1 ; Cc1n[nH]c(C)c1CCOc1cccnc1-c1ccnc(N2CCNCC2)c1 ; Cc1n[nH]c(C)c1CCOc1cc(F)ccc1-c1cccc(N2CCNCC2)c1]

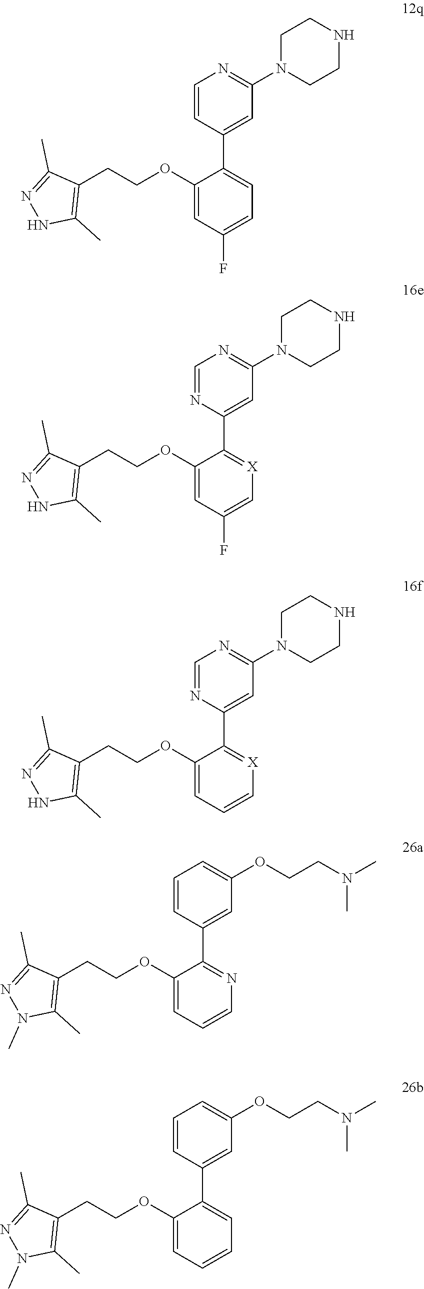

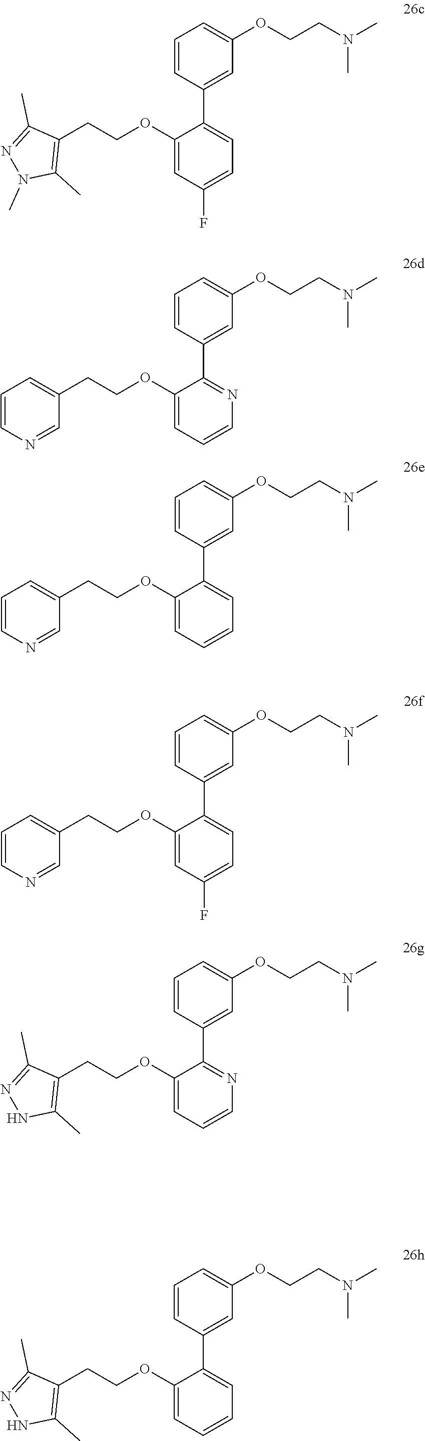

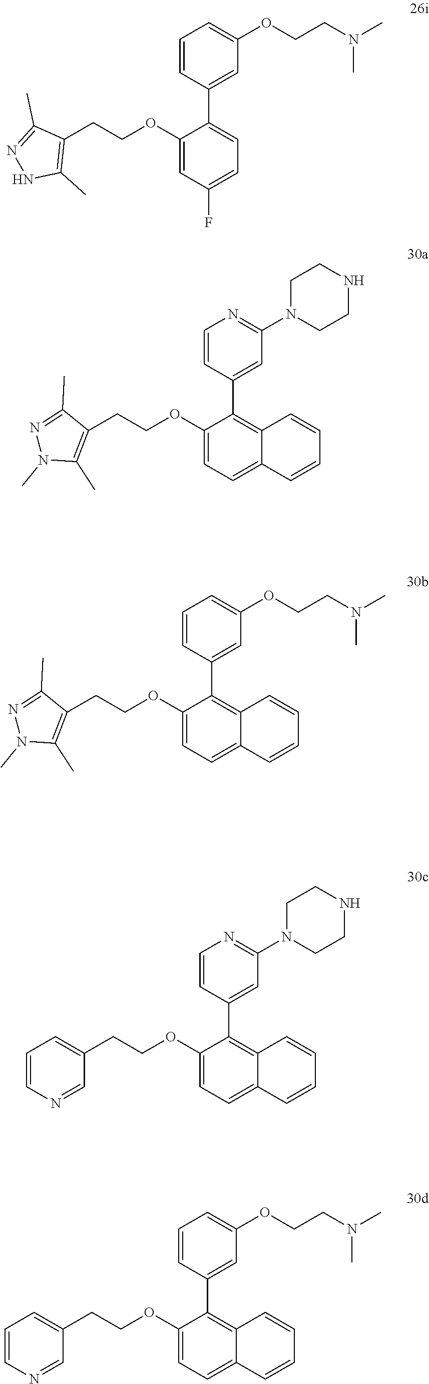

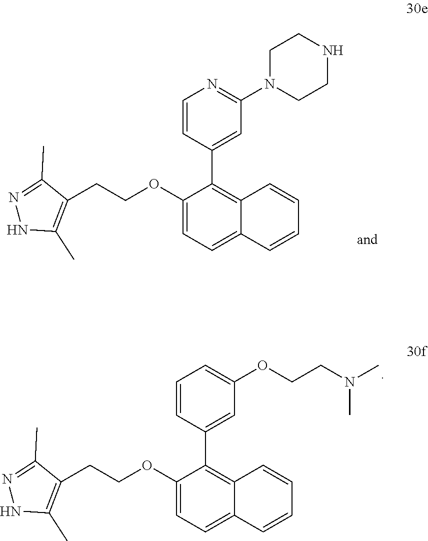 [SMILES: Cc1n[nH]c(C)c1CCOc1ccc2ccccc2c1-c1ccnc(N2CCNCC2)c1 ; Cc1n[nH]c(C)c1CCOc1ccc2ccccc2c1-c1cccc(OCCN(C)C)c1]

Targeting Plasmodium parasites towards the goal of eradication is best applied after careful consideration of the parasitic lifecycle. Transmission from the Anopheles mosquito host to the human host, occurs when parasites are deposited in the dermis. The parasites then travel through the skin, and ultimately enters a blood vessel. The blood stream then carries the parasites to the liver, where they cross the sinusoidal endothelium and invades a single hepatocyte. For parasites of most Plasmodium species, liver residence takes 2-10 days, at which time the parasites rapidly divide within the host hepatocyte. After liver replication, called schizogony, the parasites exit the liver, invade erythrocytes, and initiate the asexual replication cycle. This cycle is associated with all the morbidity and mortality associated with malaria disease. When these forms differentiate into sexual gametocytes forms, productive human-to-mosquito transmission occurs.

The life-cycle of P. vivax and P. ovale is slightly altered because of the existence of liver-resistant dormant forms called hypnozoites. Hypnozoites represent a major hurdle for malaria eradication efforts. Hypnozoites remain in the liver for weeks, months, or even years and later reactivate, leading to relapse and symptomatic blood-stage infection. Between 20% and 100% of P. vivax transmission events result from hypnozoite relapse, depending on the location and intensity of transmission. The current pharmacological options for the elimination of hypnozoites are limited to primaquine and tafenoquine, and the use of these drugs is hampered by their severe toxicity in individuals with glucose-6-phosphate dehydrogenase (G6PD) deficiency. Targeting regulatory proteins within the parasites that facilitate multiple stages of the complex life cycle might be the most robust approach to eliminating malaria.

Multiple antimalarials and combination therapies that can be used as single-dose regimens are being developed pre-clinically and clinically. Ambitious efforts to eliminate malaria, such as those by the Bill and Melinda Gates Foundation and the Medicines for Malaria Venture (MMV), include approaches that could target multiple stages of the parasitic life cycle. Yet, the goal of malaria eradication is far from being achieved, and several knowledge gaps remain in the current pharmacopeia of antimalarial compounds. Antimalarials that are part of several classes are needed to support a robust malaria eradication campaign. Specifically, candidate drugs targeting the asexual blood stage (target candidate profile [TCP]-1), anti-relapse/hypnozoites (TCP-3), liver schizonts (TCP-4), and transmission-blocking (TCP-5 and TCP-6) are essential for reducing malaria cases worldwide. Accordingly, compounds that can achieve multiple of these objectives would be of particular interest.

N-Myristoyltransferase (NMT) is a ubiquitous enzyme in eukaryotes that catalyzes the transfer of myristate from myristoyl-coenzyme A (myrCoA) to the N-terminal glycine residue of a nascent polypeptide at the ribosome. The myristoylation of a protein generally aids in membrane localization and stability by increasing the protein's hydrophobicity. Highly active Plasmodium NMT inhibitors (NMTis) were previously developed with moderate selectivity over the human enzyme capable of killing the parasite; some have been demonstrated NMTis inhibitors to eliminate Plasmodium liver stage and blood-stage parasites. IMP-1002 has an IC₅₀ value of 3 nM and up to fourfold selectivity over HsNMT1/2 with an EC₅₀ value of 10 nM in a P. falciparum growth assay. In addition, DDD85646, a potent inhibitor of P/NMT, has an IC₅₀ value of 40 nM and <1 selectivity over HsNMT1/2 and an EC₅₀ value of 69 nM in a P. falciparum growth assay. Pharmacological inhibition of NMT prevents the completion of blood-stage development of P. falciparum partly owing to the inhibition of the inner membrane complex formation. Thus, selective targeting of NMT could aid in the development of a drug that targets multiple life cycle stages.

Although multiple inhibitors of Plasmodium NMT have been identified, the selectivity against human NMTs remains a major challenge as Plasmodium and human NMTs exhibit a high degree of homology at the active site of the enzyme, where all known inhibitors bind. This challenge has been cited as a reason to deprioritize the targeting of parasitic NMT sites in antimalarial efforts. This study reports in part the development of Plasmodium-specific NMT inhibitors (NMTis) with strong selectivity (~270×) towards P. vivax over their human counterpart. Furthermore, this study provides a structural basis for such high selectivity through crystal structure elucidation of PvNMT bound to one of the most selective inhibitors. Selected PvNMTis were evaluated in schizont and hypnozoite infections of liver-stage P. vivax parasites and blood-stage P. falciparum parasites.

### Design and Synthesis of Selective PvNMT Inhibitors

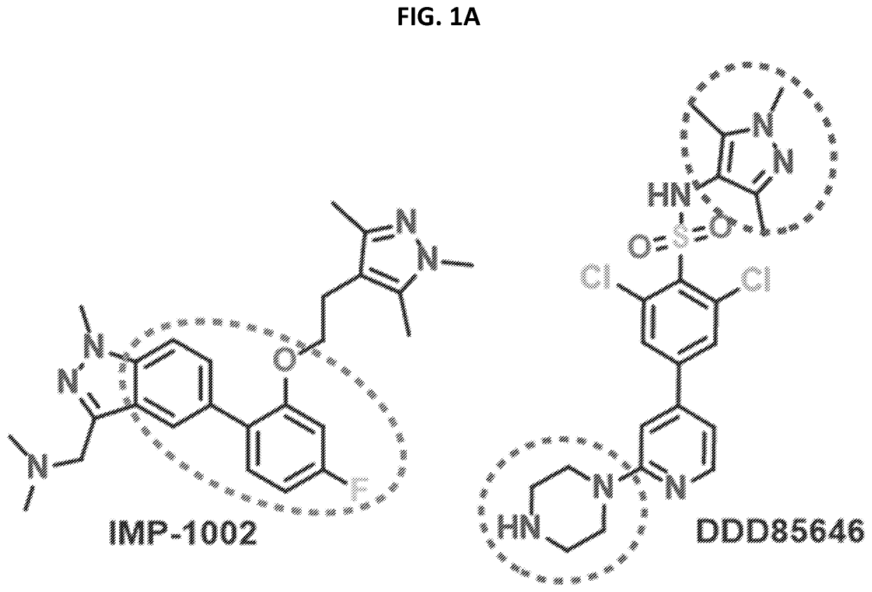

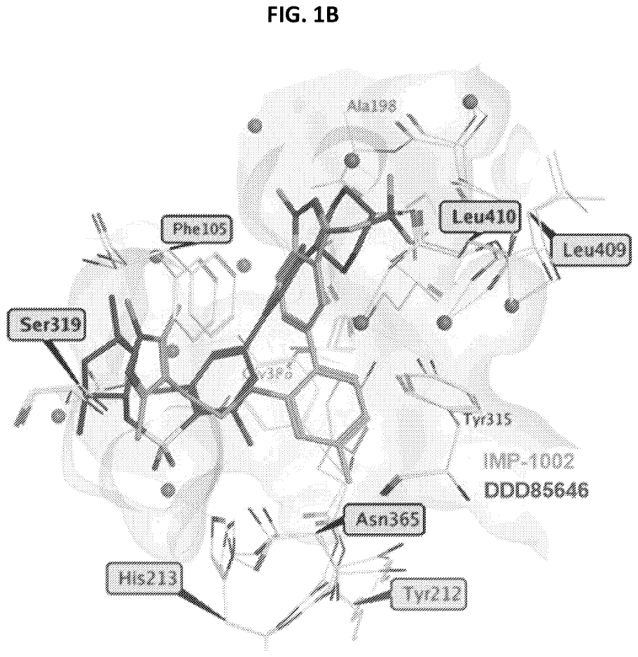

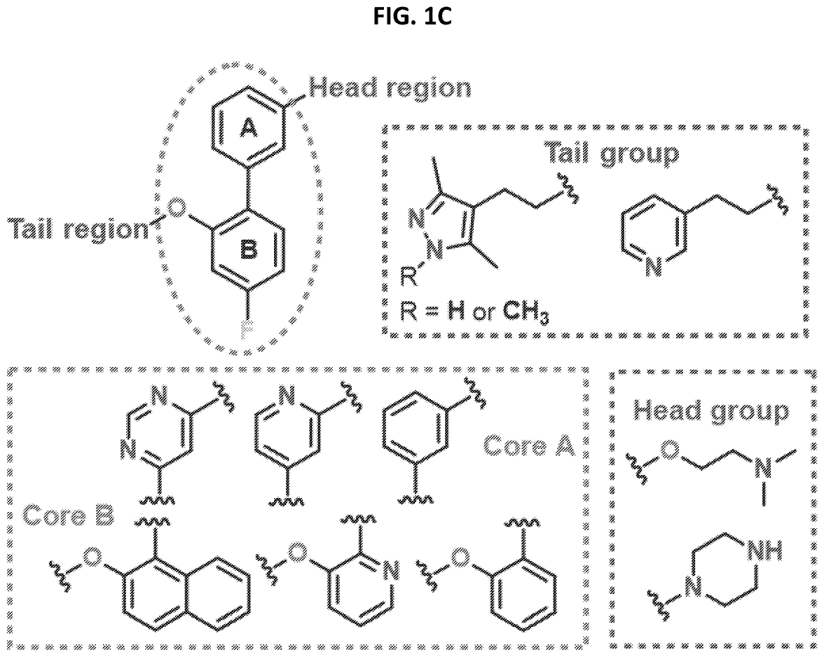

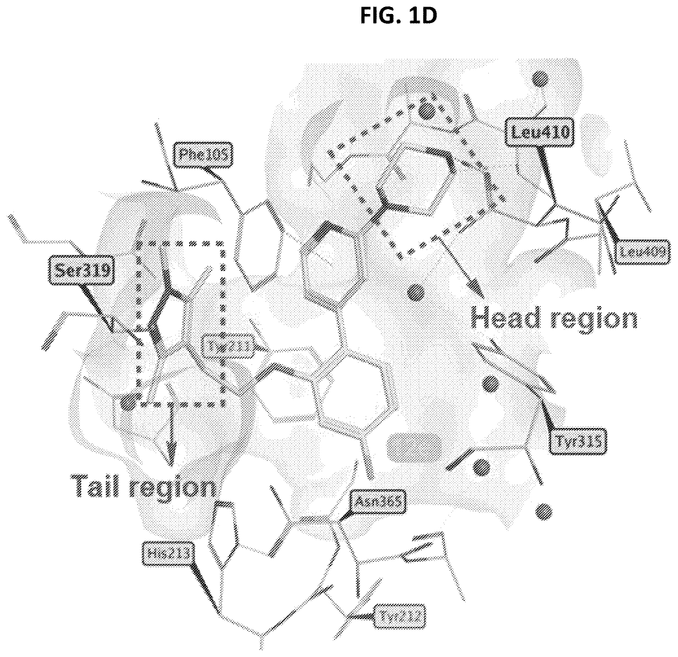

PvNMT inhibitors DDD85646 and IMP-1002 (FIG. 1A) were used as the starting point for a structure-based approach to develop more potent and selective PvNMTis. Both NMTis occupied different parts of the P. vivax NMT binding pocket, with excellent affinity and potency (FIG. 1B). Based on the crystal structures of IMP-1002 and DDD85646 bound to PvNMT (FIG. 1B), inhibitors were designed using a part of IMP-1002 (FIG. 1A, red circle) as a scaffold and fragments of DDD85646 (FIG. 1A, blue and brown circles) to build hybrid compounds. The molecular hybridization strategy was based on a “headgroup” region linked to core A and a “tail” region linked to core B (FIG. 1C), whose combination formed the biaryl scaffold.

The optimization efforts focused on altering these regions, and different aromatic rings were used to modify the biaryl scaffold (FIG. 1C, cores A and B). In the tail region (FIG. 1C, highlighted in blue), the substituents were designed to interact with Ser319. In the head region (FIG. 1C, highlighted in brown), the substituents were selected to interact ionically with the carboxylate of the C-terminal residue (Leu410), which plays an essential role in myristate transfer and is crucial in the inhibitor potency against Plasmodium NMT. FIG. 1D shows a particular hybrid compound (i.e., 12e) that was designed and synthesized to dock into the crystal structure of PvNMT with the target hydrogen bonding and ionic interactions formed with Ser319 and Leu410.

TABLE A
Structures of tail groups 1, 2, and 3 used in Table 1 and Table 2.

| Tail 1 | Tail 2 | Tail 3 |
|---|---|---|
| 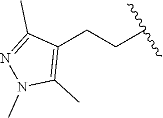 [SMILES: *CCc1c(C)nn(C)c1C] | 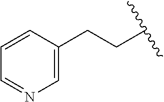 [SMILES: *CCc1cccnc1] | 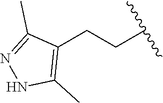 [SMILES: *CCc1c(C)n[nH]c1C] |

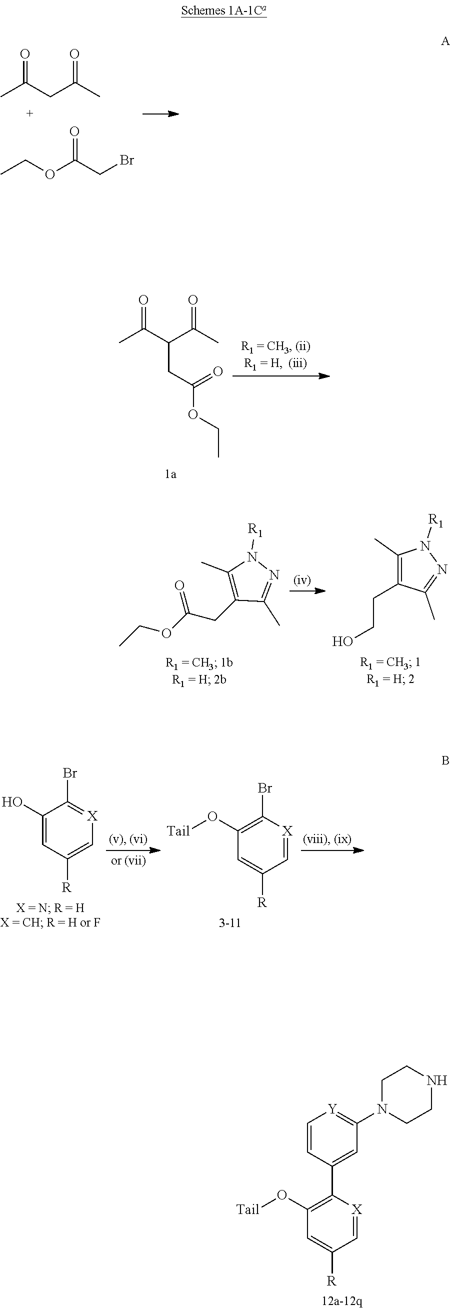

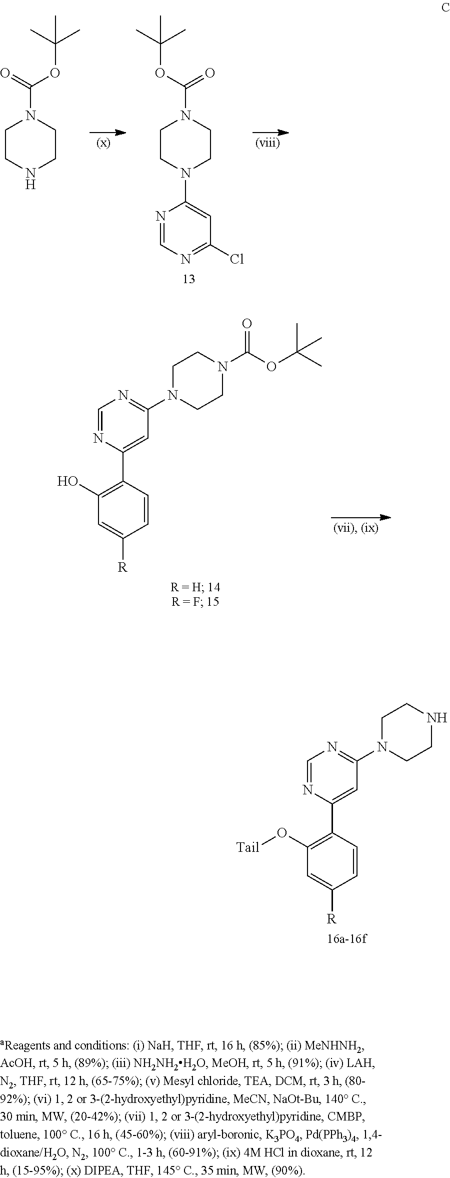

### Piperazine-Hybrid Compounds Show High Affinity and >145-Fold Selectivity

Table 1 summarizes the synthetic route used to prepare the hybrid compounds (12a-q and 16a-f). These structural fragments were prepared using a three-step synthesis strategy (Scheme 1A), with a condensation reaction as a key step. The synthesis of hybrid compounds 12a-q began with the formation of different blocks of aryl ethers (3-11) through a sulfonyl transfer or the Mitsunobu reaction (Scheme 1B). The intermediate blocks (3-11) were obtained from commercially available aryl-hydroxyl or pyridine-hydroxyl with pyrazoles 1 and 2 or with commercially available 3-(2-hydroxyethyl)pyridine. From these aryl-ethers blocks, hybrid synthesis was accomplished in two steps (Scheme 1B). A Suzuki cross-coupling reaction was used to couple cores A and B together (Scheme 1C). This was followed by Boc-deprotection using 4 M HCl in dioxane to furnish compounds 12a-q in sufficient overall yields (60-91%).

NMT activity was indirectly measured through the detection of free CoA by the thiol-reactive probe 7-diethylamino-3-(4′-maleimidylphenyl)-4-methylcoumarin (CPM). The enzymatic activities of the hybrid compounds 12a-q are summarized in Table 1. Five compounds in this series having either a 1,3,5-trimethylpyrazole (12a, 12b, and 12d) or pyridine (12g and 12j) tail group yielded a selectivity index (SI) of >145, computed as the ratio of IC₅₀ values for the HsNMT1 and PvNMT. Moreover, the three compounds bearing a 1,3,5-trimethylpyrazole tail group had IC₅₀ values below 100 nM. These results demonstrate that designing potent inhibitors having high selectivity is achievable using the combinatorial design strategy, despite the high degree of active site conservation of PvNMT and HsNMT1.

TABLE 1
Synthesis and biochemical activity of hybrid compounds bearing a piperazine
moiety as a head group.

| | | | | | | | PvNMT | HsNMT | |
|---|---|---|---|---|---|---|---|---|---|
| | | | | | | | IC₅₀ | IC₅₀ | |
| Compound | core | Y | Z | X | R | Tail | (nM)* | (nM)* | SI** |
| 12a 12b 12c 12d 12e 12f 16a 16b 12g 12h 12i 12j 12k 12l 16c 16d | 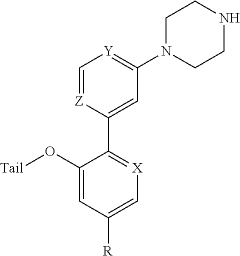 | N CH N CH N CH N N CH N CH N CH N N N | CH CH CH CH CH CH N N CH CH CH CH CH CH N N | N N CH CH CH CH CH CH N N CH CH CH CH CH CH | H H H H F F H F H H H H F F H F | 1 2 | 36.8 80.15 9.48 48.5 15.8 15.9 9390 168 104 440.6 1540 124 3120 23.4 >40000 3390 | 5400 20780 599 7440 124 699 — 27620 15900 >40000 — 27360 — 1710 — — | 146.7 259.2 63.1 153.4 7.8 43.9 — 164.4 152.8 90.9 — 220.6 — 73 — — |
| 12m | | CH | CH | N | H | 3 | 12540 | — | — |
| 12n | | N | CH | CH | H | | 604 | 39730 | 65.7 |
| 120 | | N | CH | N | H | | 3116 | — | — |
| 12p | | CH | CH | CH | F | | 368 | 23080 | 62.7 |
| 12q | | N | CH | CH | F | | 73 | 1632 | 22 |
| 16e | | N | N | CH | F | | 5070 | — | — |
| 16f | | N | N | CH | H | | 14033 | — | — |

*PvNMT and HsNMT IC₅₀ values are shown as mean values of two or more determinations .
**Enzyme selectivity is calculated as HsNMT IC₅₀/PvNMT IC₅₀ (nM).

Increasing the Polarity of the Core Scaffold with Pyrimidine Yields Mixed Results

Compounds 16a-f had a pyrimidine moiety as their core A (FIG. 1C). The introduction of a pyrimidine group increased the polarity of the hybrid compounds. To attach the piperazine head to this core, commercially available 2,4-dichloropyrimidine was reacted with Boc-protected-piperazine to generate compound 13 (Table 1C). Subsequent Suzuki coupling with the appropriate boronic acid yielded biaryl intermediates 14 and 15. The tail components were introduced via the Mitsunobu reaction, followed by Boc-deprotection using 4M HCl in dioxane, to produce the pyrimidine hybrid compounds (16a-16f) in good yields.

The introduction of a pyrimidine group in core A, motivated by the hypothesis that this group increases the polarity of the hybrid compounds, had no impact on the affinity over PvNMT (Table 1). However, compound 16b, having the same groups in the head and tail regions, was more selective over HsNMT (SI=164) than 12e or 12f.

### Dimethylaminoethanol Head Group Shows an Increased Potency

With the goal of increasing potency against PvNMT, the piperazine head group was replaced with the 2-(dimethylamino)ethanol moiety. This increased the c Log P value by approximately 0.3-0.9 log units for the different compounds presented in Table 1. The protonatable nitrogen from the dimethylamino group was expected to form a favorable ionic interaction with the carboxylate group of the C-terminal residue. Table 2A shows the synthetic route used to prepare this series of hybrid compounds (26a-i). The synthesis began with a Suzuki coupling reaction using the different blocks of aryl ethers (3-11) with the commercially available 3-hydroxyphenylboronic acid to form the biaryl scaffold (17-25). Finally, the commercially available 2-(dimethylamino)-ethanol moiety was reacted with a corresponding biaryl framework using the Mitsunobu reaction to produce the desired hybrid compounds (26a-i) in good overall yields.

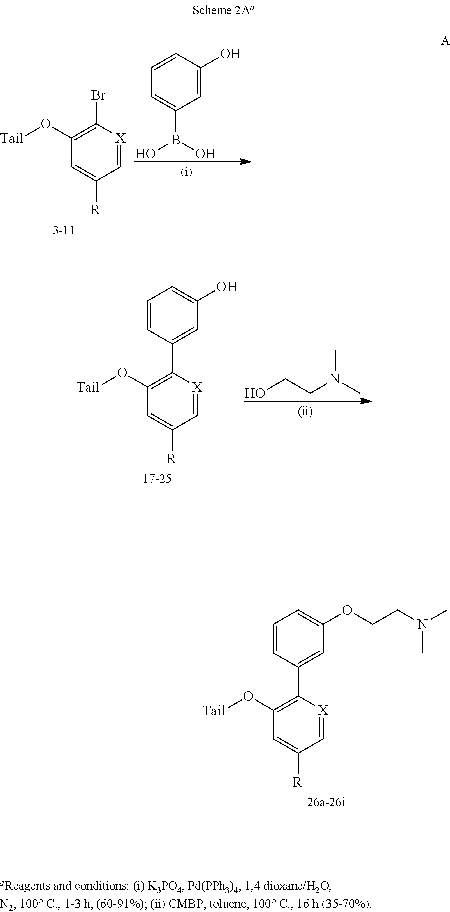

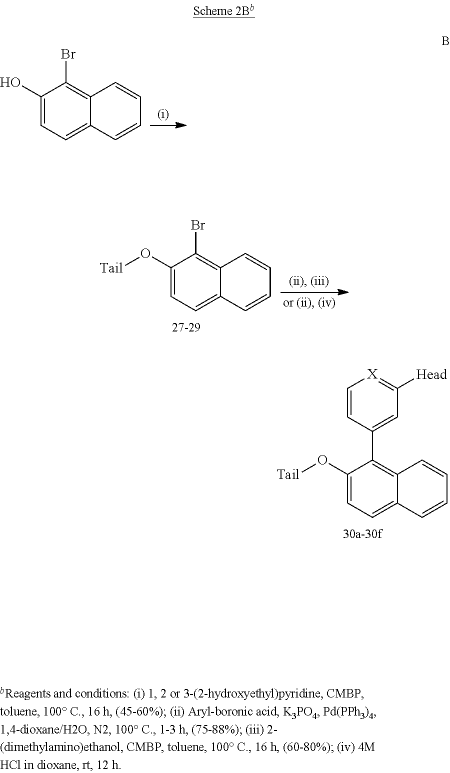

TABLE 2
Synthesis and biochemical activity of hybrid compounds bearing a dimethylamino-
ethanol moiety as a head group and hybrid compounds bearing a naphthol moiety in core B of
the biaryl scaffold.

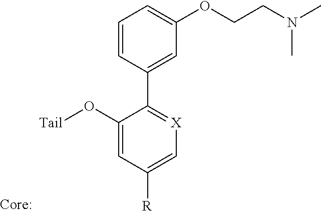

| | | | | PvNMT | HsNMT | |
|---|---|---|---|---|---|---|
| Code | X | R | Tail | IC₅₀ (nM)* | IC₅₀ (nM)* | SI** |
| 26a | N | H | 1 | 266 | >40000 | 153.8 |
| 26b | CH | H | | 83.2 | 9850 | 118.4 |
| 26c | CH | F | | 29.4 | 4790 | 162.3 |
| 26d | N | H | 2 | 14800 | — | — |
| 26e | CH | H | | 12760 | — | — |
| 26f | CH | F | | 622 | >40000 | 62.3 |
| 26g | N | H | 3 | 19400 | — | — |
| 26h | CH | H | | 1563 | — | — |
| 26i | CH | F | | 242 | 13860 | 57 |

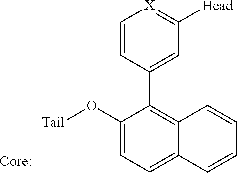

| | | | | | HsNMT | |
|---|---|---|---|---|---|---|
| | | | | PYNMT | IC₅₀ | |
| Code | X | Head | Tail | IC₅₀ (nM)* | (nM) | SI** |
| 30a | N | 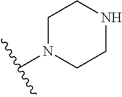 [SMILES: *N1CCNCC1] | 1 | 89 | 24010 | 269.8 |
| 30b | CH | 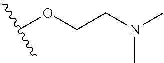 [SMILES: *OCCN(C)C] | | 19830 | — | — |
| 30c | N | 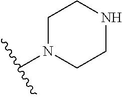 [SMILES: *N1CCNCC1] | 2 | 248 | >40000 | 161 |
| 30d | CH | 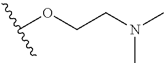 [SMILES: *OCCN(C)C] | | 7210 | — | — |
| 30e | N | 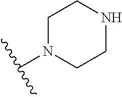 [SMILES: *N1CCNCC1] | 3 | 2880 | — | — |
| 30f | CH | 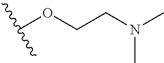 [SMILES: *OCCN(C)C] | | 25400 | — | — |

The in vitro IC₅₀ values of the hybrid compounds 26a-i are summarized in Table 2. The three compounds of this series having a 1,3,5-trimethylpyrazole group in the tail region (26a, 26b, and 26c) exhibited an SI of >115. Although the compounds with the dimethylaminoethanol group had higher c Log P values than the hybrids that had the piperazine group in the head region (12f vs 26c or 12d vs 26b), the affinity for PvNMT was similar to that of compounds with the 1,3,5-trimethylpyrazole group in the tail region with IC₅₀ values below 100 nM (see Tables 1 and 2).

Increasing the Lipophilicity of the Core Scaffold with Naphthol Improves Selectivity

Further synthetic modifications in core B were explored by introducing a polycyclic aromatic hydrocarbon such as naphthol. Through molecular docking of compound 12e (FIG. 1D), a space was observed in the pocket of the binding site, indicating that it was appropriate to introduce another ring in core B. The introduction of the naphthol group increased the lipophilicity, improving the potency and selectivity. For these hybrids, different groups of heterocycles were preserved, including the tail moiety and the piperazino-pyridine and 2-(phenoxy)-N,N-dimethyl-ethane-1-amine moiety as core A and the head moiety. To synthesize these compounds (30a-f), the same reactions presented in Tables 1A-C and 2A were employed. Table 2B displays the synthetic route used to prepare this series of hybrid compounds (30a-f) in good overall yields.

With the introduction of a naphthol group into core B, the enzymatic activity had no impact when the compounds carried dimethylaminoethanol (26b vs. 30b) in the head region. However, the affinity enhancement imparted by the piperazine group in the head region (30a vs 30b) was accompanied by an improved selectivity (Table 2). Even compound 30a had less affinity over PvNMT than compound 12c. However, compound 30a showed a better degree of selectivity over HsNMT (SI=269) than did all other hybrid compounds (Tables 1-2).

### Crystal Structure Shows Lead Compound Bound in a Selective Binding Mode.

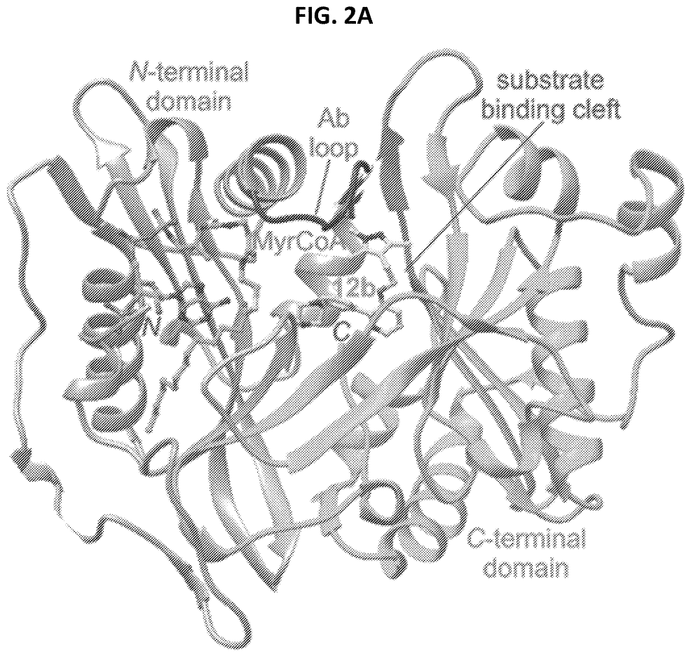

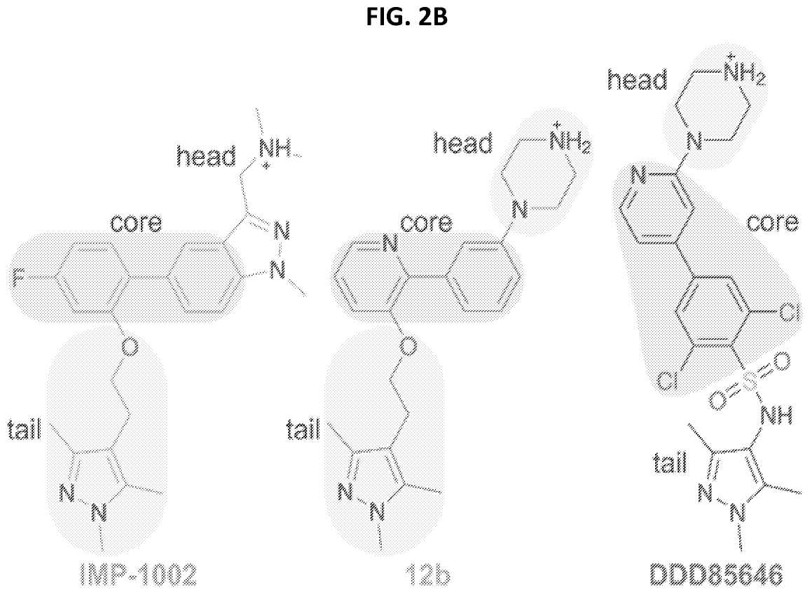

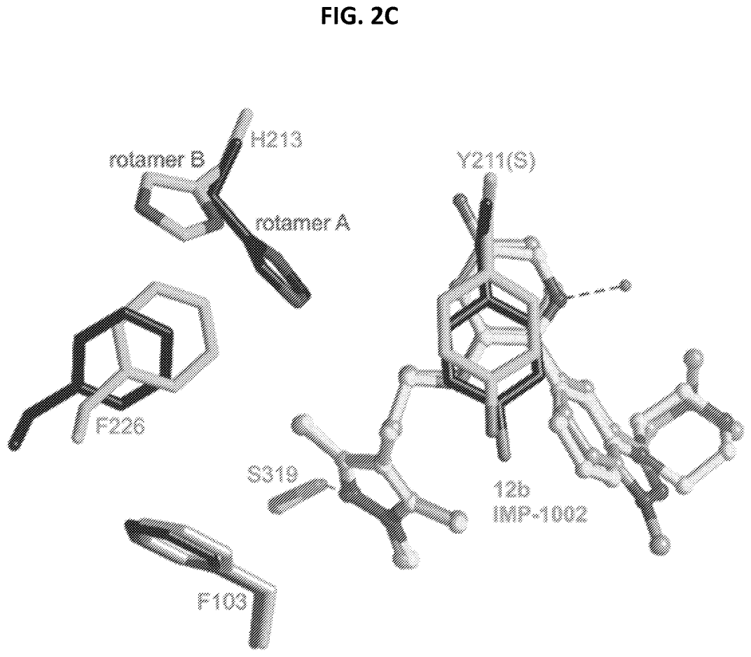

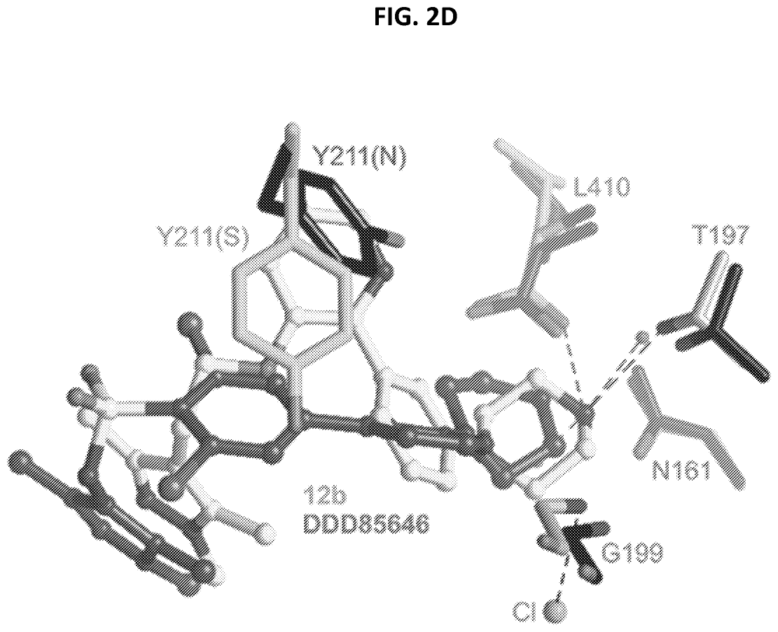

This study attempted to determine the X-ray co-crystal structures of the compounds with an SI over 200 and nano-molar affinity but was successful in obtaining only high-resolution diffraction from the co-crystals of compound 12b to 1.65 Å resolution (FIG. 2A and Table 4). Similar to other PvNMT inhibitors, 12b binds in a hydrophobic pocket formed by residues from the N- and C-terminal domains, including the Ab loop.

The 12b binding site architecture is similar to that observed for IMP-1002 bound to PvNMT [root mean square deviation (RMSD) of binding site residues of 0.9 Å; FIGS. 2B and C]. The phenyl-pyridine core of 12b stabilizes a highly rotated conformation of Tyr211 that is also observed in the IMP-1002 bound structure and which was previously proposed to represent a selective(S) state. This differs from the DDD85646-bound structure, which shows Tyr211 in a nonrotated conformation that corresponds to a nonselective state (N) (FIG. 2D). The 1,3,5-trimethylpyrazole tail group of 12b forms a hydrogen bond with Ser319, similar to IMP-1002 and as predicted from the docking calculations performed using 12e. In addition, the piperazine group of 12b binds in a similar location as that of DDD85646. However, N4 makes weaker electrostatic interactions with the C-terminal carboxylate group (separation distance of 4.0 Å). In addition, N4 is positioned more closely to Thr197, forming a hydrogen bond with the side chain hydroxyl group.

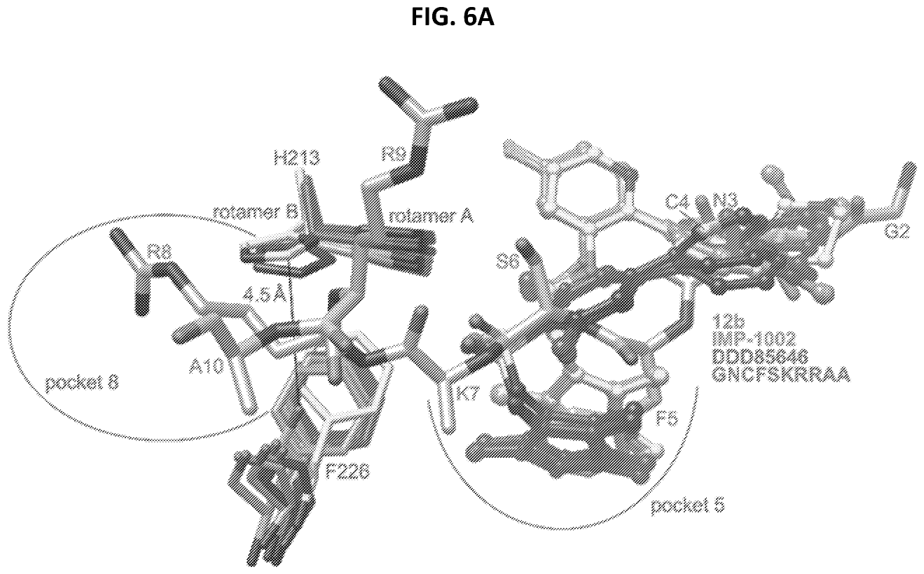

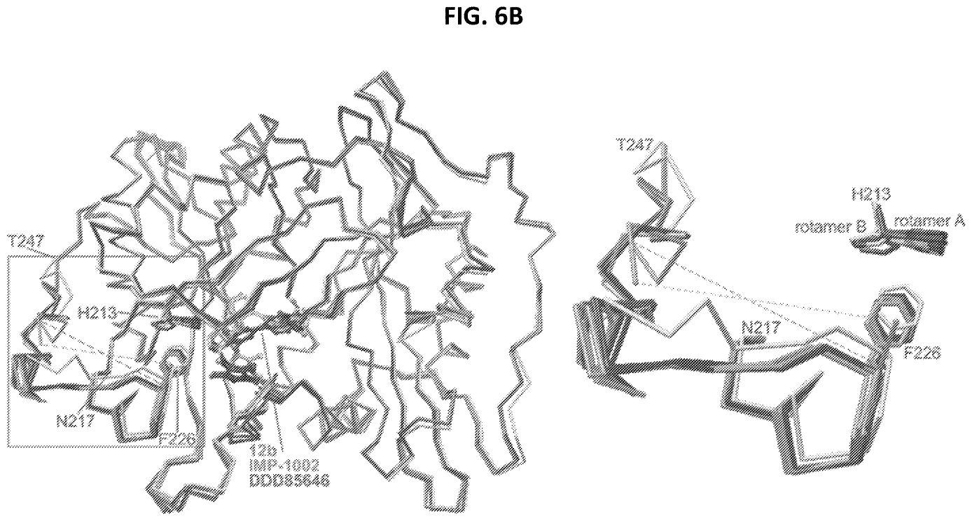

The gain in selectivity achieved via swapping the phenyl and pyridine rings within the core groups of 12b and 12c does not involve changes in polar contacts with PvNMT. Instead, the nitrogen of the pyridine ring of 12b forms a hydrogen bond with a well-ordered water molecule at the opening of a conserved water channel. In addition, the phenyl group, together with the piperazine group, form part of a proximal chloride binding site. Thus, the selectivity of 12b over 12c is predicted to be related to a difference in the water structure surrounding the inhibitors. The structure also shows that 12b induces a displacement in the side chain of Phe226 to a position that sterically favors rotamer B of His213, whereas IMP-1002 binding favors rotamer A (FIG. 2C). Remarkably, the displacement of Phe226 is associated with a major conformational change in residues 217-247, which leads to a narrowing of the substrate binding cleft at pocket 8, with peptide excluding distances separating Phe226 and His213 (FIGS. 6A-6B).

### Evaluation of the Cytotoxicity of the Generated Compounds

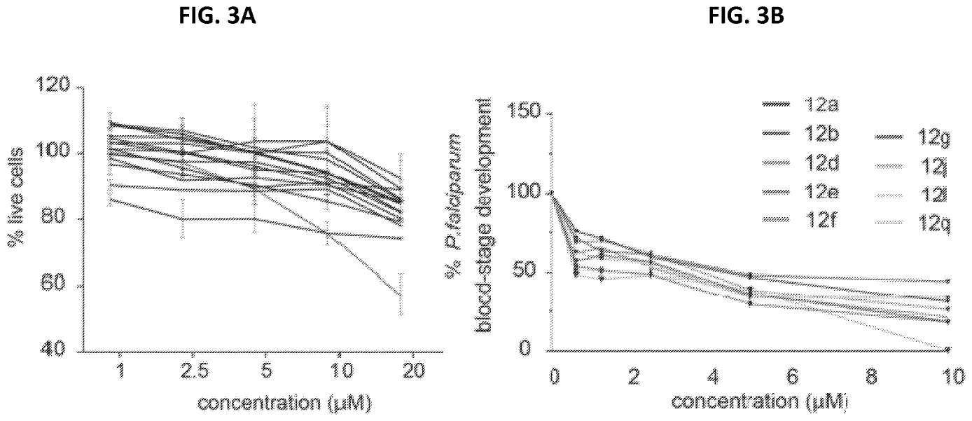

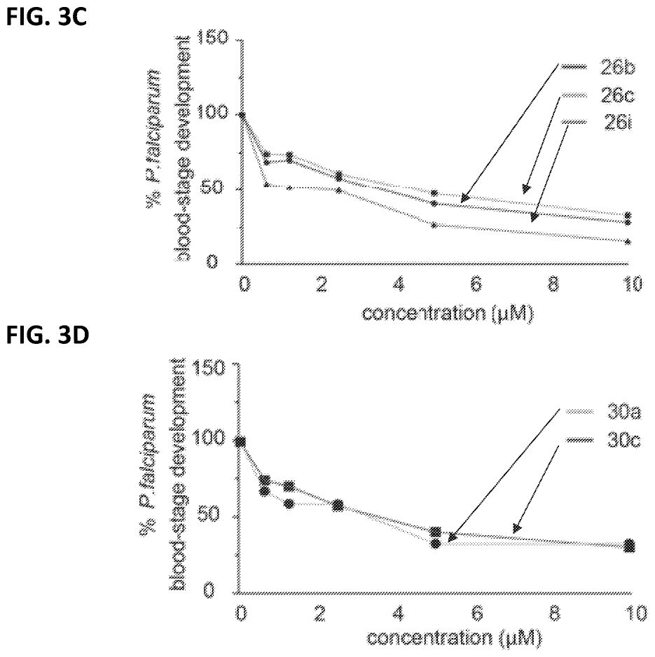

A major concern in developing Plasmodium NMT inhibitors is the potential toxicity in host cells due to cross-reactivity with human NMTs, given the structural overlap between the active site of Plasmodium and human NMTs. Therefore, the cytotoxicity of the compounds was assessed in the human hepatoma HepG2 cell line. The compounds with PvNMT to HsNMT selectivity index above 20 were shortlisted for further screening. HepG2 cells were exposed to the compounds at concentrations ranging from 1 μM to 20 μM for 48 hours, and toxicity was assessed via live-dead staining. Staurosporine, and promiscuous kinase inhibitor and known inducer of cell death in HepG2 cells, was used as a positive control; as expected, it reduced the cell viability at 10-20 μM. None of the NMT-targeting compounds induced >30% cell death in the HepG2 cells, even at their highest concentration of 20 μM (FIG. 3A and Table 3).

### Compounds Show Minimal Toxicity in Human Hepatoma Cells and are Active Against Blood-Stage Parasites

To assess whether any of the NMTis developed here could qualify as TCP-1 candidates, the effect of each compound on blood-stage P. falciparum parasites was assessed, as there is currently no well-established in vitro platform for screening P. vivax blood-stage parasites. Synchronized P. falciparum NF54 ring stage parasites were treated for 72 h with each compound at various concentrations, ranging from 0.625 μM to 10 μM, and parasite DNA replication was measured using a fluorescent DNA binding dye (SYBR Green). The most active compounds in the blood stage had the pyridine moiety in core A and the p-fluorophenyl group in core B in the biaryl scaffold (12e, 12l, and 12i) (FIG. 3B-D). The relative IC₅₀ values for each compound varied from 360 nM to 1.25 μM (Table 3).

TABLE 3
IC₅₀ values of NMT inhibitor compounds for cytotoxicity and
effect on Plasmodium blood and liver stage infection.

| | cytotoxicity | blood stage | liver stage | |
|---|---|---|---|---|
| | HepG2 | Pf NF54 | Pv schizonts | Pv hypnozoites |
| compound | IC₅₀ (μM) | IC₅₀ (nM) | IC₅₀ (μM) | IC₅₀ (μM) |
| 12a | >20 | 810 | 3-3.8 | 5.7 |
| 12b | >20 | 470 | 2.3-4.6 | 1.7 |
| 12d | >20 | 800 | 3.3-4.3 | 4.7 |
| 12e | >20 | 360 | 3.4-3.7 | 1.2 |
| 12f | >20 | 610 | 3.3-4.3 | 1.5 |
| 12g | >20 | 600 | 2.2-3.1 | 1.2 |
| 12j | >20 | 1250 | 4.9-6.0 | 12 |
| 12l | >20 | 370 | 2.9-4.7 | 2.1 |
| 12q | >20 | 380 | 2.2-3.5 | 4.4 |
| 26b | >20 | 710 | 3.1-4.1 | 4.6 |
| 26c | >20 | 820 | 2.8-4.2 | 4.2 |
| 26i | >20 | 400 | 3.3-5.2 | 11 |
| 30a | >20 | 440 | 2.9-4.3 | 2.1 |
| 30c | >20 | 680 | 2.7-4.9 | 3.8 |

### Compounds are Active Against Liver Stage Schizonts

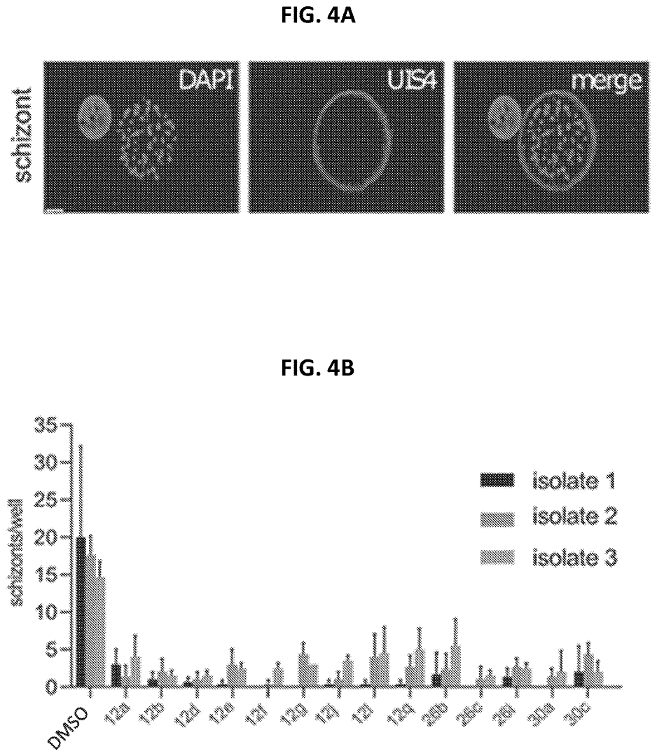

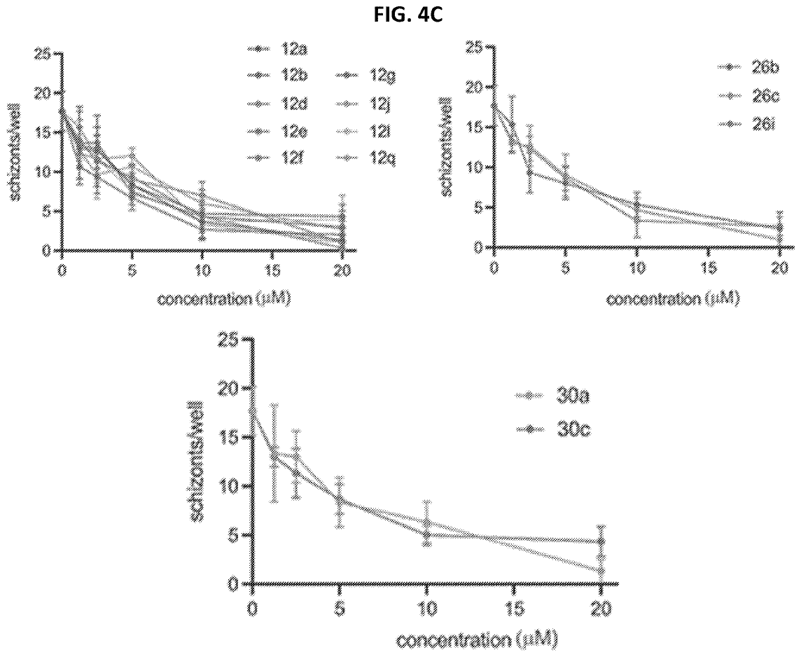

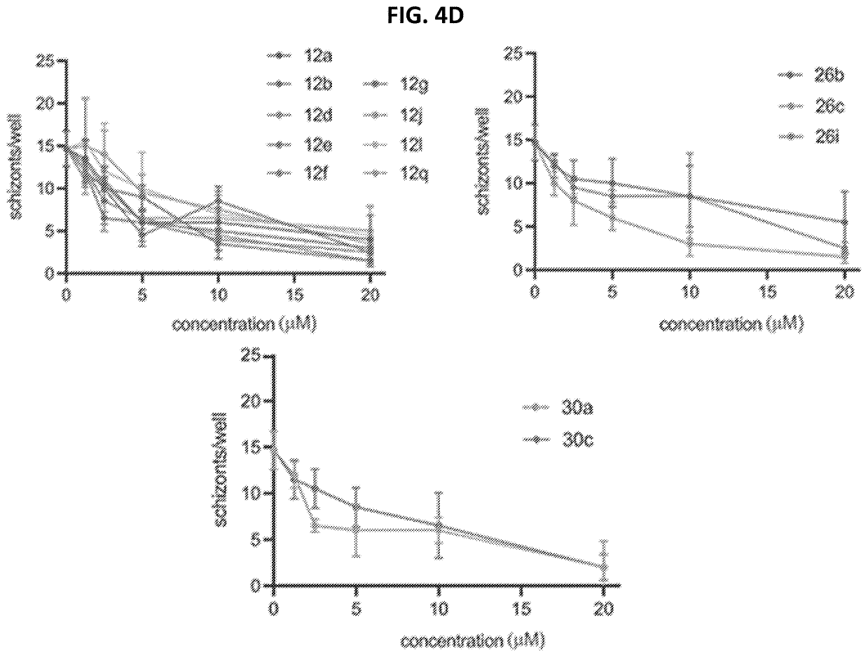

The candidate compounds were next evaluated against liver stage P. vivax (FIG. 4). P. vivax sporozoites were obtained from three independent patient isolates. Liver stage infections were cultured in primary human hepatocytes from a single donor lot using a 384-well microculture system as described previously. Cells were infected and treated with NMTis at 20 μM beginning day 5 post-infection until day 8 when the infection rates were quantified via fluorescent microscopy. Schizonts were defined as any liver stage parasites that exhibited circumferential P. vivax Upregulated in Infectious Sporozoites-4 (PvUIS4) staining, had multiple nuclear masses, and were >10 μm in diameter (FIG. 4A). Robust schizont infections were observed in each of the three independent patient isolates (FIG. 4B), and all the shortlisted compounds reduced the parasite load by at least 90% when administered at 20 μM (FIG. 4B, Table 3). Accordingly, dose-response curves of all the shortlisted NMTis on parasite isolates 3 and 4 were applied over concentrations up to 20 μM (FIGS. 4C and D). Similar kinetics were observed for all inhibitors, with a gradual decrease in parasite load upon increasing the drug concentration. The 8dIC₅₀ values were calculated for each compound and ranged from 2.2 to 6 μM for each compound (Table 3). The most active compounds in this stage (12b and 12g) shared the same scaffold and the piperazine moiety in the tail region, both compounds showed an SI of >150 in the biochemical assay (Table 1).

Compounds are Active Against P. vivax Liver Stage Hypnozoites

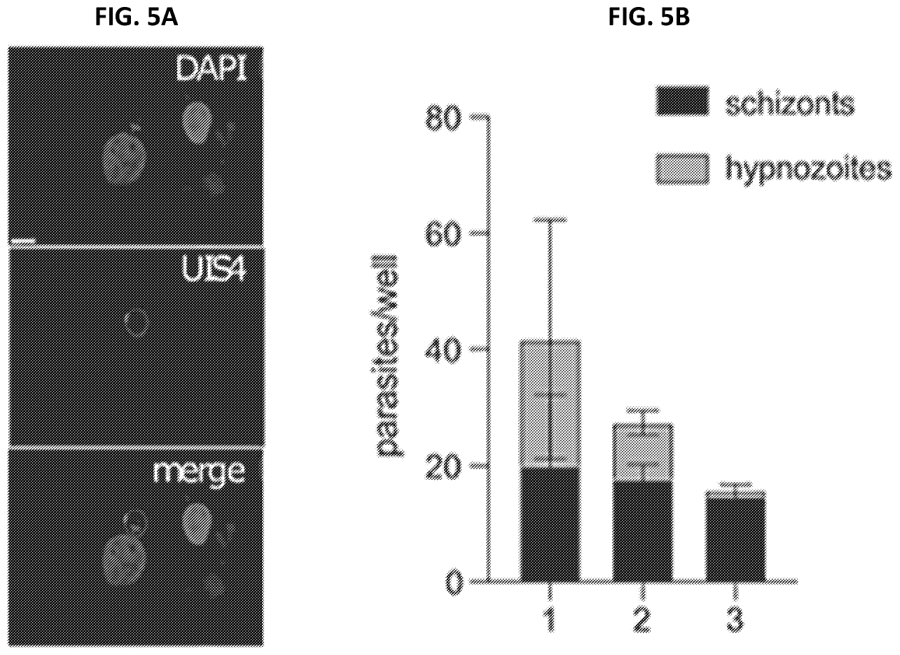

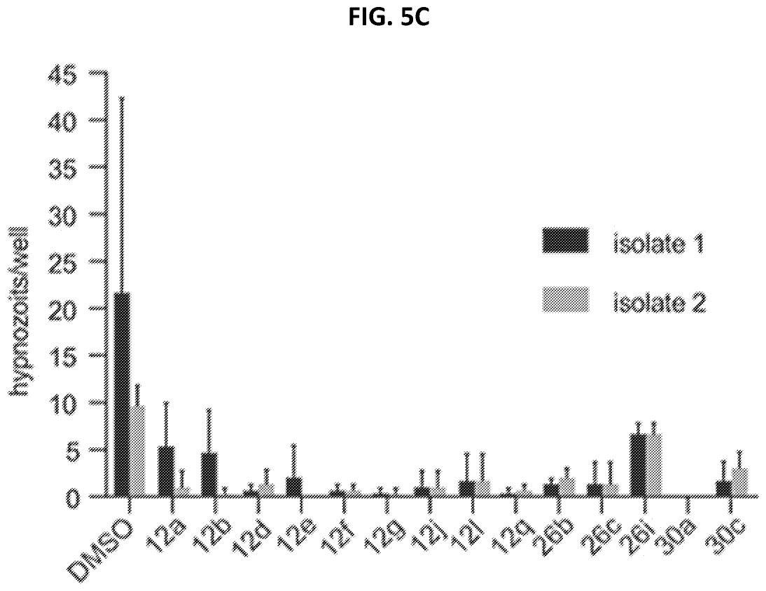

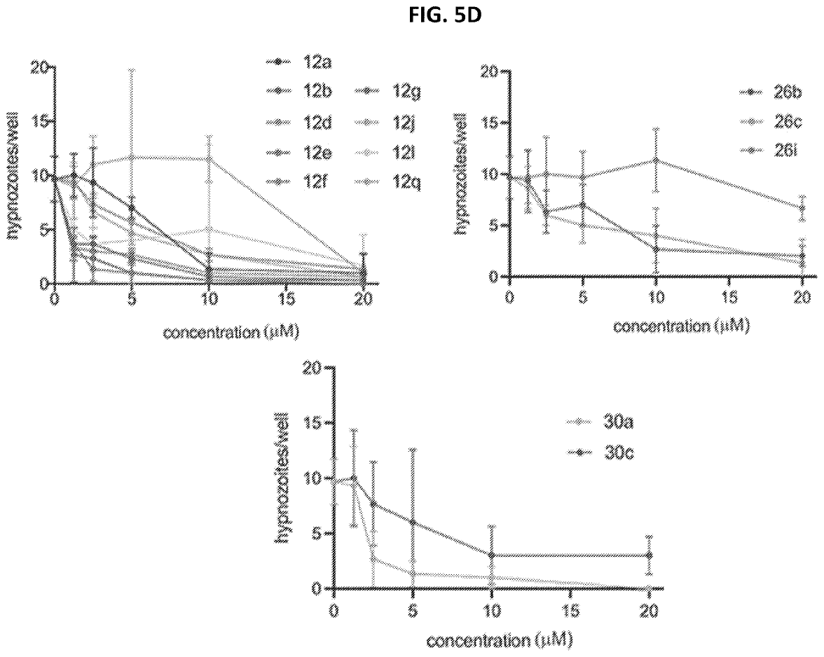

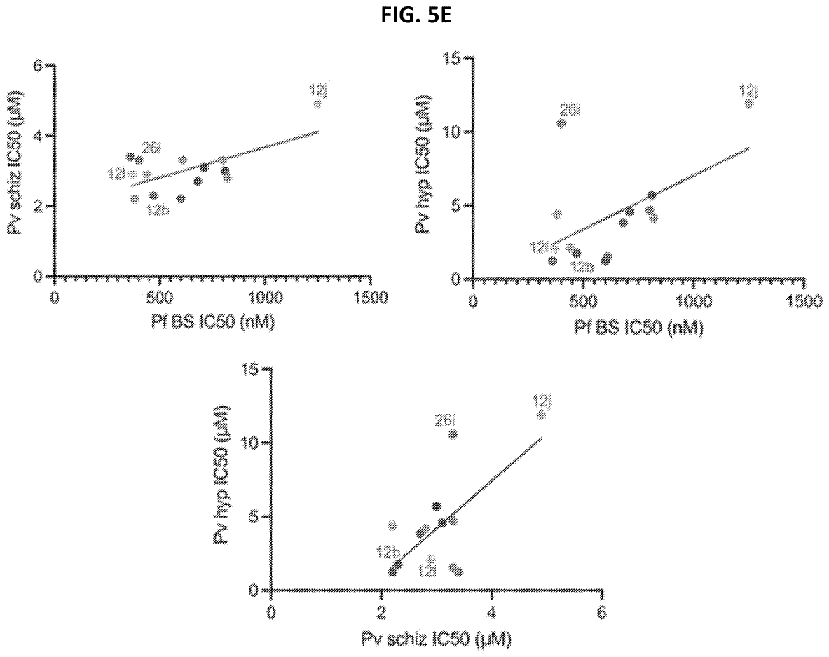

Finally, the NMT-binding compounds were evaluated against non-developing P. vivax hypnozoites. Hypnozoites were defined as parasites having a single nucleus, diameter of <8 mM, and exhibiting prominent staining for PvUIS4 at a point within the parasite periphery (termed the ‘prominence’) (FIG. 5A). Of the three parasite isolates used to infect primary human hepatocytes, two isolates exhibited robust numbers of P. vivax hypnozoites as defined by these criteria (FIG. 5B). Compounds with an SI index>20, except 26i and 30c, exhibited >90% inhibition of hypnozoites at 20 μM (FIG. 5C). Compound 30c exhibited >75% inhibition at 20 μM. The IC₅₀ values were calculated for each compound and ranged from 1.2 to 11.9 μM (FIG. 5D, Table 3). Six compounds showed IC₅₀ values <2.1 μM in this stage, two of which (12b and 30a) had an SI of >250 in the enzymatic assay, both having the same group in the tail and head regions (Table 1 and 2). Interestingly, the efficacy of NMT is on schizont (FIG. 4) and hypnozoite (FIG. 5) P. vivax liver stage forms, as well as efficacy against P. falciparum asexual blood stages (FIG. 3) was strongly correlated (FIG. 5E). Multiple comparison tests for each compound revealed no significant difference in the effect on the schizonts between isolates, except for compound 12q (Table 5). A significant difference in the effect on the hypnozoites between the isolates was observed in compound 26i only. Notably, compound 26i was the only inhibitor that showed a significantly different effect on the two parasite forms, suggesting that moving forward PvNMT targeting compounds might lead to an inhibitor that is effective against multiple life cycle stages of Plasmodium, and across evolved field isolates.

The development of new and potent anti-malarial compounds that target the complete life cycle of Plasmodium, including dormant forms that contribute to relapsing malaria, is needed to eradicate malaria. As most of the currently licensed antimalarials target only the erythrocytic stage, which is responsible for the pathogenesis of the disease, expanding the antimalarial arsenal is crucial. Drugs that can effectively target the liver stage, and especially the dormant forms (hypnozoite) have been a particular challenge to develop. The liver stage is a clinically silent and obligatory developmental phase in the host's liver cells before they can infect erythrocytes and cause malaria symptoms. Targeting the hepatic stage is therefore highly desirable in the context of malaria eradication, not only because its asymptomatic nature makes it ideally suited for prophylactic intervention but also because the liver can serve as a reservoir for P. vivax hypnozoites, the dormant parasite forms that may cause relapses long after the initial blood infection has been treated.

The enzyme NMT is expressed throughout the Plasmodium life cycle and is a promising target for antimalarial drug development. NMT inhibition disrupts at least 3 vital pathways of the parasite lifecycle; during schizont early development, merozoite formation and merozoite egress. Despite this promise, the enzyme has been deprioritized as an anti-malarial target due to challenges in identifying selective inhibitors and a slow MOA. The impact of NMT inhibition on hypnozoites has not been fully investigated. Here it is demonstrated that NMT inhibitors reduce the growth of P. vivax hypnozoites derived from clinical isolates, suggesting that NMT is a target for developing inhibitors with an additional capability of killing dormant forms (hypnozoite) of the parasite lifecycle, an unmet need in the current drug development pipeline. Combination of NMT inhibitors with fast killing blood stage compounds is an attractive strategy for complete parasite elimination.

In this study, a hybrid approach was used to combine previous inhibitor designs to maximize selectivity over the human enzyme. The inhibitors of Formula (I) demonstrated strong selectivity towards PvNMT over HsNMT. A high-resolution cocrystal structure was determined to elucidate the interaction of one of the inhibitors (12b) with the active site of PvNMT. Specifically, it was sought to confirm the mechanism of action, identify the structural determinants associated with the binding of the inhibitors to the active site, and ultimately harness the accumulated structural information and insights gained to further optimize pharmacological activity. The observed pose of 12b in the crystal structure bound to PvNMT validates the design strategy employed. Compound 12b adopts the selective pose displayed by IMP-1002, and the binding of the piperazine moiety near the C-terminal carboxylate largely recapitulates the targeted part of the DDD85646-binding mode. Moreover, the unique features of the binding-site architecture provide a molecular basis for the approximately 100-fold increase in the selectivity of compound 12b over IMP-1002, which can guide future structure-aided drug development.

The compounds described herein can possess one or more stereocenters, and each stereocenter can exist independently in either the (R) or(S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

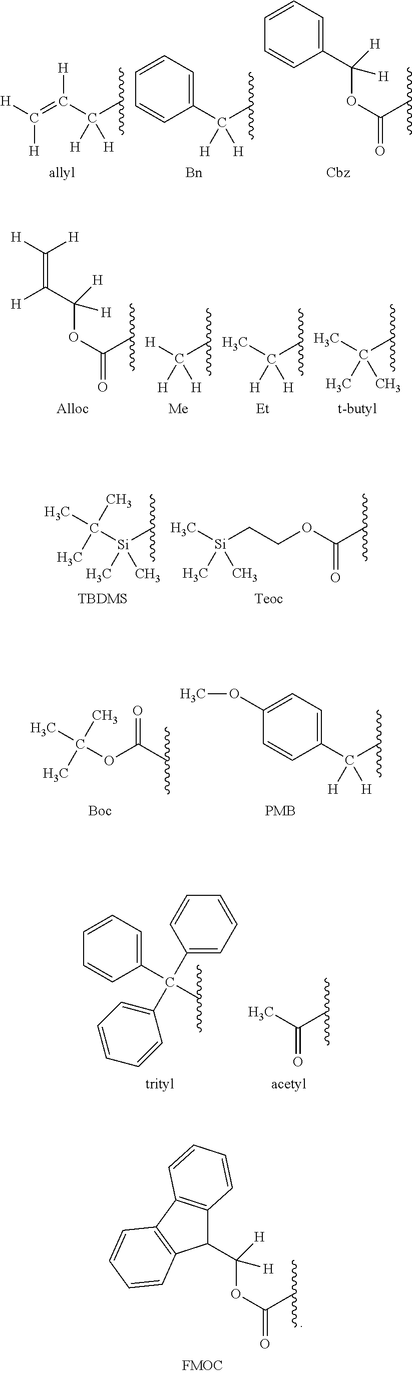

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Pharmacology

In various embodiments, the compound(s) described herein can be administered to a subject in an amount ranging from about 0.01 mg/kg to about 200 mg/kg, or about 0.5 mg/kg to about 190 mg/kg, or about 0.75 mg/kg to about 180 mg/kg, or about 1 mg/kg to about 170 mg/kg, or about 1.5 mg/kg to about 160 mg/kg, or about 2 mg/kg to about 150 mg/kg, or about 2.5 mg/kg to about 140 mg/kg, or about 3 mg/kg to about 130 mg/kg, or about 3.5 mg/kg to about 120 mg/kg, or about 4 mg/kg to about 110 mg/kg, or about 4.5 mg/kg to about 100 mg/kg, or about 5 mg/kg to about 95 mg/kg, or about 5.5 mg/kg to about 90 mg/kg, or about 6 mg/kg to about 85 mg/kg, or about 6.5 mg/kg to about 80 mg/kg, or about 7 mg/kg to about 75 mg/kg, or about 7.5 mg/kg to about 70 mg/kg, or about 8 mg/kg to about 65 mg/kg, or about 8.5 mg/kg to about 60 mg/kg, or about 9 mg/kg to about 55 mg/kg or about 9.5 mg/kg to about 50 mg/kg, or about 10 mg/kg to about 45 mg/kg.

In various embodiments, the compound(s) described herein can be administered to a subject in an amount that is less than, equal to, or greater than about 0.01 mg/kg, 0.05 mg/kg, 0.1 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg. 7 mg/kg, 7.5 mg/kg, 8 mg/kg, 8.5 mg/kg, 9 mg/kg, 9.5 mg/kg, 10 mg/kg, 12 mg/kg, 14 mg/kg, 16 mg/kg, 18 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 100 mg/kg, 105 mg/kg, 110 mg/kg, 115 mg/kg, 120 mg/kg, 125 mg/kg, 130 mg/kg, 140 mg/kg, 145 mg/kg, 150 mg/kg, 155 mg/kg, 160 mg/kg, 170 mg/kg, 175 mg/kg, 180 mg/kg, 185 mg/kg, 190 mg/kg, 195 mg/kg, or 200 mg/kg.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Methods of Treating, Ameliorating, and/or Preventing Parasitic Infections

The disclosure includes a method of treating, preventing, and/or ameliorating a parasitic infection using the compounds of Formula (I). Non-limiting examples of parasitic infections include malaria. In various embodiments, the disclosure includes methods of killing Plasmodium sp. parasitic worms such as Plasmodium falciparum and/or Plasmodium vivax. Surprisingly and unexpectedly, the compounds of Formula (I) can kill even dormant forms of malaria-causing parasites, known as hypnozoites.

In various embodiments, a method of treating, ameliorating, and/or preventing malaria in a subject in need thereof by administering to the subject a therapeutically effective amount of a composition comprising the compound of Formula (I) and at least one pharmaceutically acceptable excipient or carrier. In various embodiments, subject is a human. In various embodiments, the composition is administered to populations known or suspected to be infected with Plasmodium falciparum. In various embodiments, the composition is administered to populations known or suspected to be infected with Plasmodium vivax.

In various embodiments, the administering is by a route selected from the group consisting of oral, buccal, transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

In various embodiments, the subject is infected by Plasmodium falciparum or Plasmodium vivax. In various embodiments, the Plasmodium vivax is in a dormant form (hypnozoites) in the subject. In various embodiments,

In various embodiments, a method of killing or inhibiting a Plasmodium falciparum or Plasmodium vivax parasite is provided. The method of killing or inhibiting includes contacting the parasite with an effective amount of the compound of Formula (I) sufficient to kill the parasite or render the parasite incapable of causing malaria. In various embodiments, the Plasmodium vivax is in a dormant form.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats parasitic infections.

In certain embodiments, administering the compound(s) described herein to the subject allows for administering a lower dose of the additional therapeutic agent as compared to the dose of the additional therapeutic agent alone that is required to achieve similar results in treating a parasitic infection in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating parasitic infections. These additional therapeutic agents may comprise compounds that are commercially available or synthetically accessible to those skilled in the art. These additional therapeutic agents are known to treat or reduce the symptoms, of a parasitic infection.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a parasitic infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a parasitic infections in the patient. An effective amount of the therapeutic compound necessary to achieve a therapeutic effect may vary according to factors such as the state of the disease or disorder in the patient; the age, sex, and weight of the patient; and the ability of the therapeutic compound to treat a parasitic infection in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a parasitic infection in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of oral solutions, oral syrups, or oral suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid). The liquid preparation can contain any of the doses described herein for every 1 mL of the liquid preparation. In various embodiments, the liquid preparation contains the same amount of the compound of Formula (I) as would be administered in a solid oral formulation.

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. patents applications Ser. Nos. 20/030,147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a parasitic infection in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### Materials and Methods

### General Experimental Information for Synthesis and Compound Characterization

General reagents and solvents for synthesizing compounds were purchased from commercial sources and used as supplied unless otherwise stated.

Purification by flash column chromatography was performed on a Selekt (Biotage, U.K.) automated instrument with Sfär KP-amino D or Sfär silica D cartridges (Biotage, U.K.), mobile phase consist of pentane (solvent A) and ethyl acetate (solvent B) or by reverse phase flash column chromatography performed on an Isolera (Biotage, U.K.) automated instrument with Sfär C18 D cartridges (Biotage, U.K.), mobile phase consisting of water (solvent A) and acetonitrile (solvent B). The standard gradient consisted of x % solvent B for one column volume, x % to y % B for 10 column volumes, and then y % B for 2 column volumes. x and y are defined in the characterization section of the compound the interest.

All NMR spectra (¹H and ¹³C) were recorded on a Varian 400 MHz spectrometer at 25° C. Samples were dissolved (0.5 mL) in deuterated chloroform, methanol, or dimethylsulfoxide (CDCl₃, CD₃OD, DMSO-d₆). The residual solvent peaks specific to the deuterated solvent was used as an internal reference; CDCl₃: 7.26 ppm (¹H NMR) and 77.20 ppm (¹³C NMR); CD₃OD: 3.31 ppm (¹H NMR) and 49.00 ppm (¹³C NMR); DMSO-d₆: 2.50 ppm (¹H NMR) and 39.52 ppm (¹³C NMR). Data are presented as follows: chemical shift in ppm, multiplicity (br=broad, s=singlet, d=doublet, dd=doublet of doublet, t=triplet, q=quartet, m=multiplet), coupling constants in Hz and integration.

The purity of selected compounds was performed in analytical HPLC (Waters 2690 Separations Module; Atlantis® T3, 5 μm column, 4.6×250 mm; H₂O/ACN (0.1% TFA)). High-resolution mass spectra (HRMS) were recorded on an Agilent 1290 infinity LC system in tandem with an Agilent 6520 Accurate Mass Q-TOF spectrometer.

### Detailed Synthetic Procedure and Characterization of Compounds

### General Procedure A—Reduction of Ester to 1° Alcohol

To a solution of lithium aluminum hydride (2.5 equiv.) in anhydrous tetrahydrofuran (15 mL) cooled to 0° C. was added slowly a solution of the selected ester (1 equiv.) in anhydrous tetrahydrofuran (25 mL). The mixture was stirred a room temperature for 12 h. Then the reaction mixture was cooled to 0° C., and 2 mL of water was slowly added and stirred for 10 min. The reaction was filtered off in the presence of celite and rinsed with EtOAc (2×50 mL) the residue was concentrated under reduced pressure to afford the desired alcohol.

### General Procedure B—Mesyl Transfer

The solution of selected alcohol (1 equiv.) and NEt₃ (1.5 equiv.) in DCM (10 mL) was added to methanesulfonyl chloride (1.2 equiv.) at 0°−5° C. The mixture was stirred at room temperature for 3 h. After completion of the reaction, the mixture was quenched in water, and the organic compound was extracted by DCM. The organic layer was dried over Na₂SO₄, and the solvent was removed under reduced pressure to afford the desired phenylmethane sulfonate. Next, A solution of the appropriate phenylmethane sulfonate (2 equiv.) in dry acetonitrile (3 mL) in a microwave vial (2-5 mL) was treated with a solution of the appropriate alcohol (1 equiv.), followed by solid sodium t-butoxide (1.2 equiv.). The vial was sealed and then heated under microwave irradiation to 140° C. for 30 min. The reaction mixture was partitioned between EtOAc (20 mL) and saturated sodium carbonate solution (10 mL). The organic phase was dried over Na₂SO₄, and concentrated under reduced pressure and the crude product was purified by flash column chromatography by elution with Pentane/EtOAc in gradient to give the desired aryl-ether.

General procedure C-Mitsunobu reaction

A solution of selected alcohol (1 equiv.) in toluene (10 mL) was reacted with selected aryl or pyridine-alcohol (1.25 equiv.) and cyanomethyiene tributylphosphorane (1.5 equiv.) at 100° C. for 16 h. The reaction mixture was then diluted with ethyl acetate (50 mL) and washed with water (1×25 mL) and brine (2×25 mL), The organic phase was dried over Na₂SO₄, concentrated under reduced pressure, and the crude product was purified by flash column chromatography by elution with Pentane/EtOAc or by reverse-phase chromatography using H₂O/ACN in gradient in gradient to give the desired aryl-ether.

### General Procedure D—Suzuki Cross-Coupling Reaction

A solution of selected aryl-bromide (1 equiv.) was dissolved in 1,4-dioxane (10 mL) in a microwave vial (10-20 mL) and treated with the corresponding aryl boronic acid pinacol ester (1.25 equiv.) and tetrakis(triphenylphosphine) palladium (0) (0.05 equiv.), followed by a solution of potassium phosphate (2.7 equiv.) in water (3 mL) under N₂. The reaction mixture was heated at 100° C. for 1-3 h in a heating plate. The resulting solution was cooled to room temperature and evaporated under reduced pressure. The residue was partitioned between EtOAc (20 mL) and saturated sodium bicarbonate solution (20 mL). The organic phase was dried over Na₂SO₄, and concentrated under reduced pressure and the crude product was purified by flash column chromatography by elution with Pentane/EtOAc in gradient or by reverse-phase chromatography using H₂O/ACN in gradient to give the desired compound.

### General Procedure E—Boc-Deprotection

The Boc-protected amine was dissolved in dioxane (1 mL) and treated with a solution of HCl in dioxane (4 M, 2 mL). The reaction mixture was stirred at room temperature overnight. All volatiles were removed under reduced pressure, and the product was triturated with ether and DCM, redissolved in water, and freeze-dried to afford the desired.

### 2-(1,3,5-Trimethyl-1H-pyrazol-4-yl)ethan-1-ol (1)

Ethyl 3-acetyl-4-oxopentanoate (1a) and Ethyl 2-(1,3,5-trimethyl-1H-pyrazol-4-yl) acetate (1b) were prepared as described previously (14). Compound 1b was reacted according to General Procedure A. Afforded the title compound as a colorless oil (85.1%). ¹H NMR (CDCl₃, 400 MHz, δ, ppm) 3.68 (3H, s), 3.63 (2H, t, J=6.8 Hz), 2.59 (2H, t, J=6.8 Hz), 2.16 (3H, s), 2.15 (3H, s).

### 2-(3,5-Dimethyl-1H-pyrazol-4-yl)ethan-1-ol (2)

Ethyl 3-acetyl-4-oxopentanoate (la) and Ethyl 2-(3,5-dimethyl-1H-pyrazol-4-yl)acetate (2b) were prepared as described previously (14). Compound 2b was reacted according to General Procedure A. Afforded the title compound as a colorless oil (80.1%). ¹H NMR (CD₃OD, 400 MHz, δ, ppm) 3.52 (2H, t, J=7.2 Hz), 2.55 (2H, t, J=7.2 Hz), 2.14 (6H, s).

### 4-(2-(2-bromophenoxy) ethyl)-1,3,5-trimethyl-1H-pyrazole (3)

Following general procedure B, 2-bromophenol (250 μL, 2.15 mmol) was reacted with mesyl-chloride (208 μL, 2.69 mmol) and NEt₃ (450 μL, 3.23 mmol) to afford 500 mg (92%) of 2-bromophenyl methanesulfonate. ¹H NMR (CDCl₃, 400 MHz, δ, ppm) 7.64 (1H, dd, J=8.0, 1.6 Hz), 7.47 (1H, dd, J=8.2, 1.6 Hz), 7.37 (1H, ddd, J=8.2, 7.4, 1.6 Hz), 7.20 (1H, ddd, J=8.0, 7.4, 1.6 Hz), 2.27 (3H, s). Next, 2-bromophenyl methanesulfonate (488 mg, 1.94 mmol) was reacted with 1 (150 mg, 0.97 mmol) to afford 122 mg (40%) of the title compound as a yellow oil. ¹H NMR (CDCl₃, 400 MHz, δ, ppm) 7.52 (1H, dd, J=7.8, 1.6 Hz), 7.22 (1H, ddd, J=8.3, 7.4, 1.6 Hz), 6.85-6.78 (2H, m), 4.00 (2H, t, J=7.0 Hz), 3.71 (3H, s), 2.89 (2H, t, J=7.0 Hz), 2.23 (6H, s).

### 2-bromo-3-(2-(1,3,5-trimethyl-1H-pyrazol-4-yl)ethoxy)pyridine (4)

Following general procedure C, 1 (100 mg, 0.71 mmol) was reacted with 2-bromo-3-hydroxypyridine (155 mg, 0.89 mmol). The crude was purified by flash column chromatography (Pentane/EtOAc in gradient) to afford 145 mg (63%) of the title compound as a yellow pale solid. ¹H NMR (CDCl₃, 400 MHz, δ, ppm) 7.87 (1H, dd, J=4.6, 1.6 Hz), 7.11 (1H, ddd, J=8.1, 4.6, 0.9 Hz), 7.01 (1H dd, J=8.1, 1.5 Hz), 3.95 (2H, t, J=6.8 Hz), 3.62 (3H, s), 2.84 (2H, t, J=6.6 Hz,), 2.18 (3H, s), 2.16 (3H, s).

### 4-(2-(2-bromo-5-fluorophenoxy) ethyl)-1,3,5-trimethyl-1H-pyrazole (5)

Following general procedure B, 2-bromo-5-fluorophenol (250 μL, 2.24 mmol) was reacted with mesyl-chloride (217 μL, 2.80 mmol) and NEt₃ (470 μL, 3.37 mmol) to afford 530 mg (87%) of 2-bromo-5-fluorophenyl methanesulfonate. ¹H NMR (CDCl₃, 400 MHz, δ, ppm) 7.61 (1H, dd, J=8.9, 5.7 Hz), 7.25 (1H, dd, J=8.7, 2.9 Hz), 6.97 (1H, ddd, J=8.9, 7.6, 2.9 Hz), 3.29 (3H, s). Next, 2-bromo-5-fluorophenyl methanesulfonate (500 mg, 1.85 mmol) was reacted with 1 (143 mg, 0.92 mmol) to afford 113 mg (37%) of the title compound as a yellow oil. ¹H NMR (CDCl₃, 400 MHz, δ, ppm) 7.49-7.42 (1H, m), 6.60-6.53 (2H, m), 3.96 (2H, t, J=6.9 Hz), 3.71 (3H, s), 2.89 (2H, t, J=6.9 Hz,), 2.24 (3H, s), 2.23 (3H, s).

### 4-(2-(2-bromophenoxy) ethyl)-3,5-dimethyl-1H-pyrazole (6)

Following general procedure C, 2 (100 mg, 0.71 mmol) was reacted with 2-bromophenol (103 μL, 0.89 mmol). The crude was purified by flash column chromatography (Pentane/EtOAc in gradient) to afford 130 mg (61%) of the title compound as a yellow pale solid. ¹H NMR (CDCl₃, 400 MHz, δ, ppm) 7.51 (1H, dd, J=7.8, 1.6 Hz), 7.21 (1H, ddd, J=8.3, 7.4, 1.6 Hz), 6.84-6.78 (2H, m), 4.00 (2H, t, J=7.1 Hz), 2.90 (2H, t, J=7.1 Hz), 2.28 (6H, s).

### 2-bromo-3-(2-(3,5-dimethyl-1H-pyrazol-4-yl)ethoxy)pyridine (7)

Following general procedure C, 2 (100 mg, 0.71 mmol) was reacted with 2-bromo-3-hydroxypyridine (155 mg, 0.89 mmol). The crude was purified by flash column chromatography (Pentane/EtOAc in gradient) to afford 140 mg (66%) of the title compound as a yellow pale solid. ¹H NMR (CDCl₃, 400 MHz, δ, ppm) 7.96 (1H, dd, J=4.6, 1.6 Hz), 7.18 (1H, dd, J=8.1, 4.6 Hz), 7.06 (1H, dd, J=8.1, 1.6 Hz), 4.03 (2H, t, J=6.8 Hz), 2.93 (2H, t, J=6.8 Hz,), 2.29 (s, 6H).

4-(2-(2-bromo-5-fluorophenoxy) ethyl)-3,5-dimethyl-1H-pyrazole (8)

Following general procedure C, 2 (78 mg, 0.55 mmol) was reacted with 2-bromo-5-fluorophenol (80 μL, 0.71 mmol). The crude was purified by flash column chromatography (Pentane/EtOAc in gradient) to afford 120 mg (68%) of the title compound as a yellow pale solid. ¹H NMR (CDCl₃, 400 MHz, δ, ppm) 7.44 (1H, dd, J=8.8, 6.2 Hz), 6.60-6.52 (2H, m), 3.98 (2H, t, J=6.9 Hz), 2.91 (2H, t, J=6.9 Hz), 2.30 (6H, s).

### 3-(2-(2-bromophenoxy) ethyl)pyridine (9)

Following general procedure B, 2-bromophenyl methanesulfonate (500 mg, 1.99 mmol) was reacted with 3-(2-hydroxyethyl)pyridine (112 μL, 0.99 mmol) to afford 125 mg (45%) of the title compound as a yellow solid. ¹H NMR (CDCl₃, 400 MHz, δ, ppm) 8.61 (1H, d, J=2.1 Hz), 8.50 (1H, dd, J=4.8, 1.6 Hz), 7.78-7.72 (1H, m), 7.52 (1H, dd, J=7.8, 1.6 Hz), 7.27-7.24 (2H, m), 7.24-7.21 (1H, m), 6.87-6.79 (2H, m), 4.22 (2H, t, J=6.3 Hz), 3.15 (2H, t, J=6.3 Hz).

### 2-bromo-3-(2-(pyridin-3-yl)ethoxy)pyridine (10)

Following general procedure C, 3-(2-hydroxyethyl)pyridine (112 μL, 0.99 mmol) was reacted with 2-Bromo-3-hydroxypyridine (138 mg, 0.79 mmol). The crude was purified by flash column chromatography (Pentane/EtOAc in gradient) to afford 118 mg (69%) of the title compound as a yellow pale solid. ¹H NMR (CDCl₃, 400 MHz, δ, ppm) 8.61 (1H, d, J=2.3 Hz), 8.52 (1H, dd, J=4.8, 1.6 Hz), 7.98 (1H, dd, J=4.6, 1.6 Hz), 7.76 (1H, dt, J=7.8, 2.0 Hz), 7.27 (2H, dd, J=7.8, 4.8 Hz), 7.18 (1H, ddd, J=8.1, 4.6, 0.4 Hz), 7.08 (1H, dd, J=8.1, 1.5 Hz), 4.22 (2H, t, J=6.3 Hz), 3.18 (2H, t, J=6.3 Hz).

### 3-(2-(2-bromo-5-fluorophenoxy) ethyl)pyridine (11)

Following general procedure C, 3-(2-hydroxyethyl)pyridine (68 μL, 0.60 mmol) was reacted with 2-Bromo-5-fluorophenol (84 μL, 0.75 mmol). The crude was purified by flash column chromatography (Pentane/EtOAc in gradient) to afford 130 mg (72%) of the title compound as a yellow pale solid. ¹H NMR (CDCl₃, 400 MHz, δ, ppm) 8.55 (1H, d, J=2.2 Hz), 8.44 (1H, dd, J=4.8, 1.6 Hz), 7.71-7.63 (1H, m), 7.37 (1H, ddd, J=8.0, 6.2, 0.7 Hz), 7.18 (1H, ddd, J=7.8, 4.8, 0.7 Hz), 6.55-6.45 (2H, m), 4.09 (2H, t, J=6.2 Hz), 3.07 (2H, t, J=6.2 Hz).

### 2′-(piperazin-1-yl)-3-(2-(1,3,5-trimethyl-1H-pyrazol-4-yl)ethoxy)-2,4′-bipyridine (12a)

Following general procedure D, 4 (35 mg, 0.11 mmol) was reacted with 2-(4-tert-butoxycarbonylpiperazin-1-yl)pyridine-4-boronic acid, pinacol ester (51 mg, 0.14 mmol), the crude was purified by reverse phase chromatography (6 g C18 column; MeCN in water 0-100%). The resulting residue was reacted according to general procedure E. Afforded the title compound as a fluffy colorless solid (7 mg, 16%). ¹H NMR (CD₃OD, 400 MHz, δ, ppm) 8.20 (1H dd, J=4.7, 1.3 Hz), 8.13 (1H, dd, J=5.3, 0.7 Hz), 7.57 (1H, dd, J=8.5, 1.3 Hz), 7.41 (1H, dd, J=8.5, 4.7 Hz), 7.12-7.11 (1H, m), 6.95 (1H, dd, J=5.3, 1.3 Hz), 4.15 (2H, 1, J=6.5 Hz), 3.63 (3H, s), 3.54-3.50 (4H, m), 2.97-2.93 (4H, m), 2.83 (2H, t, J=6.5 Hz), 2.05 (3H, s), 2.01 (3H, s). ¹³C NMR (CD₃OD, 100 MHz, δ, ppm) 161.35, 154.85, 148.38, 148.12, 147.57, 146.90, 141.92, 139.20, 125.97, 122.31, 115.31, 113.29, 109.38, 69.80, 47.34, 46.31, 35.77, 24.38, 11.61, 9.40. HRMS (ESI), found 393.2403 C₂₂H₂₈N₆O, [M+H]⁺, requires 393.2403.

### 1-(3-(3-(2-(1,3,5-trimethyl-1H-pyrazol-4-yl)ethoxy)pyridin-2-yl)phenyl)piperazine (12b)

Following general procedure D, 4 (100 mg, 0.32 mmol) was reacted with (3-(4-(tert-butoxycarbonyl)piperazin-1-yl)phenyl)boronic acid (123 mg, 0.40 mmol), the crude was purified by reverse phase chromatography (6 g C18 column; MeCN in water 0-100%). The resulting residue was reacted according to general procedure E. Afforded the title compound as a fluffy yellow solid (70 mg, 83%). ¹H NMR (CD₃OD, 400 MHz, δ, ppm) 8.40 (1H, dd, J=5.7, 1.1 Hz), 8.33 (1H, dd, J=8.8, 1.0 Hz), 7.99 (1H, dd, J=8.8, 5.7 Hz), 7.51 (1H, t, J=7.9 Hz), 7.38-7.32 (2H, m), 7.18-7.14 (1H, m), 4.39 (2H, t, J=6.0 Hz), 3.86 (3H, s), 3.56 (4H, dd, J=6.3, 4.1 Hz), 3.41 (4H, dd, J=6.3, 4.1 Hz), 2.99 (2H, t, J=6.0 Hz), 2.15 (3H, s), 2.11 (3H, s). ¹³C NMR (CD₃OD, 100 MHz, δ, ppm) 156.36, 152.02, 146.03, 145.13, 144.75, 134.15, 131.19, 131.16, 130.54, 128.24, 122.85, 120.55, 118.55, 116.47, 70.77, 47.26, 44.63, 35.58, 23.24, 9.59, 9.33. HRMS (ESI), found 392.2454 C₂₃H₂₉N₅O, [M+H]⁺, requires 392.2453.

### 1-(4-(2-(2-(1,3,5-trimethyl-1H-pyrazol-4-yl)ethoxy)phenyl)pyridin-2-yl)piperazine (12c)

Following general procedure D, 3 (100 mg, 0.32 mmol) was reacted with 2-(4-tert-butoxycarbonylpiperazin-1-yl)pyridine-4-boronic acid, pinacol ester (154 mg, 0.40 mmol), the crude was purified by reverse phase chromatography (6 g C18 column; MeCN in water 0-100%). The resulting residue was reacted according to general procedure E. Afforded the title compound as a fluffy pale yellow solid (65 mg, 51%). ¹H NMR (CD₃OD, 400 MHz, δ, ppm) 8.02 (1H, dd, J=5.3, 0.6 Hz), 7.27 (1H, ddd, J=8.3, 7.4, 1.8 Hz), 7.22 (1H, dd, J=7.6, 1.8 Hz), 7.00-6.97 (1H, m), 6.95 (1H, td, J=7.6, 1.1 Hz), 6.80 (1H, s), 6.66 (1H, dd, J=5.3, 1.1 Hz), 3.97 (2H, t, J=6.5 Hz), 3.57 (3H, s), 3.45-3.40 (4H, m), 2.90-2.86 (4H, m), 2.69 (2H, t, J=6.5 Hz), 1.98 (3H, s), 1.91 (3H, s). ¹³C NMR (CD₃OD, 100 MHz, δ, ppm) 161.29, 157.12, 150.22, 147.85, 146.79, 139.06, 131.36, 130.90, 130.31, 122.10, 116.19, 113.94, 113.58, 109.72, 69.57, 47.41, 46.33, 35.75, 24.56, 11.65, 9.38. HRMS (ESI), found 392.2451 C₂₃H₂₉N₅O, [M+H]⁺, requires 392.2450.

### 1-(2′-(2-(1,3,5-trimethyl-1H-pyrazol-4-yl)ethoxy)-[1,1′-biphenyl]-3-yl)piperazine (12d)

Following general procedure D, 3 (100 mg, 0.32 mmol) was reacted with (3-(4-(tert-butoxycarbonyl)piperazin-1-yl)phenyl)boronic acid (123 mg, 0.40 mmol), the crude was purified by reverse phase chromatography (6 g C18 column; MeCN in water 0-100%). The resulting residue was reacted according to general procedure E. Afforded the title compound as a fluffy brownish white solid (60 mg, 58%). ¹H NMR (CD₃OD, 400 MHz, δ, ppm) 7.34-7.25 (2H, m), 7.18 (1H, dd, J=7.5, 1.7 Hz), 7.14-7.10 (2H, m), 7.05-7.02 (1H, m), 6.98 (1H, td, J=7.5, 0.8 Hz), 6.93 (1H, d, J=7.7 Hz), 4.09 (2H, t, J=5.8 Hz), 3.84 (3H, s), 3.54 (4H, dd, J=6.4, 3.4 Hz), 3.45 (4H, dd, J=6.4, 3.3 Hz), 2.86 (2H, t, J=5.8 Hz), 2.09 (3H, s), 2.06 (3H, s). ¹³C NMR (CD₃OD, 100 MHz, δ, ppm) 156.83, 150.29, 146.30, 144.45, 141.88, 132.43, 131.87, 130.10, 130.08, 124.93, 122.35, 119.89, 117.68, 117.11, 114.03, 68.77, 48.68, 44.51, 35.49, 23.68, 9.40, 9.22. HRMS (ESI), found 391.2499 C₂₄H₃₀N₄O, [M+H]⁺, requires 391.2498.

### 1-(4-(4-fluoro-2-(2-(1,3,5-trimethyl-1H-pyrazol-4-yl)ethoxy)phenyl)pyridin-2-yl)piperazine (12e)

Following general procedure D, 5 (50 mg, 0.15 mmol) was reacted with 2-(4-tert-butoxycarbonylpiperazin-1-yl)pyridine-4-boronic acid, pinacol ester (75 mg, 0.19 mmol), the crude was purified by reverse phase chromatography (6 g C18 column; MeCN in water 0-100%). The resulting residue was reacted according to general procedure E. Afforded the title compound as a fluffy pale yellow solid (11 mg, 52%). ¹H NMR (CD₃OD, 400 MHz, δ, ppm) 8.11 (1H, dd, J=5.2, 0.7 Hz), 7.28 (1H, dd, J=8.5, 6.7 Hz), 6.89-6.84 (2H, m), 6.77-6.71 (2H, m), 4.05 (2H, t, J=6.4 Hz), 3.79-3.73 (4H, m), 3.31-3.27 (4H, m), 3.61 (3H, s), 2.76 (2H, t, J=6.3 Hz), 1.97 (3H, s), 1.96 (3H, s). ¹³C NMR (CD₃OD, 100 MHz, δ, ppm) 165.14 (d, J=245 Hz), 160.06, 158.50 (d, J=10.1 Hz), 149.79, 148.24, 146.94, 139.15, 132.49 (d, J=10.1 Hz), 126.16 (d, J=3.3 Hz), 117.36, 113.54, 109.96, 108.25 (d, J=21.5 Hz), 101.70 (d, J=25.90 Hz), 69.89, 44.53, 44.13, 35.76, 24.29, 11.56, 9.38. HRMS (ESI), found 410.2356 C₂₃H₂₈FNO, [M+H]⁺, requires 410.2356.

### 1-(4′-fluoro-2′-(2-(1,3,5-trimethyl-1H-pyrazol-4-yl)ethoxy)-[1,1′-biphenyl]-3-yl)piperazine (12f)

Following general procedure D, 5 (100 mg, 0.30 mmol) was reacted with (3-(4-(tert-butoxycarbonyl)piperazin-1-yl)phenyl)boronic acid (121 mg, 0.39 mmol), the crude was purified by reverse phase chromatography (6 g C18 column; MeCN in water 0-100%). The resulting residue was reacted according to general procedure E. Afforded the title compound as a fluffy brownish white solid (60 mg, 58%). ¹H NMR (CD₃OD, 400 MHz, δ, ppm) 7.31-7.26 (1H, m), 7.17 (1H, dd, J=8.4, 6.8 Hz), 7.04 (1H, ddd, J=8.2, 2.5, 0.8 Hz), 7.00-6.97 (1H, m), 6.88-6.81 (2H, m), 6.72 (1H, td, J=8.3, 2.5 Hz), 4.09 (2H, t, J=5.8 Hz), 3.84 (3H, s), 3.47 (4H, dd, J=6.7, 3.5 Hz), 3.40 (4H, dd, J=6.6, 3.6 Hz), 2.87 (2H, 1, J=5.8 Hz), 2.08 (3H, s), 2.05 (3H, s). ¹³C NMR (CD₃OD, 100 MHz, δ, ppm) 164.40 (d, J=243.3), 158.05 (d, J=9.9), 151.43, 146.24, 144.53, 140.81, 132.73 (d, J=9.9), 130.04, 128.74 (d, J=3.3), 123.86, 119.42, 117.45, 116.77, 108.27 (d, J=21.2), 101.69 (d, J=25.85), 69.06, 48.03, 44.76, 35.46, 23.48, 9.37, 9.17. HRMS (ESI), found 429.2407 C₂₄H₃₀FN₄O, [M+H]⁺, requires 429.2404.

### 1-(3-(3-(2-(pyridin-3-yl)ethoxy)pyridin-2-yl)phenyl)piperazine (12g)

Following general procedure D, 10 (100 mg, 0.35 mmol) was reacted with (3-(4-(tert-butoxycarbonyl)piperazin-1-yl)phenyl)boronic acid (137 mg, 0.44 mmol), the crude was purified by reverse phase chromatography (6 g C18 column; MeCN in water 0-100%). The resulting residue was reacted according to general procedure E. Afforded the title compound as a fluffy yellow solid (70 mg, 83%). ¹H NMR (CD₃OD, 400 MHz, δ, ppm) 8.73 (2H, s), 8.41 (2H, d, J=7.4 Hz), 8.37-8.32 (1H, m,), 7.98 (1H, dd, J=8.7, 5.7 Hz,), 7.95-7.88 (1H, m), 7.51-7.45 (1H, m), 7.33-7.28 (2H, m), 7.15-7.10 (1H, m), 4.63 (2H, t, J=6.0 Hz), 3.54 (4H, dd, J=6.3, 4.0 Hz), 3.44-3.37 (6H, m). ¹³C NMR (CD₃OD, 100 MHz, δ, ppm) 154.49, 150.46, 147.37, 143.83, 141.36, 139.58, 133.29, 129.64, 129.49, 128.99, 126.67, 121.56, 119.16, 117.19, 69.05, 45.85, 43.23, 31.48. HRMS (ESI), found 361.2029 C₂₂H₂₄N₄O, [M+H]⁺, requires 361.2028.

### 2′-(piperazin-1-yl)-3-(2-(pyridin-3-yl)ethoxy)-2,4′-bipyridine (12h)

Following general procedure D, 10 (88 mg, 0.31 mmol) was reacted with 2-(4-tert-butoxycarbonylpiperazin-1-yl)pyridine-4-boronic acid, pinacol ester (153 mg, 0.39 mmol), the crude was purified by reverse phase chromatography (6 g C18 column; MeCN in water 0-100%). The resulting residue was reacted according to general procedure E. Afforded the title compound as a fluffy yellow solid (40 mg, 85%). ¹H NMR (CD₃OD, 400 MHz, δ, ppm) 8.81 (1H, s), 8.75 (1H, d, J=5.5 Hz), 8.60 (1H, d, J=8.1 Hz), 8.39 (1H, d, J=4.5 Hz), 8.15 (1H, d, J=6.4 Hz), 8.03 (1H, dd, J=8.0, 5.9 Hz), 7.94 (1H, dd, J=8.7, 0.9 Hz), 7.79-7.76 (1H, m), 7.70 (1H, dd, J=8.6, 4.9 Hz), 7.44 (1H, dd, J=6.4, 1.2 Hz), 4.58 (2H, t, J=6.2 Hz), 4.09-4.03 (4H, m), 3.51-3.46 (4H, m), 3.44 (2H, t, J=6.2 Hz). ¹³C NMR (CD₃OD, 100 MHz, δ, ppm) 155.73, 154.62, 148.91, 142.74, 142.18, 141.06, 141.05, 140.95, 140.62, 139.16, 128.74, 128.51, 125.44, 115.90, 113.73, 69.68, 44.58, 43.76, 33.02. HRMS (ESI), found 362.1986 C₂₁H₂₃N₅O, [M+H]⁺, requires 362.1981.

### 1-(2′-(2-(pyridin-3-yl)ethoxy)-[1,1′-biphenyl]-3-yl)piperazine (12i)

Following general procedure D, 9 (100 mg, 0.36 mmol) was reacted with (3-(4-(tert-butoxycarbonyl)piperazin-1-yl)phenyl)boronic acid (137 mg, 0.44 mmol), the crude was purified by reverse phase chromatography (6 g C18 column; MeCN in water 0-100%). The resulting residue was reacted according to general procedure E. Afforded the title compound as a fluffy off-white solid (76 mg, 74%). ¹H NMR (CD₃OD, 400 MHz, δ, ppm) 8.63 (1H, d, J=5.7 Hz), 8.59 (1H, d, J=1.8 Hz), 8.31 (1H, dt, J=8.1, 1.6 Hz,), 7.85-7.80 (1H, m), 7.48 (1H, d, J=1.9 Hz), 7.44-7.40 (2H, m), 7.29 (1H, ddd, J=8.3, 7.4, 1.7 Hz), 7.22 (1H, dd, J=7.6, 1.7 Hz), 7.20-7.17 (1H, m), 7.06 (1H, dd, J=8.3, 0.9 Hz), 6.99 (1H, td, J=7.5, 1.0 Hz), 4.30 (2H, t, J=6.0 Hz), 3.78 (4H, dd, J=6.5, 4.0 Hz), 3.64 (4H, dd, J=6.5, 4.0 Hz,), 3.28 (2H, t, J=6.0 Hz). ¹³C NMR (CD₃OD, 100 MHz, δ, ppm) 156.46, 148.87, 146.35, 142.48, 141.98, 141.47, 140.54, 131.70, 131.15, 130.66, 130.62, 128.76, 128.04, 122.68, 121.57, 118.77, 114.09, 68.66, 50.65, 43.52, 33.38. HRMS (ESI), found 360.2077 C₂₃H₂₅N₃O, [M+H]⁺, requires 360.2076.

### 1-(4-(2-(2-(pyridin-3-yl)ethoxy)phenyl)pyridin-2-yl)piperazine (12j)

Following general procedure D, 9 (100 mg, 0.35 mmol) was reacted with 2-(4-tert-butoxycarbonylpiperazin-1-yl)pyridine-4-boronic acid, pinacol ester (174 mg, 0.45 mmol), the crude was purified by reverse phase chromatography (6 g C18 column; MeCN in water 0-100%). The resulting residue was reacted according to general procedure E. Afforded the title compound as a fluffy pale yellow solid (70 mg, 77%). ¹H NMR (CD₃OD, 400 MHz, δ, ppm) 8.76 (2H, s), 8.57 (1H, d, J=8.0 Hz), 8.03 (2H, d, J=6.6 Hz), 7.53-7.48 (2H, m), 7.44 (1H, d, J=1.2 Hz), 7.25-7.18 (2H, m), 7.13 (1H, td, J=7.6, 1.0 Hz), 4.44 (2H, t, J=6.1 Hz), 4.11-4.05 (4H, m), 3.54-3.49 (4H, m), 3.38 (2H, t, J=6.1 Hz,). ¹³C NMR (CD₃OD, 100 MHz, δ, ppm) 157.11, 156.86, 153.88, 148.73, 142.62, 140.77, 136.87, 133.45, 131.79, 128.46, 127.09, 122.96, 117.43, 114.08, 113.55, 68.80, 44.68, 43.73, 33.35. HRMS (ESI), found 361.2028 C₂₂H₂₄N₄O, [M+H]⁺, requires 361.2028.

### 1-(4′-fluoro-2′-(2-(pyridin-3-yl)ethoxy)-[1,1′-biphenyl]-3-yl)piperazine (12k)

Following general procedure D, 11 (100 mg, 0.33 mmol) was reacted with (3-(4-(tert-butoxycarbonyl)piperazin-1-yl)phenyl)boronic acid (129 mg, 0.42 mmol), the crude was purified by reverse phase chromatography (6 g C18 column; MeCN in water 0-100%). The resulting residue was reacted according to general procedure E. Afforded the title compound as a fluffy yellow solid (76 mg, 74%). ¹H NMR (CD₃OD, 400 MHz, δ, ppm) 8.69 (1H, d, J=5.6 Hz), 8.60 (1H, s), 8.28 (1H, dt, J=8.1, 1.6 Hz), 7.84 (1H, dd, J=8.0, 5.8 Hz), 7.35-7.28 (1H, m), 7.22 (1H, dd, J=8.5, 6.7 Hz), 7.07 (1H, ddd, J=8.3, 2.5, 0.8 Hz), 7.00-6.96 (1H, m), 6.90 (1H, dd, J=10.9, 2.5 Hz), 6.87-6.83 (1H, m), 6.76 (1H, td, J=8.4, 2.5 Hz), 4.33 (2H, t, J=5.8 Hz), 3.48 (4H, dd, J=6.8, 3.1 Hz), 3.42 (4H, dd, J=6.8, 3.0 Hz), 3.27 (2H, t, J=5.8 Hz). ¹³C NMR (CD₃OD, 100 MHz, δ, ppm) 164.41 (d, J=243.7 Hz), 157.67 (d, J=10 Hz), 157.62, 151.11, 148.94, 142.47, 141.29, 140.57, 140.32, 132.53 (d, J=10 Hz), 129.94, 128.62 (d, J=3.3 Hz), 127.97, 124.10, 119.47, 116.88, 108.62 (d, J=21.2 Hz), 101.86 (d, J=25.7 Hz), 68.87, 48.10, 44.73, 33.26. HRMS (ESI), found 378.1986 C₂₃H₂₄FN₃O, [M+H]⁺, requires 378.1982.

### 1-(4-(4-fluoro-2-(2-(pyridin-3-yl)ethoxy)phenyl)pyridin-2-yl)piperazine (12l)

Following general procedure D, 11 (90 mg, 0.30 mmol) was reacted with (3-(4-(tert-butoxycarbonyl)piperazin-1-yl)phenyl)boronic acid (117 mg, 0.38 mmol), the crude was purified by reverse phase chromatography (6 g C18 column; MeCN in water 0-100%). The resulting residue was reacted according to general procedure E. Afforded the title compound as a fluffy off-white solid (50 mg, 69%). ¹H NMR (CD₃OD, 400 MHz, δ, ppm) 8.76-8.72 (2H, m), 8.58-8.54 (1H, m), 8.06-7.99 (2H, m), 7.54 (1H, dd, J=8.6, 6.5 Hz), 7.44-7.41 (1H, m), 7.21 (1H, dd, J=6.6, 1.5 Hz), 7.03 (1H, dd, J=10.9, 2.4 Hz), 6.90-6.83 (1H, m), 4.42 (2H, t, J=6.1 Hz), 4.10-4.04 (4H, m), 3.53-3.46 (4H, m), 3.37 (2H, t, J=6.1 Hz,). ¹³C NMR (CD₃OD, 100 MHz, δ, ppm) 166.54 (d, J=248.6 Hz), 158.47 (d, J=10.5 Hz), 156.32, 153.70, 148.89, 142.65, 140.93, 140.82, 136.64, 133.44 (d, J=10.5 Hz), 128.49, 123.20 (d, J=3.3 Hz), 117.23, 113.66, 109.49 (d, J=21.9 Hz), 102.20 (d, J=26.5 Hz), 69.26, 44.71, 43.71, 33.11. HRMS (ESI), found 379.1925 C₂₂H₂₃FN₄O, [M+H]⁺, requires 379.1934.

### 1-(3-(3-(2-(3,5-dimethyl-1H-pyrazol-4-yl)ethoxy)pyridin-2-yl)phenyl)piperazine (12m)

Following general procedure D, 7 (35 mg, 0.12 mmol) was reacted with (3-(4-(tert-butoxycarbonyl)piperazin-1-yl)phenyl)boronic acid (45 mg, 0.15 mmol), the crude was purified by reverse phase chromatography (6 g C18 column; MeCN in water 0-100%). The resulting residue was reacted according to general procedure E. Afforded the title compound as a fluffy off-white solid (12 mg, 47%). ¹H NMR (CD₃OD, 400 MHz, δ, ppm) 8.34 (1H, d, J=5.6 Hz), 8.30 (1H, d, J=8.6 Hz), 7.94 (1H, dd, J=8.6, 5.7 Hz), 7.44 (1H, t, J=7.9 Hz), 7.33-7.27 (2H, m), 7.09 (1H, d, J=7.5 Hz), 4.36 (2H, t, J=5.8 Hz), 3.55-3.47 (4H, m), 3.39-3.32 (4H, m), 2.95 (2H, t, J=5.6 Hz), 2.09 (6H, s). ¹H NMR (CD₃OD, 400 MHz, δ, ppm) 156.38, 151.85, 145.77, 144.98, 134.02, 131.17, 131.07, 130.65, 128.31, 122.92, 120.63, 118.65, 116.20, 70.67, 47.32, 44.58, 22.80, 9.60. HRMS (ESI), found 378.2294 C₂₂H₂₇N₅O, [M+H]⁺, requires 378.2294.

### 1-(4-(2-(2-(3,5-dimethyl-1H-pyrazol-4-yl)ethoxy)phenyl)pyridin-2-yl)piperazine (12n)

Following general procedure D, 6 (90 mg, 0.30 mmol) was reacted with 2-(4-tert-butoxycarbonylpiperazin-1-yl)pyridine-4-boronic acid, pinacol ester (148 mg, 0.38 mmol), the crude was purified by reverse phase chromatography (6 g C18 column; MeCN in water 0-100%). The resulting residue was reacted according to general procedure E. Afforded the title compound as a fluffy off-white solid (44 mg, 79%). ¹H NMR (CD₃OD, 400 MHz, δ, ppm) 8.05 (1H, d, J=6.6 Hz), 7.55-7.49 (3H, m), 7.29 (1H, dd, J=6.6, 1.3 Hz), 7.21 (1H, d, J=8.0 Hz), 7.14 (1H, td, J=7.5, 0.8 Hz), 4.26 (2H, t, J=6.6 Hz), 4.15-4.08 (4H, m), 3.56-3.50 (4H, m), 3.00 (2H, t, J=6.6 Hz), 2.32 (6H, s). ¹³C NMR (CD₃OD, 100 MHz, δ, ppm) 157.49, 157.06, 153.72, 145.58, 136.77, 133.45, 131.86, 127.06, 122.79, 117.48, 116.44, 114.16, 113.63, 68.48, 49.00, 44.71, 43.73, 23.12, 9.79. HRMS (ESI), found 378.2292 C₂₂H₂₇N₅O, [M+H]⁺, requires 378.2294.

### 3-(2-(3,5-dimethyl-1H-pyrazol-4-yl)ethoxy-2′-(piperazin-1-yl)-2,4′-bypyridine (12o)

Following general procedure D, 7 (50 mg, 0.17 mmol) was reacted with 2-(4-tert-Butoxycarbonylpiperazin-1-yl)pyridine-4-boronic acid, pinacol ester (82 mg, 0.21 mmol), the crude was purified by reverse phase chromatography (6 g C18 column; MeCN in water 0-100%). The resulting residue was reacted according to general procedure E. Afforded the title compound as a fluffy off-white solid (25 mg, 80%). ¹H NMR (CD₃OD, 400 MHz, δ, ppm) 8.43 (1H, brs), 8.20 (1H, d, J=5.7 Hz), 7.93 (1H, d, J=8.3 Hz), 7.78 (1H, brs), 7.73 (1H, brs), 7.46 (1H, d, J=5.6 Hz), 4.37 (2H, t, J=5.6 Hz), 4.08 (4H, brs), 3.49 (4H, brs), 3.07 (2H, t, J=5.6 Hz), 2.32 (6H, s). ¹³C NMR (CD₃OD, 100 MHz, δ, ppm) 155.47, 150.83, 146.32, 145.85, 142.76, 140.73, 138.10, 128.69, 125.21, 115.98, 115.65, 114.51, 112.87, 69.38, 44.43, 43.91, 23.05, 9.87. HRMS (ESI), found 379.2247 C₂₁H₂₆N₆O, [M+H]⁺, requires 379.2246.

### 3-(2′-(3,5-dimethyl-1H-pyrazol-4-yl)ethoxy-4′-fluoro-[1,1′-biphenyl]-3-yl)piperazine (12p)

Following general procedure D, 8 (75 mg, 0.23 mmol) was reacted with (3-(4-(tert-butoxycarbonyl)piperazin-1-yl)phenyl)boronic acid (92 mg, 0.30 mmol), the crude was purified by reverse phase chromatography (6 g C18 column; MeCN in water 0-100%). The resulting residue was reacted according to general procedure E. Afforded the title compound as a fluffy pale yellow solid (12 mg, 47%). ¹H NMR (CD₃OD, 400 MHz, δ, ppm) 7.28 (1H, t, J=7.8 Hz), 7.19 (1H, dd, J=8.3, 6.9 Hz), 7.06 (1H, d, J=8.1 Hz), 7.01 (1H, brs), 6.88 (1H, dd, J=11.1, 2.2 Hz), 6.82 (1H, d, J=7.5 Hz), 6.74 (1H, td, J=8.3, 2.1 Hz), 4.13 (2H, t, J=5.3 Hz), 3.52-3.46 (4H, m), 3.45-3.40 (4H, m), 2.89 (2H, t, J=5.2 Hz), 2.11 (6H, s). ¹³C NMR (CD₃OD, 100 MHz, δ, ppm) 164.39 (d, J=243.3 Hz), 158.06 (d, J=9.9 Hz), 151.24, 145.64, 140.78, 132.72 (d, J=9.8 Hz), 130.06, 128.66 (d, J=3.3 Hz), 123.95, 119.50, 116.94, 116.87, 108.22 (d, J=21.2 Hz), 101.64 (d, J=25.9 Hz), 68.96, 48.13, 44.73, 23.11, 9.50. HRMS (ESI), found 395.2247 C₂₃H₂₇FN₄O, [M+H]⁺, requires 395.2247.

### 1-(4-(2-(2-(3,5-dimetyl-1H-pyrazol-4-yl)ethoxy)-4-fluorophenyl) piridin-2-yl)piperazine (12q)

Following general procedure D, 8 (30 mg, 0.10 mmol) was reacted with 2-(4-tert-Butoxycarbonylpiperazin-1-yl)pyridine-4-boronic acid, pinacol ester (47 mg, 0.12 mmol), the crude was purified by reverse phase chromatography (6 g C18 column; MeCN in water 0-100%). The resulting residue was reacted according to general procedure E. Afforded the title compound as a fluffy off-white solid (44 mg, 79%). ¹H NMR (CD₃OD, 400 MHz, δ, ppm) 8.05 (1H, d, J=6.6 Hz), 7.58 (1H, dd, J=8.5, 6.6 Hz), 7.53 (1H, s), 7.28 (1H, dd, J=6.6, 1.1 Hz), 7.05 (1H, dd, J=11.0, 2.3 Hz), 6.90 (1H, td, J=8.3, 2.3 Hz), 4.25 (2H, t, J=6.6 Hz), 4.14-4.08 (4H, m), 3.56-3.49 (4H, m), 3.01 (2H, t, J=6.6 Hz), 2.32 (6H, s). ¹³C NMR (CD₃OD, 100 MHz, δ, ppm) 166.51 (d, J=248.3 Hz), 158.70 (d, J=10.5 Hz), 156.51, 153.71, 145.63, 136.84, 133.48 (d, J=10.5 Hz), 129.93, 123.22 (d, J=3.3 Hz), 117.32, 116.22, 113.64, 109.31 (d, J=22.0 Hz), 102.23 (d, J=26.4 Hz), 68.96, 44.72, 43.72, 22.94, 9.79. HRMS (ESI), found 396.2201 C₂₂H₂₆FN₅O, [M+H]⁺, requires 396.2200.

### tert-butyl 4-(6-chloropyrimidin-4-yl)piperazine-1-carboxylate (13)

To a solution of 4,6-dichloro-pyrimidine (500 mg, 3.35 mmol) was dissolved in THF (10 mL) in a microwave vial (10-20 mL) and treated with tert-butyl piperazine-1-carboxylate (688 mg, 3.69 mmol) and DIPEA (0.87 mL, 5.03 mmol). The reaction mixture was heated at 145° C. under microwave irradiation for 30 minutes. The resulting mixture was concentrated in a vacuum, and the residue was partitioned between DCM (50 mL) and water (50 mL). The organic fraction was washed with brine (50 mL), and the organic phase was dried over Na₂SO₄, concentrated under reduced pressure to yield the title compound (920 mg, 92%) as a light brown solid. The intermediary 13 was used without any purification in the following reactions. ¹H NMR (CDCl₃, 400 MHz, δ, ppm) 1H NMR (DMSO-d₆, 400 MHz, δ, ppm) 8.32 (1H, s), 6.93 (1H, s), 3.62 (4H, brs), 3.39-3.33 (4H, m), 1.38 (9H, s).

### tert-butyl 4-(6-(2-hydroxyphenyl)pyrimidin-4-yl)piperazine-1-carboxylate (14)

Following general procedure D, 13 (200 mg, 0.67 mmol) was reacted with 2-hydroxybenzeneboronic acid (115 mg, 0.83 mmol). The crude was purified by flash column chromatography by elution with Pentane/EtOAc in gradient to afford 130 mg (54%) of the title compound as a beige solid. ¹H NMR (CDCl₃, 400 MHz, δ, ppm) 8.49 (1H, d, J=1.0 Hz), 7.65 (1H, dd, J=8.1, 1.5 Hz), 7.28 (1H, td, J=7.9, 7.2, 1.6 Hz,), 6.94 (1H, dd, J=8.3, 1.1 Hz,), 6.85-6.80 (2H, m), 3.68 (4H, d, J=5.0 Hz), 3.55-3.50 (4H, m,), 1.47 (9H, s).

### tert-butyl 4-(6-(4-fluoro-2-hydroxyphenyl)pyrimidin-4-yl)piperazine-1-carboxylate (15)

Following general procedure D, 13 (90 mg, 0.25 mmol) was reacted with 4-fluoro-2-hydroxybenzene boronic acid (75 mg, 0.48 mmol). The crude was purified by flash column chromatography by elution with Pentane/EtOAc in gradient to afford 91 mg (63%) of the title compound as a beige solid. ¹H NMR (CDCl₃, 400 MHz, δ, ppm) 8.49 (1H, d, J=1.0 Hz), 7.64 (1H, dd, J=9.0, 7.4 Hz), 6.75 (1H, d, J=1.0 Hz), 6.64 (1H, dd, J=9.1, 2.6 Hz), 6.55 (1H, ddd, J=9.0, 7.4, 2.6 Hz), 3.75-3.69 (4H, m), 3.58-3.52 (4H, m), 1.48 (9H, s).

### 4-(piperazin-1-yl)-6-(2-(2-(1,3,5-trimethyl-1H-pyrazol-4-yl)ethoxy)phenyl)pyrimidine (16a)

Following general procedure C, 14 (70 mg, 0.19 mmol) was reacted with 1 (33 mg, 0.23 mmol) the crude was purified by reverse phase chromatography (6 g C18 column; MeCN in water 0-100%). The resulting residue was reacted according to general procedure E. Afforded the title compound as a fluffy yellow solid (8 mg 38%) ¹H NMR (CD₃OD, 400 MHz, δ, ppm) 8.84 (1H, brs), 7.70-7.56 (2H, m), 7.38 (1H, brs), 7.24 (1H, d, J=8.3 Hz), 7.19 (1H, t, J=7.1 Hz), 4.45 (2H, brs), 4.24 (4H, brs), 3.91 (3H, s), 3.47 (4H, brs), 3.02 (2H, brs), 2.33 (3H, s), 2.29 (3H, s). ¹³C NMR (CD₃OD, 100 MHz, δ, ppm) 162.13, 155.89, 152.83, 150.44, 144.96, 142.95, 133.93, 130.88, 121.45, 119.13, 115.31, 112.59, 102.97, 67.33, 42.74, 40.91, 34.33, 21.94, 8.27, 8.19. HRMS (ESI), found 393.2403 C₂₂H₂₈N₆O, [M+H]⁺, requires 393.2403.

### 4-(4-fluoro-2-(2-(1,3,5-trimethyl-1H-pyrazol-4-yl)ethoxy)phenyl)-6-(piperazin-1-yl)pyrimidine (16b)

Following general procedure C, 15 (70 mg, 0.18 mmol) was reacted with 1 (33 mg, 0.23 mmol) the crude was purified by reverse phase chromatography (6 g C18 column; MeCN in water 0-100%). The resulting residue was reacted according to general procedure E. Afforded the title compound as a fluffy brownish white solid (8 mg 39%) ¹H NMR (CD₃OD, 400 MHz, δ, ppm) 8.86 (1H, brs), 7.77-7.68 (1H, m), 7.39 (1H, brs), 7.10 (1H, d, J=10.5 Hz), 6.97 (1H, t, J=6.9 Hz), 4.49-4.34 (2H, m), 4.23 (4H, brs), 3.92 (3H, s), 3.49 (4H, brs), 3.04 (2H, brs), 2.34 (3H, s), 2.30 (3H, s). ¹³C NMR (CD₃OD, 100 MHz, δ, ppm) 167.57 (d, J=205.7 Hz), 163.50, 159.12 (d, J=10.9 Hz), 153.31, 151.91, 146.39, 144.38, 134.22 (d, J=10.9 Hz), 116.92 (d, J=2.9 Hz), 116.49, 109.63 (d, J=22.4 Hz), 104.55, 102.35 (d, J=26.8 Hz), 69.38, 44.18, 42.34, 35.81, 23.20, 9.74, 9.68. HRMS (ESI), found 411.2309 C₂₂H₂₇FN₆₀, [M+H]⁺, requires 411.2309.

### 4-(piperazin-1-yl)-6-(2-(2-(pyridine-3-yl)ethoxy)phenyl)pyrimidine (16c)

Following general procedure C, 14 (66 mg, 0.18 mmol) was reacted with 3-(2-hydroxyethyl)pyridine (28 mg, 0.23 mmol) the crude was purified by reverse phase chromatography (6 g C18 column; MeCN in water 0-100%). The resulting residue was reacted according to general procedure E. Afforded the title compound as a fluffy brownish white solid (22 mg 45%) ¹H NMR (DMSO-d₆, 400 MHz, δ, ppm) 10.16 (1H, brs), 10.06 (1H, brs), 8.89 (1H, s), 8.82 (1H, s), 8.79 (1H, d, J=5.0 Hz), 8.46 (1H, d, J=7.8 Hz), 7.95 (1H, brt, J=6.6 Hz), 7.61-7.58 (2H, m), 7.34 (1H, s), 7.28 (1H, d, J=8.5 Hz), 7.16 (1H, t, J=7.4 Hz), 4.41 (2H, t, J=5.8 Hz), 4.19 (4H, brs), 3.34-3.23 (6H, m). ¹³C NMR (DMSO-d₆, 100 MHz, δ, ppm) 161.35, 155.64, 152.03, 151.26, 145.70, 142.05, 140.15, 138.35, 133.48, 131.05, 126.56, 121.19, 119.93, 113.01, 103.36, 67.83, 48.53, 41.89, 31.43. HRMS (ESI), found 362.2000 C₂₁H₂₃N₅O, [M+H]⁺, requires 362.1981.

### 4-(4-fluoro-2-(2-(pyridine-3-yl)ethoxy)phenyl)-6-(piperazin-1-yl)pyrimidine (16d)

Following general procedure C, 15 (88 mg, 0.23 mmol) was reacted with 3-(2-hydroxyethyl)pyridine (36 mg, 0.29 mmol) the crude was purified by reverse phase chromatography (6 g C18 column; MeCN in water 0-100%). The resulting residue was reacted according to general procedure E. Afforded the title compound as a fluffy brownish white solid (8 mg 39%) ¹H NMR (DMSO-d₆, 400 MHz, δ, ppm) 9.88 (1H, brs), 8.87 (1H, brs), 8.78 (2H, d, J=8.6 Hz), 8.42 (1H, d, J=7.6 Hz), 7.97-7.90 (1H, m), 7.66 (1H, dd, J=8.5, 6.8 Hz), 7.32 (1H, brs), 7.25 (1H, dd, J=11.3, 2.1 Hz), 7.05 (1H, td, J=8.4, 2.1 Hz), 4.43 (2H, t, J=6.3 Hz), 4.16 (4H, brs), 3.31-3.26 (6H, m). ¹³C NMR (DMSO-d₆, 100 MHz, δ, ppm) 165.02 (d, J=248.0 Hz), 161.33, 157.43 (d, J=11.0 Hz), 151.74, 145.36, 142.41, 140.55, 137.96, 132.83 (d, J=11.0 Hz), 126.46, 116.93, 108.02 (d, J=22.1 Hz), 103.41, 101.43 (d, J=25.5 Hz), 68.40, 42.03, 31.24. HRMS (ESI), found 380.1888 C₂₁H₂₂FN₅O, [M+H]⁺, requires 380.1887.

### 4-(2-(2-(3,5-dimethyl-1H-pyrazol-4-yl)ethoxy)-4-fluorophenyl)-6-(piperazin-1-yl)pyrimidine (16e)

Following general procedure C, 15 (100 mg, 0.26 mmol) was reacted with 2 (46 mg, 0.32 mmol) the crude was purified by reverse phase chromatography (6 g C18 column; MeCN in water 0-100%). The resulting residue was reacted according to general procedure E. Afforded the title as a fluffy brownish white solid (15 mg 39%) ¹H NMR (CD₃OD, 400 MHz, δ, ppm) 8.85 (1H, s), 7.74 (1H, dd, J=8.5, 6.4 Hz), 7.40 (1H, s), 7.12 (1H, dd, J=8.5, 2.1 Hz), 6.96 (1H, td, J=8.4, 2.0 Hz), 4.47 (2H brs), 4.31-4.17 (4H, m), 3.50 (4H, brs), 3.06 (2H, t, J=6.8 Hz), 2.35 (6H, s). ¹³C NMR (CD₃OD, 100 MHz, δ, ppm) 167.61 (d, J=250.9 Hz), 163.50, 159.14 (d, J=11.0 Hz), 153.37, 151.87, 145.66, 134.15 (d, J=11.0 Hz), 116.98 (d, J=3.1 Hz), 115.94, 109.66 (d, J=22.4 Hz), 104.47, 102.38 (d, J=26.8 Hz), 69.22, 44.04, 22.80, 9.77. HRMS (ESI), found 397.2154 C₂₁H₂₅FN₆₀, [M+H]⁺, requires 397.2152.

### 4-(2-(2-(3,5-dimethyl-1H-pyrazol-4-yl)ethoxy)phenyl)-6-(piperazin-1-yl)pyrimidine (16f)

Following general procedure C, 14 (70 mg, 0.19 mmol) was reacted with 2 (34 mg, 0.24 mmol) the crude was purified by reverse phase chromatography (6 g C18 column; MeCN in water 0-100%). The resulting residue was reacted according to general procedure E. Afforded the title compound as fluffy brownish white solid (41 mg, 72%) ¹H NMR (CD₃OD, 400 MHz, δ, ppm) 8.85 (1H, brs), 7.69 (1H, dd, J=7.6, 1.4 Hz), 7.66-7.61 (1H, m), 7.40 (1H, brs), 7.28 (1H, d, J=8.4 Hz), 7.20 (1H, t, J=7.5 Hz), 4.48 (2H, brs), 4.29 (2H, t, J=6.8 Hz), 4.22 (2H, brs), 3.50 (4H, brs), 3.05 (2H, t, J=6.8 Hz), 2.35 (6H, s). ¹³C NMR (CD₃OD, 100 MHz, δ, ppm) 163.52, 157.30, 154.24, 151.79, 145.61, 135.34, 132.22, 122.87, 120.55, 116.18, 114.04, 104.35, 68.59, 43.97, 42.26, 22.93, 9.75. HRMS (ESI), found 379.2249 C₂₁H₂₆N₆O, [M+H]⁺, requires 379.2246.

### 3-(3-(2-(1,3,5-trimethyl-1H-pyrazol-4-yl)ethoxy)pyridin-2-yl)phenol (17)

Following general procedure D, 4 (100 mg, 0.32 mmol) was reacted with 3-hydroxyphenylboronic acid (56 mg, 0.39 mmol), the crude was purified by flash column chromatography by elution with Pentane/EtOAc in gradient to afford 89 mg (74%) of the title compound as a beige solid. ¹H NMR (CDCl₃, 400 MHz, δ, ppm) 8.79 (1H, brs), 8.24 (1H, dd, J=4.6, 1.4 Hz), 7.29 (1H, dt, J=7.7, 1.3 Hz), 7.24-7.18 (2H, m), 7.16 (1H, dd, J=8.3, 4.6 Hz), 6.85-6.80 (2H, m), 4.01 (2H, t, J=6.6 Hz), 3.68 (3H, s), 2.81 (2H, t, J=6.5 Hz), 2.10 (3H, s), 2.03 (3H, s).

### 2′-(2-(1,3,5-trimethyl-1H-pyrazol-4-yl)ethoxy)-[1,1′-biphenyl]-3-ol (18)

Following general procedure D, 3 (100 mg, 0.32 mmol) was reacted with 3-hydroxyphenylboronic acid (56 mg, 0.39 mmol), the crude was purified by flash column chromatography by elution with Pentane/EtOAc in gradient to afford 80 mg (76%) of the title compound as a beige solid. ¹H NMR (CDCl₃, 400 MHz, δ, ppm) 7.28-7.22 (4H, m), 7.00-6.95 (1H, m), 6.94-6.89 (2H, m), 6.84 (1H, ddd, J=8.1, 2.5, 1.0 Hz), 6.08 (1H, dd, J=2.3, 1.6 Hz), 4.09 (2H, t, J=6.2 Hz), 3.72 (3H, s), 2.82 (2H, t, J=6.2 Hz), 2.08 (3H, s), 2.04 (3H, S).

### 4′-fluoro-2′-(2-(1,3,5-trimethyl-1H-pyrazol-4-yl)ethoxy)-[1,1′-biphenyl]-3-ol (19)

Following general procedure D, 5 (90 mg, 0.27 mmol) was reacted with 3-hydroxyphenylboronic acid (48 mg, 0.34 mmol), and the crude was purified by flash column chromatography by elution with Pentane/EtOAc in gradient to afford 73 mg (78%) of the title compound as a beige solid. ¹H NMR (CDCl₃, 400 MHz, δ, ppm) 7.25-7.17 (2H, m), 6.87-6.82 (2H, m), 6.70-6.62 (2H, m), 6.10-6.08 (1H, m), 4.04 (2H, t, J=6.2 Hz), 3.72 (3H, s), 2.82 (2H, t, J=6.2 Hz), 2.09 (3H, s), 2.02 (3H, s).

### 3-(3-(2-(pyridin-3-yl)ethoxy)pyridin-2-yl)phenol (20)

Following general procedure D, 10 (100 mg, 0.35 mmol) was reacted with 3-hydroxyphenylboronic acid (61 mg, 0.44 mmol), and the crude was purified by flash column chromatography by elution with Pentane/EtOAc in gradient to afford 75 mg (71%) of the title compound as a beige solid. ¹H NMR (CDCl₃, 400 MHz, δ, ppm) 8.57 (1H, d, J=1.7 Hz), 8.47 (1H, dd, J=4.9, 1.6 Hz), 8.23 (1H, dd, J=3.8, 2.2 Hz), 7.60-7.55 (1H, m), 7.30-7.26 (1H, m), 7.23 (2H, dt, J=7.7, 1.4 Hz), 7.16-7.13 (2H, m), 6.99 (1H, dd, J=2.5, 1.5 Hz), 6.92 (1H, ddd, J=7.8, 2.5, 1.4 Hz), 4.10 (2H, t, J=5.8 Hz), 3.02 (2H, t, J=5.7 Hz).

### 2′-(2-(pyridin-3-yl)ethoxy)-[1,1′-biphenyl]-3-ol (21)

Following general procedure D, 9 (100 mg, 0.36 mmol) was reacted with 3-hydroxyphenylboronic acid (62 mg, 0.45 mmol), the crude was purified by flash column chromatography by elution with Pentane/EtOAc in gradient to afford 78 mg (74%) of the title compound as a beige solid. ¹H NMR (CDCl₃, 400 MHz, δ, ppm) 10.03 (1H, brs), 8.53 (1H, s), 8.45 (1H, d, J=3.9 Hz), 7.54 (1H, d, J=7.8 Hz), 7.34-7.20 (4H, m), 7.02-6.95 (2H, m), 6.89 (3H, m), 4.08 (2H, t, J=5.7 Hz), 2.96 (2H, t, J=5.6 Hz).

### 4′-fluoro-2′-(2-(pyridin-3-yl)ethoxy)-[1,1′-biphenyl]-3-ol (22)

Following general procedure D, 11 (100 mg, 0.33 mmol) was reacted with 3-hydroxyphenylboronic acid (58 mg, 0.42 mmol), and the crude was purified by flash column chromatography by elution with Pentane/EtOAc in gradient to afford 90 mg (86%) of the title compound as a beige solid. ¹H NMR (CDCl₃, 400 MHz, δ, ppm) 9.82 (1H, s), 8.53 (1H, d, J=2.0 Hz), 8.46 (1H, dd, J=4.9, 1.6 Hz), 7.57 (1H, dt, J=7.8, 1.9 Hz), 7.30-7.18 (3H, m), 6.93 (1H, ddd, J=8.2, 2.5, 0.9 Hz), 6.82 (1H, dt, J=7.6, 1.2 Hz), 6.71 (1H, dd, J=2.4, 1.7 Hz), 6.66 (1H, td, J=8.2, 2.4 Hz), 6.57 (1H, dd, J=11.0, 2.4 Hz), 4.05 (2H, t, J=5.6 Hz), 2.99 (2H, t, J=5.6 Hz).

### 3-(3-(2-(3,5-dimethyl-1H-pyrazol-4-yl)ethoxy)pyridin-2-yl)phenol (23)

Following general procedure D, 7 (55 mg, 0.18 mmol) was reacted with 3-hydroxyphenylboronic acid (32 mg, 0.23 mmol), and the crude was purified by flash column chromatography by elution with Pentane/EtOAc in gradient to afford 40 mg (70%) of the title compound as a beige solid. ¹H NMR (CDCl₃, 400 MHz, δ, ppm) 8.21 (1H, d, J=5.8 Hz), 7.22-7.11 (4H, m), 6.81-6.77 (2H, m), 3.98 (2H, t, J=5.4 Hz), 2.75 (2H, t, J=5.4 Hz), 2.00 (6H, s).

### 2′-(2-(3,5-dimethyl-1H-pyrazol-4-yl)ethoxy)-[1,1′-biphenyl]-3-ol (24)

Following general procedure D, 6 (51 mg, 0.17 mmol) was reacted with 3-hydroxyphenylboronic acid (36 mg, 0.26 mmol), and the crude was purified by flash column chromatography by elution with Pentane/EtOAc in gradient to afford 43 mg (80%) of the title compound as a beige solid. ¹H NMR (CDCl₃, 400 MHz, δ, ppm) 7.26-7.23 (1H, m), 7.22-7.16 (2H, m), 6.94 (1H, td, J=7.5, 1.0 Hz), 6.90 (1H, d, J=8.3 Hz), 6.86 (1H, dt, J=7.6, 1.2 Hz), 6.84-6.79 (1H, m), 6.49-6.46 (1H, m), 4.00 (2H, t, J=6.2 Hz), 2.72 (2H, t, J=6.2 Hz), 1.99 (6H, s).

### 2′-(2-(3,5-dimethyl-1H-pyrazol-4-yl)ethoxy)-4′-fluoro-[1,1′-biphenyl]-3-ol (25)

Following general procedure D, 8 (100 mg, 0.31 mmol) was reacted with 3-hydroxyphenylboronic acid (55 mg, 0.40 mmol), and the crude was purified by flash column chromatography by elution with Pentane/EtOAc in gradient to afford 71 mg (68%) of the title compound as a beige solid. ¹H NMR (CDCl₃, 400 MHz, δ, ppm) 7.21-7.16 (1H, m), 7.13 (1H, dd, J=8.3, 6.9 Hz), 6.82 (2H, dd, J=8.3, 4.2 Hz), 6.68-6.60 (2H, m), 6.50-6.46 (1H, m), 3.98 (2H, t, J=6.1 Hz), 2.73 (2H, t, J=6.1 Hz), 1.99 (6H, s).

### N,N-dimethyl-2-(3-(3-(2-(1,3,5-trimethyl-1H-pyrazol-4-yl)ethoxy)pyridin-2-yl)phenoxy)-ethan-1-amine (26a)

Following general procedure C, 17 (100 mg, 0.31 mmol) was reacted with 2-(dimethylamino)ethanol (34 mg, 0.38 mmol). The crude was purified by reverse phase chromatography (6 g C18 column; MeCN in water 0-100%) to afford 33 mg (41%) of the title compound as a brown resin. ¹H NMR (CD₃OD, 400 MHz, δ, ppm) 8.14 (1H, dd, J=4.7, 1.3 Hz), 7.51 (1H, dd, J=8.4, 1.3 Hz), 7.35-7.29 (2H, m), 7.25 (1H, dd, J=2.5, 1.5 Hz), 7.21 (1H, dt, J=7.6, 1.1 Hz), 7.01 (1H, ddd, J=8.2, 2.5, 1.0 Hz), 4.15 (2H, t, J=5.5 Hz), 4.09 (2H, t, J=6.5 Hz), 3.61 (3H, s), 2.89 (2H, t, J=5.5 Hz), 2.79 (2H, t, J=6.5 Hz), 2.42 (6H, s), 2.02 (3H, s), 1.97 (3H, s). ¹³C NMR (CD₃OD, 100 MHz, δ, ppm) 159.68, 154.52, 149.60, 146.89, 141.57, 140.12, 139.22, 130.07, 124.94, 123.27, 122.12, 116.46, 115.85, 113.38, 69.82, 66.27, 58.95, 45.63, 35.73, 24.44, 11.60, 9.38. HRMS (ESI), found 395.2446 C₂₃H₃₀N₄O₂, [M+H]⁺, requires 395.2447.

### N,N-dimethyl-2-((2′-(2-(1,3,5-trimethyl-1H-pyrazol-4-yl)ethoxy)-[1,1′-biphenyl]-3-yl)oxy)-ethan-1-amine (26b)

Following general procedure C, 18 (100 mg, 0.31 mmol) was reacted with 2-(dimethylamino)ethanol (34 mg, 0.38 mmol). The crude was purified by reverse phase chromatography (6 g C18 column; MeCN in water 0-100%) to afford 36 mg (45%) of the title compound as a brown resin. ¹H NMR (CD₃OD, 400 MHz, δ, ppm) 7.27-7.19 (3H, m), 7.01-6.95 (3H, m), 6.93 (1H, ddd, J=7.6, 1.5, 0.9 Hz), 6.89 (1H, ddd, J=8.3, 2.6, 0.9 Hz), 4.08 (2H, t, J=5.5 Hz), 3.96 (2H, t, J=6.5 Hz), 3.58 (3H, s), 2.82 (2H, t, J=5.5 Hz), 2.70 (2H, t, J=6.5 Hz), 2.37 (6H, s), 1.98 (3H, s), 1.91 (3H, s). ¹³C NMR (CD₃OD, 100 MHz, δ, ppm) 159.67, 157.13, 146.84, 141.77, 139.18, 132.35, 131.78, 129.97, 129.79, 123.35, 122.04, 116.86, 114.10, 113.98, 113.72, 69.71, 66.36, 59.06, 45.74, 35.69, 24.64, 11.56, 9.31. HRMS (ESI), found 394.2496 C₂₄H₃₁N₃O₂, [M+H]′, requires 394.2495.

### 2-((4′-fluoro-2′-(2-(1,3,5-trimethyl-1H-pyrazol-4-yl)ethoxy)-[1,1′-biphenyl]-3-yl)oxy)-N,N-dimethylethan-1-amine (26c)

Following general procedure C, 19 (100 mg, 0.29 mmol) was reacted with 2-(dimethylamino)ethanol (32 mg, 0.36 mmol). The crude was purified by reverse phase chromatography (6 g C18 column; MeCN in water 0-100%) to afford 60 mg (44%) of the title compound as a brown resin. ¹H NMR (CD₃OD, 400 MHz, δ, ppm) 7.24 (1H, t, J=7.9 Hz), 7.18 (1H, dd, J=8.4, 6.9 Hz), 6.95-6.92 (1H, m), 6.89 (2H, dt, J=7.5, 2.3 Hz), 6.78 (1H, dd, J=11.2, 2.4 Hz), 6.68 (1H, td, J=8.3, 2.5 Hz), 4.09 (2H, t, J=5.4 Hz), 3.97 (2H, t, J=6.4 Hz), 3.58 (3H, s), 2.85 (2H, t, J=5.4 Hz), 2.71 (2H, t, J=6.4 Hz), 2.40 (6H, s), 1.97 (3H, s), 1.90 (3H, s). ¹³C NMR (CD₃OD, 100 MHz, δ, ppm) 164.35 (d, J=243.1 Hz), 159.67, 158.31 (d, J=9.9 Hz), 146.85, 140.89, 139.21, 132.62 (d, J=9.9 Hz), 130.07, 128.32 (d, J=3.3 Hz), 123.38, 116.89, 114.06, 113.56, 107.94 (d, J=21.2 Hz), 101.57 (d, J=25.7 Hz), 69.91, 66.24, 58.99, 45.67, 39.46, 35.70, 24.41, 11.55, 9.30. HRMS (ESI), found 412.2401 C₂₄H₃₀FN₃O₂, [M+H]⁺, requires 412.2400.

### N,N-dimethyl-2-(3-(3-(2-(pyridin-3-yl)ethoxy)pyridin-2-yl)phenoxy)ethan-1-amine (26d)

Following general procedure C, 20 (100 mg, 0.34 mmol) was reacted with 2-(dimethylamino)ethanol (37 mg, 0.42 mmol). The crude was purified by reverse phase chromatography (6 g C18 column; MeCN in water 0-100%) to afford 30 mg (28%) of the title compound as a brown resin. ¹H NMR (CD₃OD, 400 MHz, δ, ppm) 8.39 (1H, s), 8.36 (1H, d, J=4.2 Hz), 8.16 (1H, dd, J=4.8, 1.3 Hz), 7.67-7.63 (1H, m), 7.54 (1H, dd, J=8.4, 1.3 Hz), 7.36-7.26 (3H, m), 7.24 (1H, dd, J=2.5, 1.5 Hz), 7.15 (1H, ddd, J=7.7, 1.5, 1.0 Hz), 7.01 (1H, ddd, J=8.2, 2.6, 1.0 Hz), 4.31 (2H, t, J=6.1 Hz), 4.13 (2H, t, J=5.5 Hz), 3.10 (2H, t, J=6.0 Hz), 2.81 (2H, t, J=5.5 Hz), 2.36 (6H, s). ¹³C NMR (CD₃OD, 100 MHz, δ, ppm) 158.37, 152.90, 149.17, 148.02, 146.65, 140.30, 138.33, 137.60, 135.01, 128.50, 123.59, 123.53, 121.86, 120.46, 115.15, 114.39, 68.51, 65.20, 57.67, 44.38, 32.18. HRMS (ESI), found 364.2026 C₂₂H₂₅N₃O₂, [M+H]⁺, requires 364.2025.

### N,N-dimethyl-2-((2′-(2-(pyridin-3-yl)ethoxy)-[1,1′-biphenyl]-3-yl)oxy)ethan-1-amine (26e)

Following general procedure C, 21 (95 mg, 0.32 mmol) was reacted with 2-(dimethylamino)ethanol (35 mg, 0.40 mmol). The crude was purified by reverse phase chromatography (6 g C18 column; MeCN in water 0-100%) to afford 50 mg (46%) of the title compound as a brown resin. ¹H NMR (CD₃OD, 400 MHz, δ, ppm) 8.40 (1H, dd, J=4.9, 1.4 Hz), 8.39-8.37 (1H, m), 7.70-7.66 (1H, m), 7.34-7.24 (4H, m), 7.04-7.02 (1H, m), 7.01-6.94 (4H, m), 4.29-4.26 (2H, m), 4.22 (2H, t, J=5.8 Hz), 3.36-3.32 (2H, m), 3.02 (2H, t, J=5.8 Hz), 2.78 (3H, s), 2.71 (3H, s). ¹³C NMR (CD₃OD, 100 MHz, δ, ppm) 158.95, 156.85, 150.77, 147.91, 141.40, 139.08, 137.16, 131.58, 131.56, 130.08, 129.95, 125.15, 123.85, 122.08, 117.74, 113.38, 113.18, 69.68, 64.19, 58.16, 44.44, 39.45, 33.82. HRMS (ESI), found 363.2073 C₂₃H₂₆N₂O₂, [M+H]⁺, requires 363.2073.

### 2-((4′-fluoro-2′-(2-(pyridin-3-yl)ethoxy)-[1,1′-biphenyl]-3-yl)oxy)-N,N-dimethylethan-1-amine (26f)

Following general procedure C, 22 (110 mg, 0.35 mmol) was reacted with 2-(dimethylamino)ethanol (40 mg, 0.44 mmol). The crude was purified by reverse phase chromatography (6 g C18 column; MeCN in water 0-100%) to afford 54 mg (40%) of the title compound as a brown resin. ¹H NMR (CD₃OD, 400 MHz, δ, ppm) 8.26 (2H, brs), 7.49 (1H, dt, J=7.9, 1.9 Hz), 7.21-7.13 (3H, m), 6.88-6.83 (2H, m), 6.80-6.77 (1H, m), 6.75 (1H, dd, J=11.1, 2.5 Hz), 6.64 (1H, td, J=8.3, 2.5 Hz), 4.11 (2H, t, J=6.0 Hz), 4.02 (2H, t, J=5.5 Hz), 2.94 (2H, t, J=5.9 Hz), 2.73 (2H, t, J=5.5 Hz), 2.29 (6H, s). ¹³C NMR (CD₃OD, 100 MHz, δ, ppm) 164.35 (d, J=243.3 Hz), 159.73, 158.02 (d, J=9.9 Hz), 150.54, 147.93, 140.46, 139.09, 136.58, 132.48 (d, J=9.9 Hz), 129.95, 128.09 (d, J=3.3 Hz), 124.94, 123.47, 117.03, 113.88, 108.10 (d, J=21.2 Hz), 101.33 (d, J=25.8 Hz), 69.83, 66.39, 59.05, 45.76, 33.56. HRMS (ESI), found 381.1978 C₂₃H₂₅FN₂₀₂, [M+H]⁺, requires 381.1978.

### 2-(3-(3-(2-(3,5-dimethyl-1H-pyrazol-4-yl)ethoxy)pyridin-2-yl)phenoxy)-N,N-dimethylethan-1-amine (26g)

Following general procedure C, 23 (40 mg, 0.13 mmol) was reacted with 2-(dimethylamino)ethanol (14 mg, 0.16 mmol). The crude was purified by reverse phase chromatography (6 g C18 column; MeCN in water 0-100%) to afford 22 mg (44%) of the title compound as a brown resin. ¹H NMR (CD₃OD, 400 MHz, δ, ppm) 8.14 (1H, d, J=4.7 Hz), 7.53 (1H, d, J=8.4 Hz), 7.36-7.30 (2H, m), 7.18 (1H, d, J=8.2 Hz), 7.16-7.14 (1H, m), 6.99 (1H, dd, J=8.2, 2.6 Hz), 4.14-4.10 (4H, m), 2.84-2.79 (4H, m), 2.37 (6H, s), 2.03 (6H, s). ¹³C NMR (CD₃OD, 100 MHz, δ, ppm) 158.28, 153.11, 148.11, 140.00, 138.57, 128.67, 123.49, 121.63, 120.47, 115.20, 113.99, 110.93, 68.19, 64.98, 57.72, 44.32, 22.53. HRMS (ESI), found 381.2290 C₂₂H₂₈N₄O₂, [M+H]⁺, requires 381.2291.

### 2-((2′-(2-(3,5-dimethyl-1H-pyrazol-4-yl)ethoxy)-[1,1′-biphenyl]-3-yl)oxy)-N,N-dimethylethan-1-amine (26h)

Following general procedure C, 24 (42 mg, 0.13 mmol) was reacted with 2-(dimethylamino)ethanol (15 mg, 0.17 mmol). The crude was purified by reverse phase chromatography (6 g C18 column; MeCN in water 0-100%) to afford 24 mg (46%) of the title compound as a brown resin. ¹H NMR (CD₃OD, 400 MHz, δ, ppm) 7.28-7.20 (3H, m), 7.03-6.99 (1H, m), 6.98 (1H, dd, J=7.4, 1.1 Hz), 6.95-6.91 (2H, m), 6.88 (1H, ddd, J=8.2, 2.4, 1.1 Hz), 4.07 (2H, t, J=5.4 Hz), 4.00 (2H, t, J=6.5 Hz), 2.79 (2H, t, J=5.4 Hz), 2.75 (2H, t, J=6.5 Hz), 2.36 (6H, s), 2.00 (6H, s). ¹³C NMR (CD₃OD, 100 MHz, δ, ppm) 159.66, 157.14, 141.69, 132.22, 131.82, 130.00, 129.77, 123.18, 121.93, 117.02, 113.85, 113.63, 112.71, 69.47, 66.33, 59.20, 45.79, 24.16. HRMS (ESI), found 380.2338 C₂₃H₂₉N₃O₂, [M+H]⁺, requires 380.2338.

### 2-((2′-(2-(3,5-dimethyl-1H-pyrazol-4-yl)ethoxy)-4′-fluoro-[1,1′-biphenyl]-3-yl)oxy)-N,N-dimethylethan-1-amine (26i)

Following general procedure C, 25 (90 mg, 0.27 mmol) was reacted with 2-(dimethylamino)ethanol (30 mg, 0.34 mmol). The crude was purified by reverse phase chromatography (6 g C18 column; MeCN in water 0-100%) to afford 40 mg (36%) of the title compound as a brown resin. ¹H NMR (CD₃OD, 400 MHz, δ, ppm) 7.26-7.22 (1H, m), 7.18 (1H, dd, J=8.5, 6.8 Hz), 6.88-6.84 (3H, m), 6.79 (1H, dd, J=11.2, 2.5 Hz), 6.67 (1H, td, J=8.3, 2.5 Hz), 4.05 (2H, t, J=5.4 Hz), 4.00 (2H, t, J=6.3 Hz), 2.79 (2H, t, J=5.3 Hz), 2.74 (2H, t, J=6.3 Hz), 2.36 (6H, s), 1.98 (6H, s). ¹³C NMR (CD₃OD, 100 MHz, δ, ppm) 162.93 (d, J=243.1 Hz), 158.24, 156.90 (d, J=9.9 Hz), 139.36, 131.26 (d, J=9.9 Hz), 128.70, 126.75 (d, J=3.4 Hz), 121.77, 115.68, 112.19, 111.13, 106.43 (d, J=21.2 Hz), 99.95 (d, J=25.4 Hz), 68.25, 64.76, 57.72, 44.30, 22.52. HRMS (ESI), found 398.2244 C₂₂H₂₈N₆O, [M+H]⁺, requires 398.2244.

### 1,3,5-trimethyl-4-(2-(naphthalen-2-yloxy)ethyl)-1H-pyrazole (27)

Following general procedure C, 1 (130 mg, 0.85 mmol) was reacted with 1-bromo-2-naphthol (238 mg, 1.07 mmol). The crude was purified by flash column chromatography by elution with Pentane/EtOAc in gradient to afford 200 mg (64%) of the title compound as a yellow pale solid. ¹H NMR (CDCl₃, 400 MHz, δ, ppm) 8.24-8.20 (1H, m), 7.78 (1H, d, J=2.6 Hz), 7.77-7.75 (1H, m), 7.56 (1H, ddd, J=8.4, 6.9, 1.3 Hz), 7.39 (1H, ddd, J=8.1, 6.8, 1.1 Hz), 7.19 (1H, d, J=9.0 Hz), 4.16 (2H, t. J=7.2 Hz), 3.71 (3H, s), 2.94 (2H, t, J=7.1 Hz), 2.25 (3H, s), 2.24 (3H, s).

### 3-(2-(naphthalen-2-yloxy)ethyl)pyridine (28)

Following general procedure C, 3-(2-hydroxyethyl)pyridine (55 mg, 0.44 mmol) was reacted with 1-bromo-2-naphthol (124 mg, 0.55 mmol). The crude was purified by flash column chromatography by elution with Pentane/EtOAc in gradient to afford 130 mg (89%) of the title compound as a yellow pale solid. ¹H NMR (CDCl₃, 400 MHz, δ, ppm) 8.61 (1H, d, J=1.9 Hz), 8.47 (1H, dd, J=4.8, 1.6 Hz), 8.20-8.16 (1H, m), 7.73-7.67 (3H, m), 7.51 (1H, ddd, J=8.4, 6.9, 1.2 Hz), 7.34 (1H, ddd, J=8.1, 6.9, 1.1 Hz), 7.22-7.18 (1H, m), 7.08 (1H, d, J=9.0 Hz), 4.25 (2H, t, J=6.4 Hz), 3.10 (2H, t, J=6.3 Hz).

### 3,5-dimethyl-4-(2-(naphthalen-2-yloxy)ethyl)-1H-pyrazole (29)

Following general procedure C, 2 (132 mg, 0.94 mmol) was reacted with 1-bromo-2-naphthol (238 mg, 1.07 mmol). The crude was purified by flash column chromatography by elution with Pentane/EtOAc in gradient to afford 213 mg (65%) of the title compound as a yellow pale solid. ¹H NMR (CDCl₃, 400 MHz, δ, ppm) 8.22 (1H, dt, J=8.6, 0.9 Hz), 7.77 (2H, d, J=8.9 Hz), 7.59-7.53 (1H, m), 7.44-7.36 (1H, m), 7.19 (1H, d, J=9.0 Hz), 4.18 (2H, t, J=7.1 Hz), 2.97 (2H, t, J=7.1 Hz), 2.31 (6H, s).

### 1-(4-(2-(2-(1,3,5-trimethyl-1H-pyrazol-4-yl)ethoxy) naphthalen-1-yl)pyridin-2-yl)piperazine (30a)

Following general procedure D, 27 (90 mg, 0.25 mmol) was reacted with 2-(4-tert-butoxycarbonylpiperazin-1-yl)pyridine-4-boronic acid, pinacol ester (117 mg, 0.30 mmol). The crude was purified by reverse phase chromatography (6 g C18 column; MeCN in water 0-100%). The resulting residue was reacted according to general procedure E. Afforded the title compound as a fluffy pale yellow solid (14 mg, 36%). ¹H NMR (CD₃OD, 400 MHz, δ, ppm) 8.18 (1H, d, J=5.7 Hz), 7.95 (1H, d, J=9.1 Hz), 7.83 (1H, d, J=7.1 Hz), 7.45 (1H, d, J=9.1 Hz), 7.38-7.32 (3H, m), 7.16 (1H, brs), 6.85 (1H, d, J=5.6 Hz), 4.20-4.14 (2H, m), 3.96 (4H, brs), 3.78 (3H, s), 3.42 (4H, brs), 2.79 (2H, t, J=5.8 Hz), 2.11 (3H, s), 2.06 (3H, s). ¹³C NMR (CD₃OD, 100 MHz, δ, ppm) 156.37, 154.07, 153.83, 145.48, 143.23, 142.18, 133.38, 132.13, 130.58, 129.31, 128.33, 125.27, 125.02, 123.19, 119.05, 115.99, 115.89, 114.34, 70.47, 44.30, 44.02, 35.77, 24.19, 10.40, 9.42. HRMS (ESI), found 442.2606 C₂₇H₃₁N₅O, [M+H]⁺, requires 442.2607.

### N,N-dimethyl-2-(3-(2-(2-(1,3,5-trimethyl-1H-pyrazol-4-yl)ethoxy) naphthalen-1-yl)phenoxy)ethan-1-amine (30b)

Following general procedure D, 27 (18 mg, 0.05 mmol) was reacted with 3-hydroxyphenylboronic acid (9 mg, 0.06 mmol). The crude was purified by flash column chromatography by elution with Pentane/EtOAc in gradient. The resulting residue was reacted (10 mg, 0.02 mmol) with 2-(dimethylamino)ethanol (4 mg, 0.04 mmol) according to general procedure C. The crude was purified by reverse phase chromatography (6 g C18 column; MeCN in water 0-100%) to afford the title compound as a brown resin (8 mg, 67%). 7.85 (1H, d, J=9.0 Hz), 7.83-7.77 (1H, m), 7.40-7.33 (3H, m), 7.32-7.25 (2H, m), 7.04-6.99 (1H, m), 6.82-6.78 (2H, m), 4.09 (2H, td, J=5.6, 1.4 Hz), 4.03 (2H, t, J=6.3 Hz), 3.60 (3H, s), 2.78 (2H, t, J=5.5 Hz), 2.64 (2H, t, J=6.4 Hz), 2.34 (6H, s), 1.97 (3H, s), 1.87 (3H, s). ¹³C NMR (CD₃OD, 100 MHz, δ, ppm) 160.09, 154.48, 146.78, 139.60, 139.28, 134.92, 130.69, 130.22, 128.89, 127.19, 126.06, 124.61, 118.13, 116.52, 114.33, 113.81, 71.13, 66.59, 59.15, 45.88, 35.67, 24.88, 11.48, 9.21. HRMS (ESI), found 444.2651 C₂₈H₃₃N₃O₂, [M+H]⁺, requires 444.2651.

### 1-(4-(2-(2-(pyridin-3-yl)ethoxy) naphthalen-1-yl)pyridin-2-yl)piperazine (30c)

Following general procedure D, 28 (89 mg, 0.27 mmol) was reacted with 2-(4-tert-butoxycarbonylpiperazin-1-yl)pyridine-4-boronic acid, pinacol ester (131 mg, 0.34 mmol). The crude was purified by reverse phase chromatography (6 g C18 column; MeCN in water 0-100%). The resulting residue was reacted according to general procedure E. Afforded the title compound as a fluffy pale yellow solid (60 mg, 88%). ¹H NMR (CD₃OD, 400 MHz, δ, ppm) 8.77 (1H, brs), 8.71 (1H, brs), 8.45 (1H, d, J=8.1 Hz), 8.15 (1H, d, J=6.2 Hz), 8.04-7.98 (2H, m), 7.88 (1H, dd, J=7.5, 1.4 Hz), 7.52 (1H, d, J=9.2 Hz), 7.46-7.37 (3H, m), 7.33 (1H, brs), 6.92 (1H, d, J=6.1 Hz), 4.49 (2H, q, J=5.8 Hz), 4.08-4.01 (4H, m), 3.53-3.46 (4H, m), 3.31-3.27 (2H, m). ¹³C NMR (CD₃OD, 100 MHz, δ, ppm) 155.53, 154.60, 153.56, 148.66, 142.75, 141.29, 140.86, 139.09, 133.09, 132.71, 130.64, 129.45, 128.69, 128.26, 125.48, 124.81, 122.17, 118.98, 115.66, 115.31, 69.43, 44.52, 43.80, 33.52. HRMS (ESI), found 411.2185 C₂₆H₂₆N₄O, [M+H]⁺, requires 411.2185.

### N,N-dimethyl-2-(3-(2-(2-(pyridin-3-yl)ethoxy) naphthalen-1-yl)phenoxy)ethan-1-amine (30d)

Following general procedure D, 28 (100 mg, 0.30 mmol) was reacted with 3-hydroxyphenylboronic acid (52 mg, 0.38 mmol). The crude was purified by flash column chromatography by elution with Pentane/EtOAc in gradient. The resulting residue was reacted (90 mg, 0.26 mmol) with 2-(dimethylamino)ethanol (29 mg, 0.33 mmol) according to general procedure C. The crude was purified by reverse phase chromatography (6 g C18 column; MeCN in water 0-100%) to afford the title compound as a brown resin (85 mg, 79%). ¹H NMR (CD₃OD, 400 MHz, δ, ppm) 8.17 (1H, dd, J=4.9, 1.5 Hz), 8.08 (1H, d, J=1.7 Hz), 7.66 (1H, d, J=8.9 Hz), 7.64-7.60 (1H, m), 7.32-7.28 (1H, m), 7.26-7.21 (1H, m), 7.16-7.11 (4H, m), 6.99 (1H, ddd, J=7.8, 4.9, 0.7 Hz), 6.89 (1H, ddd, J=8.3, 2.6, 0.9 Hz), 6.67 (1H, dd, J=2.5, 1.4 Hz), 6.63 (1H, dt, J=7.5, 1.2 Hz), 3.99 (2H, t, J=5.9 Hz), 4.00-3.85 (2H, m), 2.69 (2H, t, J=5.8 Hz), 2.57 (2H, t, J=5.5 Hz), 2.15 (6H, s). ¹³C NMR (CD₃OD, 100 MHz, δ, ppm) 159.93, 153.98, 150.33, 147.71, 139.18, 139.11, 136.59, 134.68, 130.51, 130.28, 130.22, 128.94, 127.30, 126.65, 126.04, 124.76, 124.68, 124.63, 118.19, 115.75, 114.20, 70.29, 66.47, 59.02, 45.85, 33.87. HRMS (ESI), found 413.2229 C₂₇H₂₈N₂O₂, [M+H]⁺, requires 413.2229.

### 1-(4-(2-(2-(3,5-dimethyl-1H-pyrazol-4-yl)ethoxy) naphthalen-1-yl)pyridin-2-yl)piperazine (30e)

Following general procedure D, 29 (40 mg, 0.11 mmol) was reacted with 2-(4-tert-butoxycarbonylpiperazin-1-yl)pyridine-4-boronic acid, pinacol ester (56 mg, 0.14 mmol). The crude was purified by reverse phase chromatography (6 g C18 column; MeCN in water 0-100%). The resulting residue was reacted according to general procedure E. Afforded the title compound as a fluffy pale yellow solid (12 mg, 50%). ¹H NMR (CD₃OD, 400 MHz, δ, ppm) 8.21 (1H, d, J=6.4 Hz), 8.05 (1H, d, J=9.1 Hz), 7.92-7.88 (1H, m), 7.54 (1H, d, J=9.2 Hz), 7.52 (1H, brs), 7.50-7.39 (3H, m), 7.09 (1H, dd, J=6.4, 1.1 Hz), 4.32 (2H, q, J=6.3 Hz), 4.18-4.11 (4H, m), 3.58-3.52 (4H, m), 2.94 (2H, t, J=6.2 Hz), 2.29 (6H, s). ¹³C NMR (CD₃OD, 100 MHz, δ, ppm) 157.09, 153.95, 153.61, 145.53, 137.48, 132.97, 132.88, 130.51, 129.43, 128.76, 125.47, 124.76, 121.73, 119.16, 116.71, 116.45, 115.54, 69.69, 44.71, 43.67, 23.33, 9.70. HRMS (ESI), found 428.2450 C₂₆H₂₉N₅O, [M+H]⁺, requires 428.2450.

### 2-(3-(2-(2-(3,5-dimethyl-1H-pyrazol-4-yl)ethoxy) naphthalen-1-yl)phenoxy)-N,N-dimethylethan-1-amine (30f)

Following general procedure D, 29 (100 mg, 0.28 mmol) was reacted with 3-hydroxyphenylboronic acid (48 mg, 0.34 mmol). The crude was purified by flash column chromatography by elution with Pentane/EtOAc in gradient. The resulting residue was reacted (100 mg, 0.28 mmol) with 2-(dimethylamino)ethanol (30 mg, 0.33 mmol) according to general procedure C. The crude was purified by reverse phase chromatography (6 g C18 column; MeCN in water 0-100%) to afford the title compound as a brown resin (70 mg, 60%). ¹H NMR (CDCl₃, 400 MHz, δ, ppm) 7.83 (1H, d, J=9.1 Hz), 7.81-7.77 (1H, m), 7.44-7.40 (1H, m), 7.39-7.34 (1H, m), 7.32-7.28 (3H, m), 6.93-6.87 (2H, m), 6.56 (1H, dd, J=2.5, 1.5 Hz), 4.23-4.17 (1H m), 4.12 (1H, ddd, J=10.7, 7.7, 3.3 Hz), 4.01 (2H, ddd, J=8.1, 6.4, 3.7 Hz), 2.93 (1H, ddd, J=13.3, 7.6, 3.6 Hz), 2.74-2.68 (3H, m), 2.46 (6H, s), 1.96 (6H, s). ¹³C NMR (CDCl₃, 100 MHz, δ, ppm) 158.64, 153.15, 137.97, 133.70, 129.05, 128.89, 128.77, 127.77, 126.21, 125.40, 125.11, 123.58, 123.48, 117.97, 114.53, 111.74, 111.33, 69.18, 65.18, 58.59, 45.82, 23.55, 10.81. HRMS (ESI), found 430.2496 C₂₇H₃₁N₃O₂, [M+H]⁺, requires 430.2495.

TABLE 4
X-ray diffraction and structure refinement statistics.

| PDB entry | |
|---|---|
| SSGCID sample ID | PlviB.18219.a.FR2.GE44010 |
| X-ray source | ALS beamline 8.2.2 |
| Wavelength (Å) | 1.00000 |
| Resolution (Å) | 50.00-1.65 |
| Space group | P4₃2₁2 |
| Unit cell | |
| a, c (Å) | 91.930, 101.286 |
| No. reflections | 751,443 |
| Unique reflections | 52,868 |
| R_{merge} (%) | 11.6 (88.9) |
| Rₚᵢₘ (%) | 3.2 (24.6) |
| CC½ | 0.998 (0.903) |
| Completeness (%) | 100.0 (100.0) |
| Redundancy | 14.2 (13.4) |
| <I>/<σ> | 23.7 (2.2) |
| Refinement | |
| Resolution (Å) | 45.97-1.65 |
| No. reflections | 52,787 |
| Protein atoms | 6,928 |
| Ligand atoms | 402 |
| Water molecules | 435 |
| R_{work}/R_{free} (%) | 13.61/16.48 |
| RMS deviations | |
| Bond lengths (Å) | 0.010 |
| Bond angles (°) | 1.085 |
| Ramachandran plot | |
| Favored (%) | 97.13 |
| Allowed (%) | 2.87 |
| Outlier (%) | 0.00 |
| Molprobity | |
| All-atom clashscore | 1.91 |

*Values in parentheses correspond to the highest resolution shell

TABLE 5
Effect of NMT inhibitors of Formula (I) on P. vivax liver stage infection

| | schizonts (fold change to DMSO) | | | | | | | hypnozoites (fold change to DMSO) | | | | | |
|---|---|---|---|---|---|---|---|---|---|---|---|---|---|
| | isolate 1 | | isolate 2 | | isolate 3 | | difference | isolate 1 | | isolate 2 | | difference | difference |
| | mean | | mean | | mean | | between | mean | | mean | | between | between |
| compound | diff | p-value | diff | p-value | diff | p-value | isolates? | diff | p-value | diff | p-value | isolates? | forms? |
| 12a | 0.850 | 0.000 | 0.9245 | <0.0001 | 0.7273 | <0.0001 | no | 0.754 | 0.0015 | 0.897 | <0.0001 | no | no |
| 12b | 0.950 | <0.0001 | 0.8868 | <0.0001 | 0.8977 | <0.0001 | no | 0.923 | <0.0001 | 0.966 | <0.0001 | no | no |
| 12d | 0.967 | <0.0001 | 0.9434 | <0.0001 | 0.8977 | <0.0001 | no | 0.939 | <0.0001 | 0.862 | <0.0001 | no | no |
| 12e | 0.983 | <0.0001 | 0.8302 | <0.0001 | 0.8295 | <0.0001 | no | 0.815 | 0.0005 | 1.000 | <0.0001 | no | no |
| 12f | 1.000 | <0.0001 | 0.9811 | <0.0001 | 0.8295 | <0.0001 | no | 0.939 | <0.0001 | 0.931 | <0.0001 | no | no |
| 12g | 1.000 | <0.0001 | 0.7547 | <0.0001 | 0.7955 | <0.0001 | no | 0.785 | 0.0009 | 0.966 | <0.0001 | no | no |
| 12j | 0.983 | <0.0001 | 0.9434 | <0.0001 | 0.7614 | <0.0001 | no | 0.985 | <0.0001 | 0.897 | <0.0001 | no | no |
| 12l | 0.983 | <0.0001 | 0.7736 | <0.0001 | 0.6932 | <0.0001 | no | 1.000 | <0.0001 | 0.828 | <0.0001 | no | no |
| 12q | 0.983 | <0.0001 | 0.8491 | <0.0001 | 0.6591 | <0.0001 | yes | 0.939 | <0.0001 | 0.931 | <0.0001 | no | no |
| 26b | 0.917 | <0.0001 | 0.8679 | <0.0001 | 0.625 | <0.0001 | no | 0.908 | <0.0001 | 0.793 | <0.0001 | no | no |
| 26c | 1.000 | <0.0001 | 0.9434 | <0.0001 | 0.8977 | <0.0001 | no | 0.923 | <0.0001 | 0.862 | <0.0001 | no | no |
| 26i | 0.933 | <0.0001 | 0.8491 | <0.0001 | 0.8295 | <0.0001 | no | 1.000 | <0.0001 | 0.310 | 0.0429 | yes | yes |
| 30a | 1.000 | <0.0001 | 0.9245 | <0.0001 | 0.8636 | <0.0001 | no | 0.954 | <0.0001 | 1.000 | <0.0001 | no | no |
| 30c | 0.900 | 0.000 | 0.7547 | <0.0001 | 0.8636 | <0.0001 | no | 0.939 | <0.0001 | 0.690 | <0.0001 | no | no |

### Study Design

This study aimed to identify NMT is with high selectivity for the P. vivax enzyme compared with human NMTs and high potency against the different stages of the P. vivax parasite. For this purpose, a structure-guided approach was applied using previously reported NMT inhibitors as scaffolds to develop a new generation of PvNMT-targeting compounds. The NMT activity of these inhibitors was first measured using biochemical assays through the detection of free CoA by the thiol-reactive probe 7-diethylamino-3-(4′-maleimidylphenyl)-4-methylcoumarin (CPM). Their antiparasitic activity were further evaluated by measuring blood-stage parasite load and inhibition of P. vivax liver-stage schizont and hypnozoite infection. X-ray co-crystallization of PvNMT with a representative lead compound, 12b, was used to rationalize the observed selectivity for PvNMT over HsNMT. Cytotoxicity was assessed in the human hepatoma HepG2 cell line. Synchronized P. falciparum NF54 ring-stage parasites were used for measuring blood-stage parasite load. P. vivax sporozoites were obtained from three independent patient isolates. Liver-stage infections were cultured in primary human hepatocytes from a single donor lot using a 384-well microculture system. In vitro experiments were repeated at least twice. A single concentration of each inhibitor was initially used to evaluate the inhibition of P. vivax liver stage schizont and hypnozoite infection, followed by the determination of the dose-response relationship.

### Docking of Compounds (12e) in the PvNMT (PDB: 6MB1) Model Binding-Pocket

The hybrid compound 12e was rendered in the form of 2D images using the ChemDraw Professional (Version 19.1.1.21) software package, converted to SDF format, and then prepared for docking using the Molecular Operating Environment (MOE 2019.01) software package. After loading the SDF files, it was processed as follows: the compound was energy-minimized and partial charges added (Amber10 forcefield) using QuickPrep. To prepare the receptor protein, the PvNMT PDB file was loaded into MOE and processed using QuickPrep. The docking simulation was set up by setting the receptor to “receptor+solvent”. The SDF file containing the processed ligands to be docked was loaded. Ligand placement and refinement were performed using the Alpha PMI and rigid receptor methods, with 30 and 3 poses, respectively.

Cloning, Expression and Purification of PvNMT P. vivax and H. sapiens NMT Enzymes Cloning, Expression and Purification of PvNMT

Cloning, expression and purification were conducted as part of the Seattle Structural Genomics Center for Infectious Disease (SSGCID) following protocols described previously. A region of the PvNMT gene encoding residues 27-410 with a N-terminus 6×His sequence and PreScission cleavage site was cloned into a pET11a expression vector. The N-terminal sequence is MGSSHHHHHHSAALEVLFQ/GP-ORF, where cleavage occurs between the glutamine and glycine residues. Plasmid DNA was transformed into chemically competent E. coli Rosetta 2 (DE3) pRARE cells. Cells were expression tested and 4-12 liters of culture were grown using auto-induction media in the LEX bioreactor for 18-22 h at 18° C. The expression clone was assigned the SSGCID target identifier PlviB.18219.a.FR2.GE44010 and is available at https://www.ssgcid.org/available-materials/ssgcid-proteins/. Protein was purified following a 5-step procedure as previously described consisting of a Ni²⁺-affinity chromatography (IMAC), cleavage of the 6×His-tag and pass through over a second Ni²⁺-affinity chromatography (IMAC) column to remove cleaved tag and protease. The eluted protein was purified further using an anion exchange HiTRAP Q HP 5 ml column. Peak fractions were concentrated to 5 mL and applied to a Superdex 75 10/300 column. The final buffer was composed of 0.3 M NaCl, 20 mM HEPES, 5% (v/v) glycerol, 1 mM TCEP, pH 7.0. Fractions were analyzed on an SDS-PAGE gel and fractions containing the target protein were concentrated and flash frozen and stored at −80° C. until further use.

Crystallization and Structure Determination of P. vivax NMT

Purified PvNMT (27-410) concentrated to 8 mg/ml was incubated with 1 mM myrCoA (MedChem101 LLC.) and compound 12b for 20 minutes at room temperature and set up in 96-well sitting drop crystallization screens JCSG+HT96 (Molecular Dimensions) and Morpheus HT96 (Molecular Dimensions). Crystals formed within 2 weeks in JCSG+condition A1 composed of 0.2 M lithium sulfate, 0.1 M sodium acetate, and 50% PEG 400, pH 4.5. Crystals were harvested directly and flash frozen in liquid nitrogen without cryo-protectant exchange. Frozen crystals were shipped to the Advanced Light Source (ALS), Berkeley National Laboratory as part of the Collaborative Crystallography program of ALS-ENABLE. Data were collected at 100° K on ALS-ENABLE beamlines as described in Table 4. Raw X-ray diffraction images are available at the Integrated Resource for Reproducibility in Macromolecular Crystallography at www.proteindiffraction.org. Data were indexed and integrated with HKL2000 and scaled with XSCALE. The structure was solved with Phaser using PDB 6NXG as a search model. The model was refined with iterative rounds of refinement with Phenix and manual model building in Coot. The quality of the structure was checked with Molprobity.

### NMT Activity Assay

To measure the activity of the purified PvNMT an assay was adapted from the literature. The assay buffer was prepared in a 4x stock solution consisting of 9.2 mM potassium phosphate, 69.7 mM sodium phosphate, 2 mM EDTA and 10% TritonX-100 at pH 7.0. Working stock solutions were made fresh adding DMSO for final concentrations of either 1% or 5% DMSO. The PvNMT enzyme was diluted in assay buffer containing 1% DMSO for a final concentration of 25 nM. Ten (10) μL of the test compound or 10% (v/v) DMSO/water were dispensed into a 96 well plate (Greiner Bio-One) and 50 μL of enzyme (in assay buffer containing 1% DMSO) were added for a final concentration of 25 nM per well. The plate was incubated for 30 min at room temperature. The enzymatic reaction was initiated by adding 50 μL of reaction substrate containing 10 UM myrCoA and PfARF, as well as 8 μM CPM. Fluorescent readings were taken on a Spectra M2 plate reader (Molecular Devices) with excitation at 385 nm and emission at 485 nm. Fluorescent intensity was measured continuously in one-minute intervals for 45 minutes. Background fluorescence and noise were determined by replacing each constituent of the reaction individually with assay buffer containing 1% DMSO and values were deducted from experimental samples. The enzymatic reactions were set up with varying pHs (6.0-8.5) of the assay buffer to determine optimal reaction conditions with minimal background and off target reactions. In lieu of compound, 10 μL of 10% DMSO/H₂O was added to each well. Fluorescence signal was obtained continuously for 45 min. A linear reaction rate was observed during the first 30 minutes and used to determine all values. The synthetic peptide (PfARF) Gly-Leu-Tyr-Val-Ser-Arg-Leu-Phe-Asn-Arg-Leu-Phe-Gln-Lys-Lys-NH₂ (amidated at the C-terminus) was purchased from Innopep (San Diego, California). 7-Diethylamino-3-(4′-Maleimidylphenyl)-4-Methylcoumarin (CPM) was purchased from Thermo Scientific Life Technologies (Grand Island, New York) and the co-factor myrCoA was purchased from Med Chem 101 LLC (Plymouth Meeting, Pennsylvania). IC₅₀ calculations were calculated using Prism (GraphPad Software, Inc).

### HepG2 Cytotoxicity Assay

Viability of the HepG2 cell line following exposure to the compounds was determined by a Live/Dead cell assay kit (Invitrogen). Briefly, cells were added to a 96-well plate at a concentration of 5000 cells per well, excluding the exterior wells. Cells were exposed to the compounds at different concentrations ranging from 1 to 20 μM. Cells were washed every 24 h and supplemented with fresh doses of compounds. At 72 h post treatment, 1 μM calcein-AM and 1 μM ethidium homodimer were added to the wells and incubated for 10 min at 37° C. After washing with PBS, the cells were visualized with fluorescent microscopy (Keyence BZ-X700).

P. falciparum Blood Stage Assay.

Sorbitol-synchronized P. falciparum NF54 ring-stage parasites were cultured at a parasitemia of 0.5% and hematocrit of 1.5% in a 96-well microtiter plate. Parasites were treated with each NMT inhibitors compounds at 0.625 μM, 1.25 μM, 2.5 μM, 5 μM or 10 μM for 72 h. Direct lysis of the blood cells to release the parasite DNA was performed by adding 100 μL of LBS buffer containing SYBR green I DNA binding dye to each well. Plates were incubated at-20° C. overnight for complete lysis and fluorescence was measured at 485 nm (excitation) and 528 nm (emission). ICEstimator regression analysis (antimalarial-icestimator dot net) was used to obtain relative IC₅₀ values for each compound.

P. vivax Liver Stage Assay

Plasmodium vivax liver stage assays were performed as described previously. Briefly, 384-well plates were seeded with primary hepatocytes from a single donor lot at a density of 25,000 cells per well. The hepatocytes were then infected with 14,000 freshly hand-dissected sporozoites per well. Cells were exposed to the compounds at indicated concentrations from day 3 post-infection, then proceeded to feed cultures every day, replacing the compounds until day 8 post-infection. Cells were fixed with 4% PFA and stained with PvUIS4 antibodies to detect schizonts and hypnozoites forms by confocal microscopy. The images were processed and analyzed using IMARIS (Bitplane Inc.) image analysis software.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following enumerated embodiments are provided, the numbering of which is not to be construed as designating levels of importance:

Embodiment 1 provides a compound of Formula (I), or a pharmaceutically acceptable salt or solvate thereof:

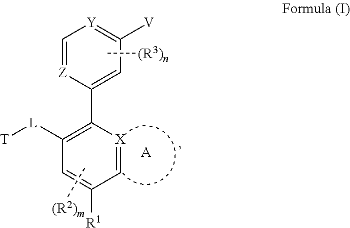

wherein
A is an optionally fused 6-membered aryl or heteroaryl ring, which is optionally substituted by at least one substituent selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂,

SO₃R, C(O)R, C(O)C(O)R, C(O)CH₂C(O)R, C(S)R, C(O)OR, OC(O)R, and C(O)N(R)₂;
X is C, CH, or N, provided that if A is present then X is C;
Z is CH or N;
Y is CH or N;
R¹ is selected from the group consisting of hydrogen, F, Cl, and Br;
L is O, NR, or ═N—*, wherein the bond with * is attached to the aryl ring;
T is —C₁₋₆ alkyl-C₅₋₆ heteroaryl, wherein the C₅₋₆ heteroaryl is optionally substituted with at least one selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
V is —C₀₋₆ alkyl-C₅₋₆ heterocyclyl, —O—C₀₋₆ alkyl-C₅₋₆ heterocyclyl, —C₀₋₆ alkyl-NR₂, or —O—C₂₋₆ alkyl-NR₂, wherein the C₅₋₆ heterocyclyl is optionally substituted with at least one selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
R is independently at each occurrence selected from the group consisting of hydrogen or optionally substituted C₁₋₁₂ alkyl;
R² is independently at each occurrence selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
R³ is independently at each occurrence selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
m is 0, 1, 2, or 3; and
n is 0, 1, 2, 3, or 4.

Embodiment 2 provides the compound of embodiment 1, which has the structure

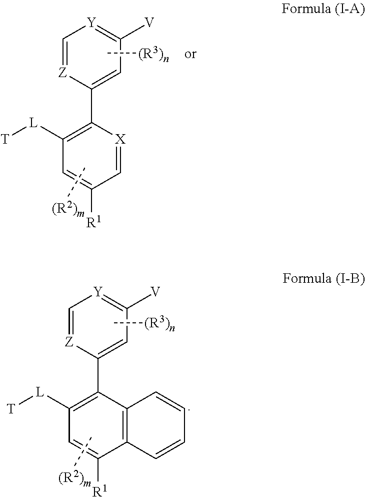

Embodiment 3 provides the compound of any one of embodiments 1-2, wherein L is O.

Embodiment 4 provides the compound of any one of embodiments 1-3, wherein R¹ is H or F.

Embodiment 5 provides the compound of any one of embodiments 1-4, wherein T is C₂ alkyl-C₅₋₆ heteroaryl.

Embodiment 6 provides the compound of any one of embodiments 1-5, wherein T is selected from the group consisting of

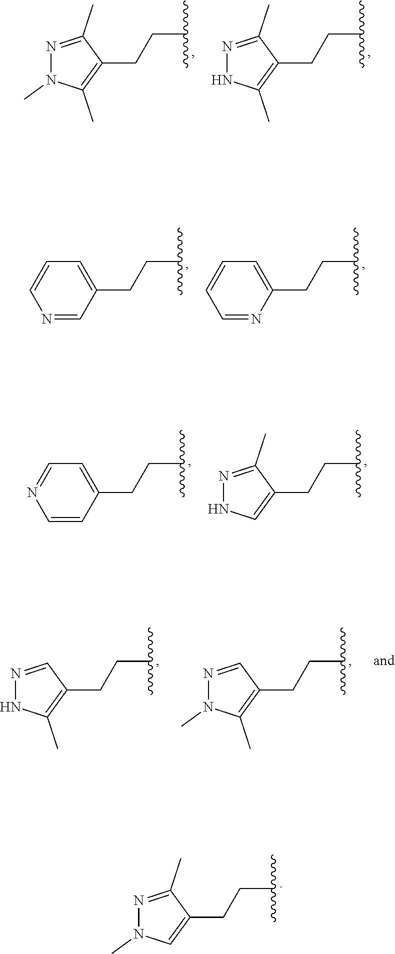

Embodiment 7 provides the compound of any one of embodiments 1-6, wherein m is 0.

Embodiment 8 provides the compound of any one of embodiments 1-7, wherein n is 0.

Embodiment 9 provides the compound of any one of embodiments 1-8, wherein V is C₅₋₆ heterocyclyl.

Embodiment 10 provides the compound of any one of embodiments 1-9, wherein V is —C₁₋₆ alkyl-C₅₋₆ heterocyclyl or —O—C₂₋₆ alkyl-NR₂.

Embodiment 11 provides the compound of any one of embodiments 1-10, wherein V is

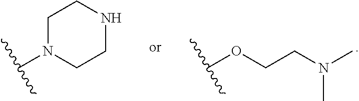

Embodiment 12 provides the compound of any one of embodiments 1-11, wherein A is

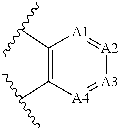

wherein each A1, A2, A3, and A4 is independently CH or N, provided no more than two of A1, A2, A3, and A4 are N; and each CH group in A1 to A4 is optionally substituted.

Embodiment 13 provides the compound of any one of embodiments 1-12, which is at least one compound selected from the group consisting of:

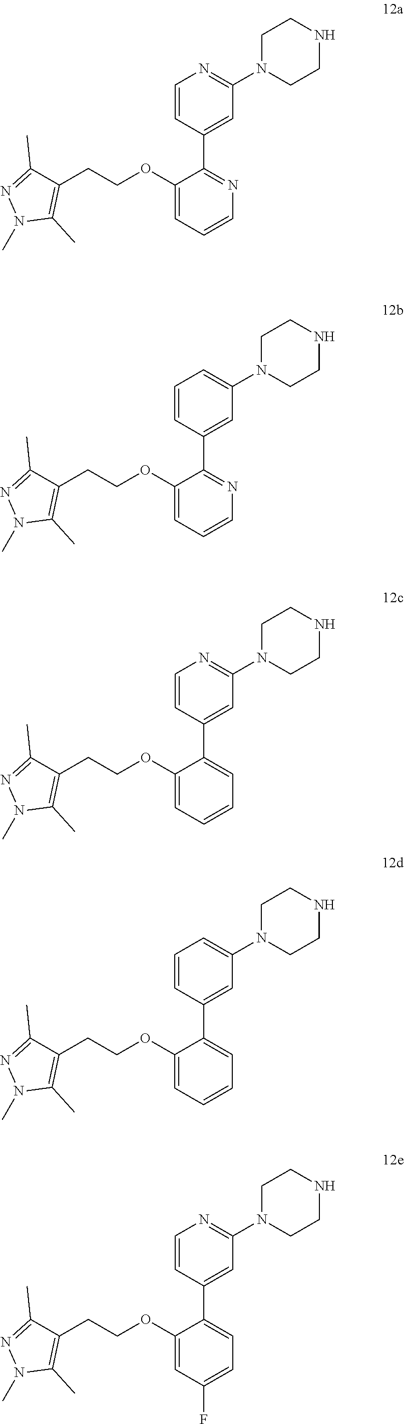 [SMILES: Cc1nn(C)c(C)c1CCOc1cccnc1-c1ccnc(N2CCNCC2)c1 ; Cc1nn(C)c(C)c1CCOc1cccnc1-c1cccc(N2CCNCC2)c1 ; Cc1nn(C)c(C)c1CCOc1ccccc1-c1ccnc(N2CCNCC2)c1 ; Cc1nn(C)c(C)c1CCOc1ccccc1-c1cccc(N2CCNCC2)c1 ; Cc1nn(C)c(C)c1CCOc1cc(F)ccc1-c1ccnc(N2CCNCC2)c1]

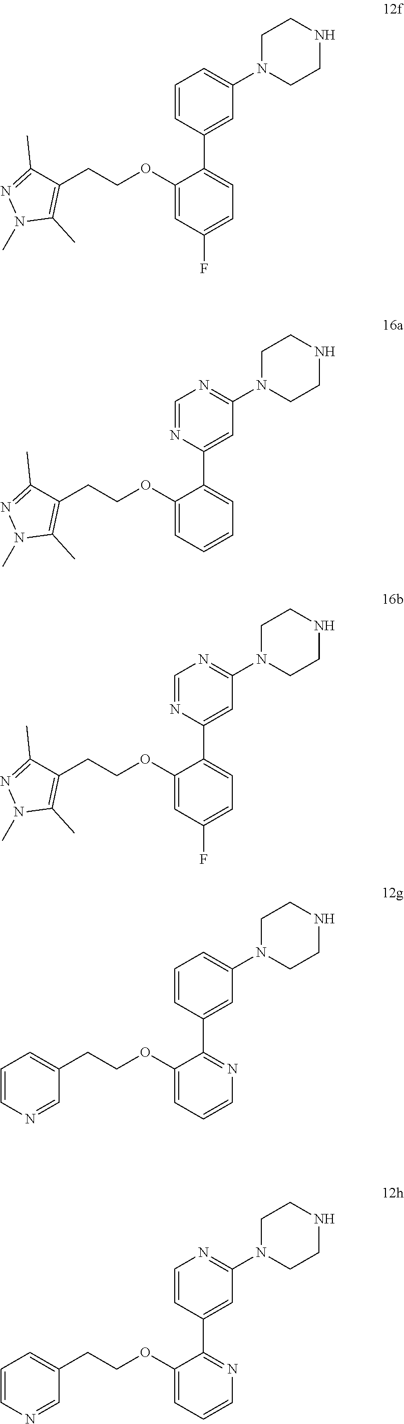 [SMILES: Cc1nn(C)c(C)c1CCOc1cc(F)ccc1-c1cccc(N2CCNCC2)c1 ; Cc1nn(C)c(C)c1CCOc1ccccc1-c1cc(N2CCNCC2)ncn1 ; Cc1nn(C)c(C)c1CCOc1cc(F)ccc1-c1cc(N2CCNCC2)ncn1 ; c1cncc(CCOc2cccnc2-c2cccc(N3CCNCC3)c2)c1 ; c1cncc(CCOc2cccnc2-c2ccnc(N3CCNCC3)c2)c1]

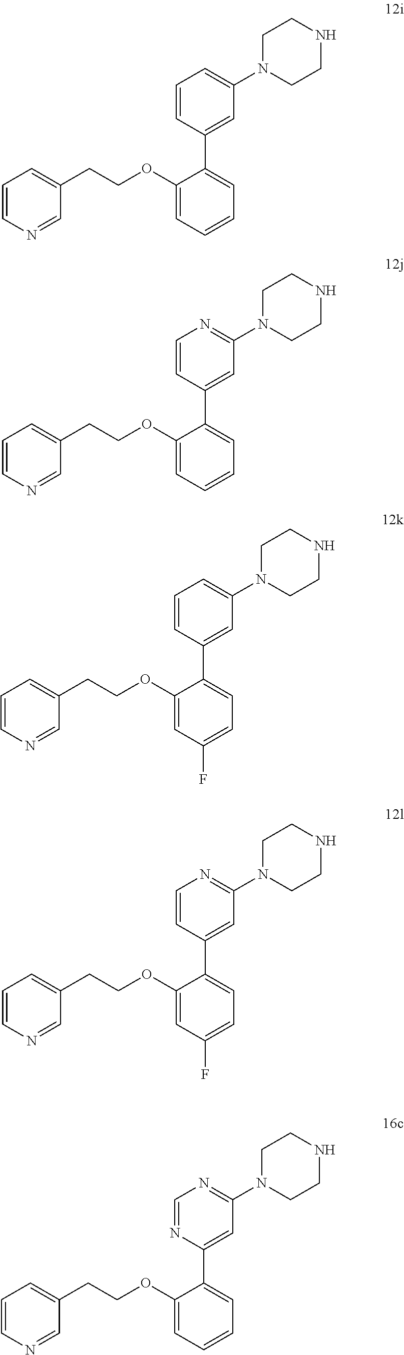 [SMILES: c1cncc(CCOc2ccccc2-c2cccc(N3CCNCC3)c2)c1 ; c1cncc(CCOc2ccccc2-c2ccnc(N3CCNCC3)c2)c1 ; Fc1ccc(-c2cccc(N3CCNCC3)c2)c(OCCc2cccnc2)c1 ; Fc1ccc(-c2ccnc(N3CCNCC3)c2)c(OCCc2cccnc2)c1 ; c1cncc(CCOc2ccccc2-c2cc(N3CCNCC3)ncn2)c1]

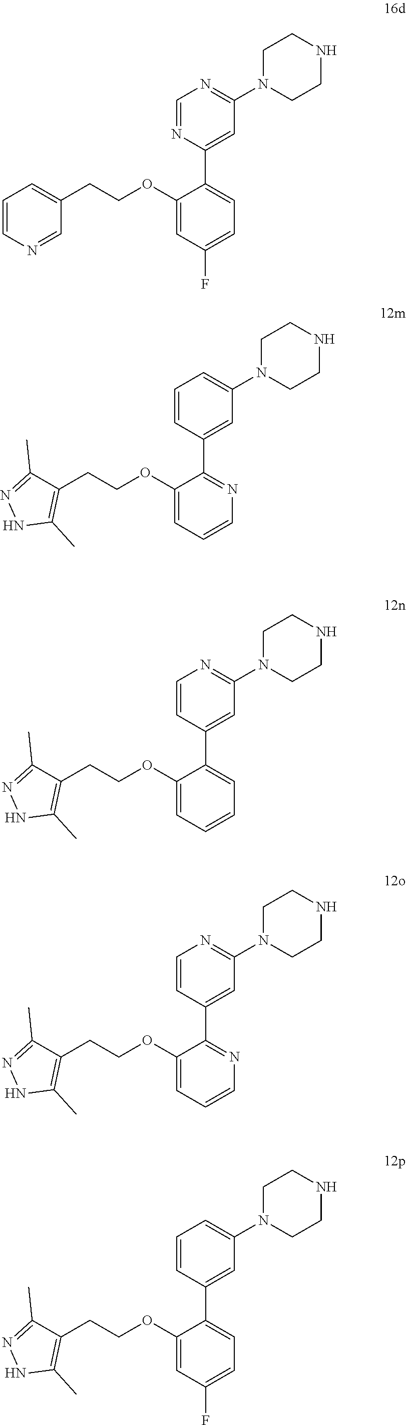 [SMILES: Fc1ccc(-c2cc(N3CCNCC3)ncn2)c(OCCc2cccnc2)c1 ; Cc1n[nH]c(C)c1CCOc1cccnc1-c1cccc(N2CCNCC2)c1 ; Cc1n[nH]c(C)c1CCOc1ccccc1-c1ccnc(N2CCNCC2)c1 ; Cc1n[nH]c(C)c1CCOc1cccnc1-c1ccnc(N2CCNCC2)c1 ; Cc1n[nH]c(C)c1CCOc1cc(F)ccc1-c1cccc(N2CCNCC2)c1]

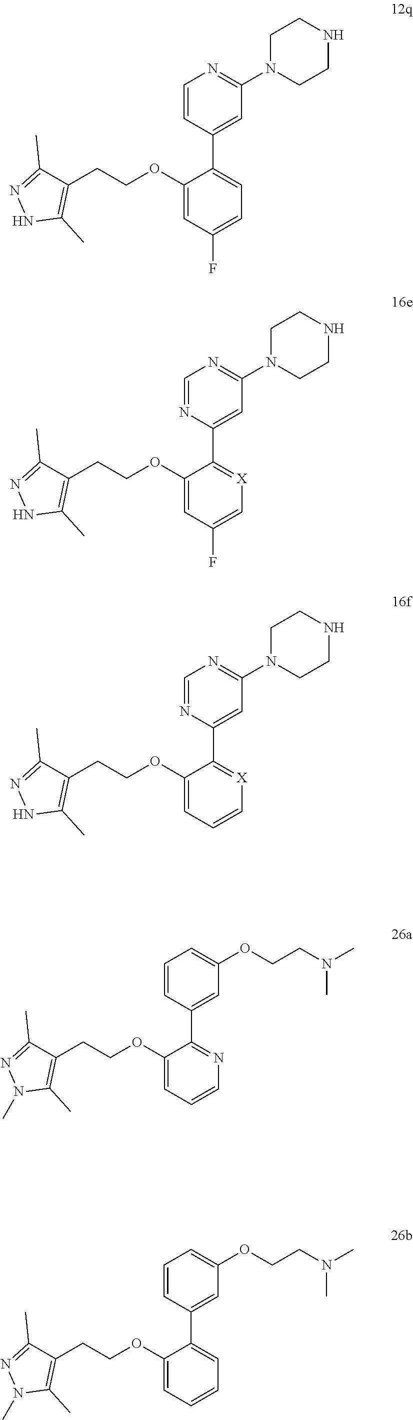

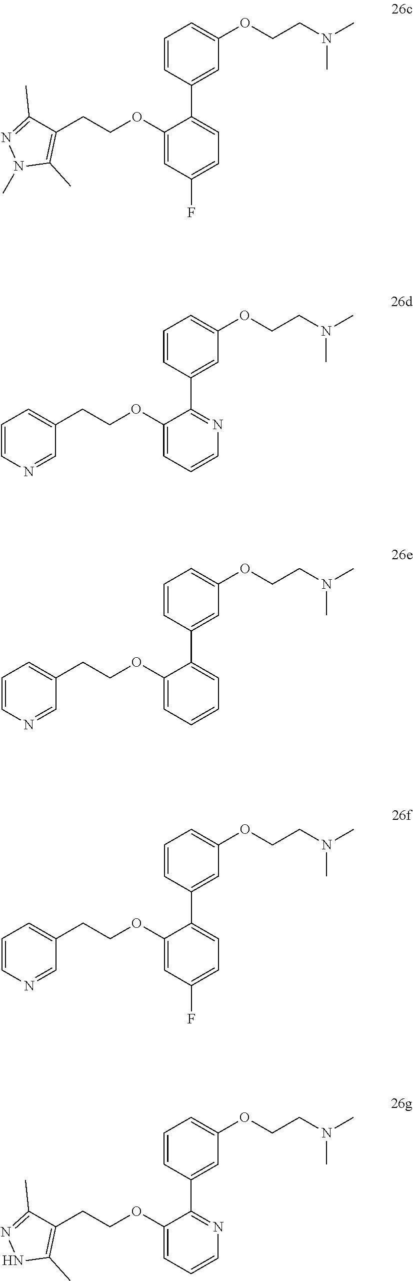

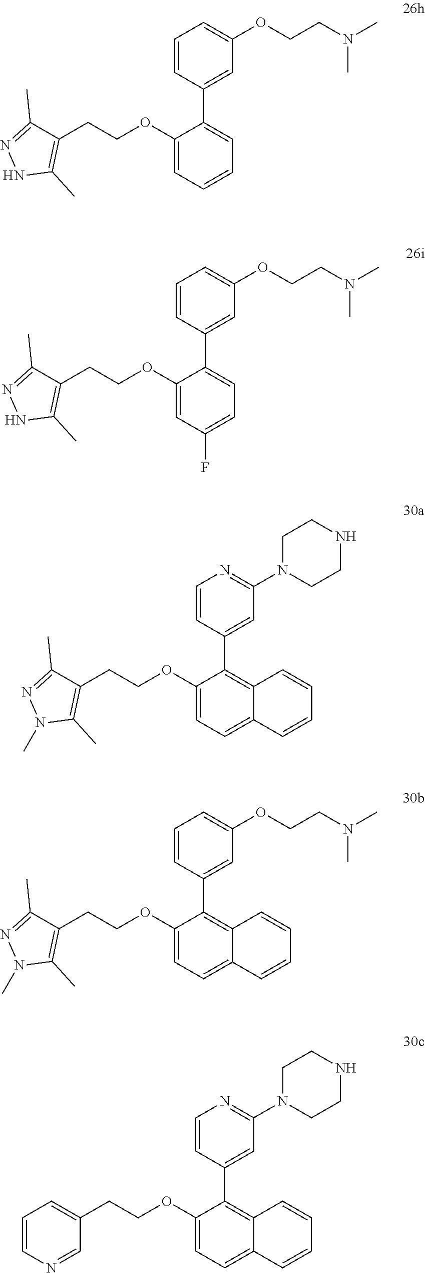

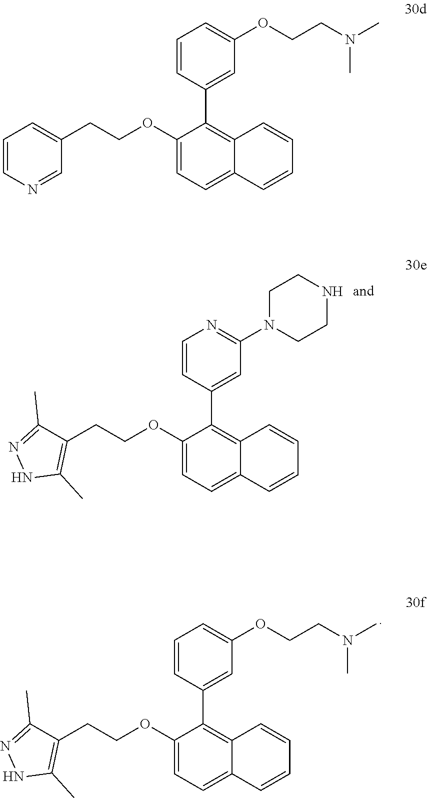 [SMILES: CN(C)CCOc1cccc(-c2c(OCCc3cccnc3)ccc3ccccc23)c1 ; Cc1n[nH]c(C)c1CCOc1ccc2ccccc2c1-c1ccnc(N2CCNCC2)c1 ; Cc1n[nH]c(C)c1CCOc1ccc2ccccc2c1-c1cccc(OCCN(C)C)c1]

Embodiment 14 provides a method of treating, ameliorating, or preventing malaria in a subject in need thereof, the method comprising: administering to the subject a therapeutically effective amount of a compound of any one of embodiments 1-13 and at least one pharmaceutically acceptable excipient or carrier.

Embodiment 15 provides the method of embodiment 14, wherein the administering is by a route selected from the group consisting of oral, buccal, transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Embodiment 16 provides the method of any one of embodiments 14-15, wherein the subject is a human.

Embodiment 17 provides the method of any one of embodiments 14-16, wherein the subject is infected by Plasmodium falciparum or Plasmodium vivax.

Embodiment 18 provides the method of any one of embodiments 14-17, wherein the Plasmodium vivax is in a dormant form (hypnozoites) in the subject.

Embodiment 19 provides a method of killing or inhibiting a Plasmodium falciparum or Plasmodium vivax parasite, the method comprising: contacting the parasite with an effective amount of the compound of any one of embodiments 1-13 sufficient to kill the parasite or render the parasite incapable of causing malaria in a subject.

Embodiment 20 provides the method of embodiment 19, wherein the Plasmodium vivax is in a dormant form.

Embodiment 21 provides a pharmaceutical composition comprising at least one compound of any one of embodiments 1-13 and at least one pharmaceutically acceptable excipient or carrier.

Embodiment 22 provides the pharmaceutical composition of embodiment 21, wherein the at least one compound is present in an amount of about 0.01 to about 5,000 mg.

Embodiment 23 provides the pharmaceutical composition of any one of embodiments 21-22, which is formulated as a tablet or capsule.

Embodiment 24 provides the pharmaceutical composition of any one of embodiments 21-22, which is a liquid formulation.

1. A compound of Formula (I), or a pharmaceutically acceptable salt or solvate thereof: 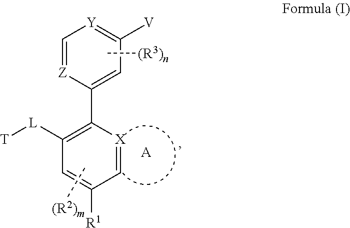
wherein
A is an optionally fused 6-membered aryl or heteroaryl ring, which is optionally substituted by at least one substituent selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
X is C, CH, or N, provided that if A is present then X is C;
Z is CH or N;
Y is CH or N;
R¹ is selected from the group consisting of hydrogen, F, Cl, and Br;
L is O, NR, or ═N—*, wherein the bond with * is attached to the aryl ring;
T is —C₁₋₆ alkyl-C₅₋₆ heteroaryl, wherein the C₅₋₆ heteroaryl is optionally substituted with at least one selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
V is —C₀₋₆ alkyl-C₅₋₆ heterocyclyl, —O—C₀₋₆ alkyl-C₅₋₆ heterocyclyl, —C₀₋₆ alkyl-NR₂, or —O—C₂₋₆ alkyl-NR₂, wherein the C₅₋₆ heterocyclyl is optionally substituted with at least one selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
R is independently at each occurrence selected from the group consisting of hydrogen or optionally substituted C₁₋₁₂ alkyl;
R² is independently at each occurrence selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
R³ is independently at each occurrence selected from the group consisting of F, Cl, Br, I, OR, OC(O)N(R)₂, CN, NO, NO₂, ONO₂, CF₃, OCF₃, R, N(R)₂, SR, SOR, SO₂R, SO₂N(R)₂, SO₃R, C(O)R, C(O)C(O)R, C(O)CH₂C(O)R, C(S)R, C(O)OR, OC(O)R, and C(O)N(R)₂;
m is 0, 1, 2, or 3; and
n is 0, 1, 2, 3, or 4.

2. The compound of claim 1, which has the structure

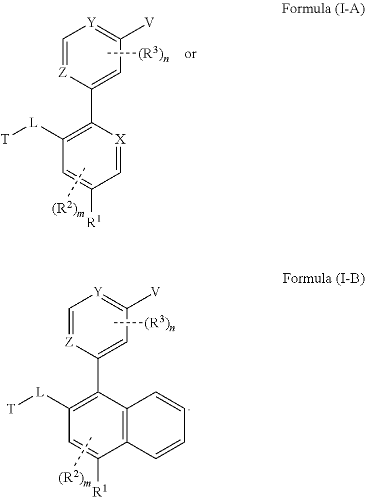

3. The compound of claim 1, wherein L is O.

4. The compound of claim 1, wherein R¹ is H or F.

5. The compound of claim 1, wherein T is C₂ alkyl-C₅₋₆ heteroaryl.

6. The compound of claim 5, wherein T is selected from the group consisting of:

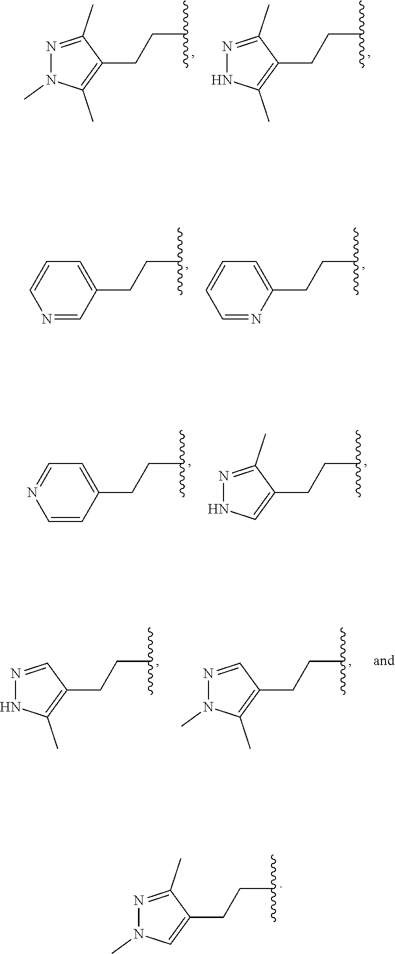

7. The compound of claim 1, wherein m is 0.

8. The compound of claim 1, wherein n is 0.

9. The compound of claim 1, wherein Vis C₅₋₆ heterocyclyl.

10. The compound of claim 9, wherein Vis-C₁₋₆ alkyl-C₅₋₆ heterocyclyl or —O—C₂₋₆ alkyl-NR₂.

11. The compound of claim 10, wherein V is

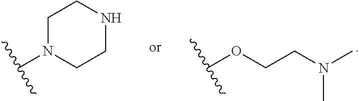

12. The compound of claim 1, wherein A is 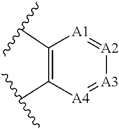
wherein
each A1, A2, A3, and A4 is independently CH or N, provided no more than two of A1, A2, A3, and A4 are N; and
each CH group in A1 to A4 is optionally substituted.

13. The compound of claim 1, which is at least one compound selected from the group consisting of:

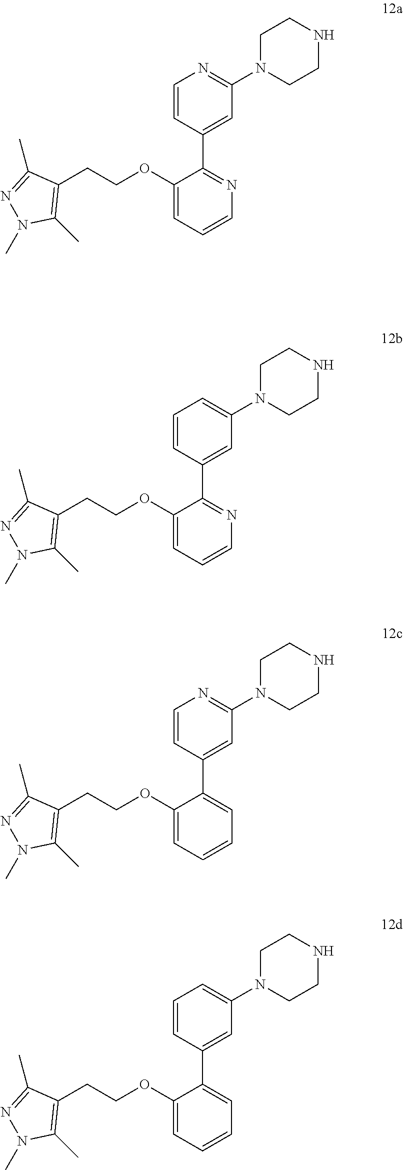 [SMILES: Cc1nn(C)c(C)c1CCOc1cccnc1-c1ccnc(N2CCNCC2)c1 ; Cc1nn(C)c(C)c1CCOc1cccnc1-c1cccc(N2CCNCC2)c1 ; Cc1nn(C)c(C)c1CCOc1ccccc1-c1ccnc(N2CCNCC2)c1 ; Cc1nn(C)c(C)c1CCOc1ccccc1-c1cccc(N2CCNCC2)c1]

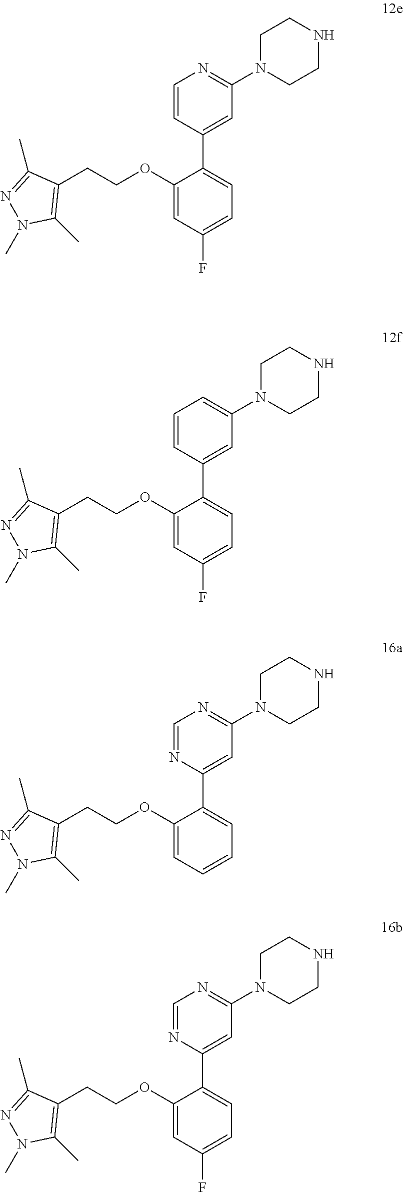 [SMILES: Cc1nn(C)c(C)c1CCOc1cc(F)ccc1-c1ccnc(N2CCNCC2)c1 ; Cc1nn(C)c(C)c1CCOc1cc(F)ccc1-c1cccc(N2CCNCC2)c1 ; Cc1nn(C)c(C)c1CCOc1ccccc1-c1cc(N2CCNCC2)ncn1 ; Cc1nn(C)c(C)c1CCOc1cc(F)ccc1-c1cc(N2CCNCC2)ncn1]

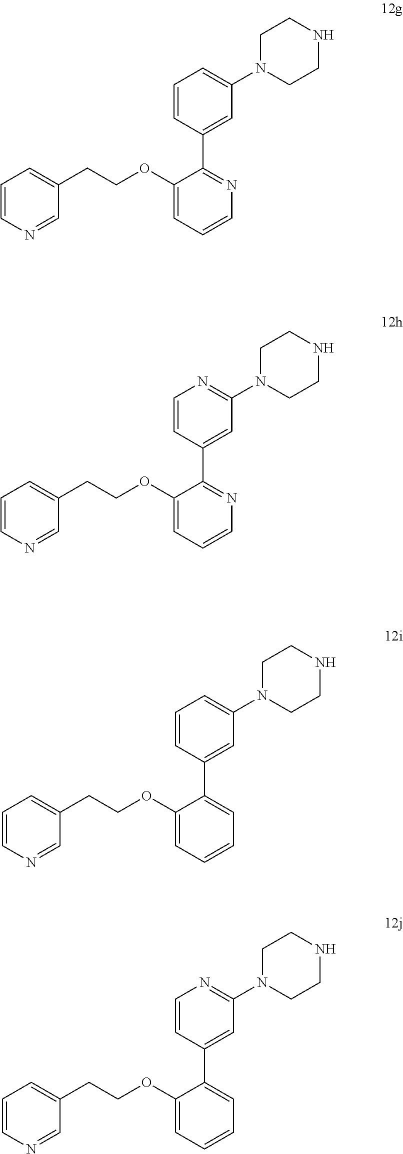 [SMILES: c1cncc(CCOc2cccnc2-c2cccc(N3CCNCC3)c2)c1 ; c1cncc(CCOc2cccnc2-c2ccnc(N3CCNCC3)c2)c1 ; c1cncc(CCOc2ccccc2-c2cccc(N3CCNCC3)c2)c1 ; c1cncc(CCOc2ccccc2-c2ccnc(N3CCNCC3)c2)c1]

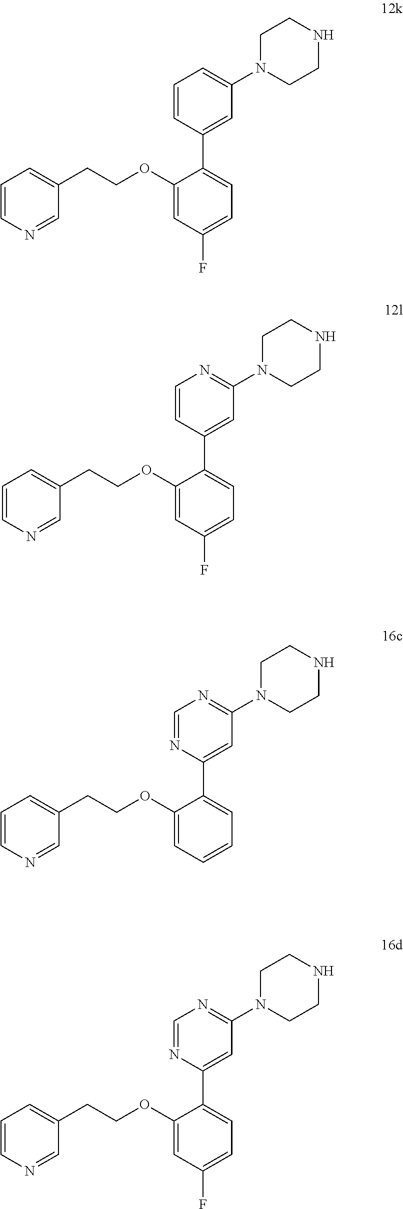 [SMILES: Fc1ccc(-c2cccc(N3CCNCC3)c2)c(OCCc2cccnc2)c1 ; Fc1ccc(-c2ccnc(N3CCNCC3)c2)c(OCCc2cccnc2)c1 ; c1cncc(CCOc2ccccc2-c2cc(N3CCNCC3)ncn2)c1 ; Fc1ccc(-c2cc(N3CCNCC3)ncn2)c(OCCc2cccnc2)c1]

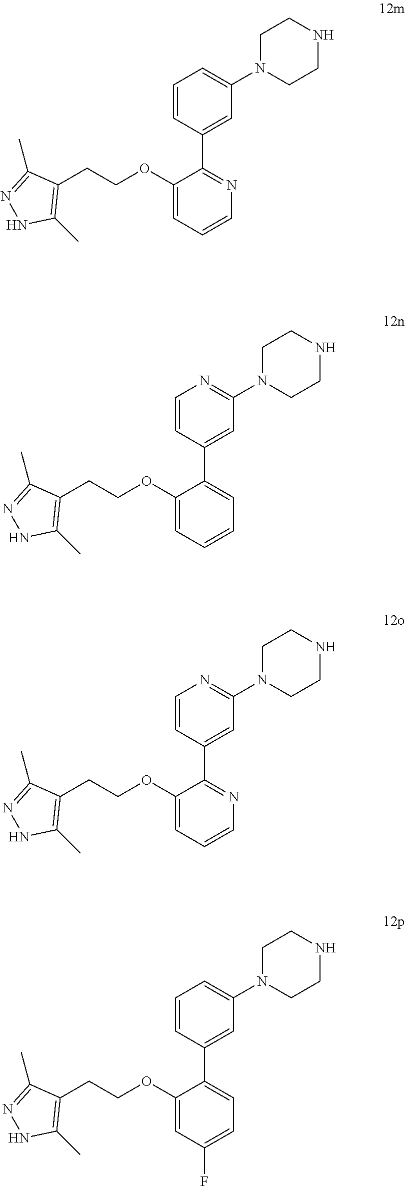 [SMILES: Cc1n[nH]c(C)c1CCOc1cccnc1-c1cccc(N2CCNCC2)c1 ; Cc1n[nH]c(C)c1CCOc1ccccc1-c1ccnc(N2CCNCC2)c1 ; Cc1n[nH]c(C)c1CCOc1cccnc1-c1ccnc(N2CCNCC2)c1 ; Cc1n[nH]c(C)c1CCOc1cc(F)ccc1-c1cccc(N2CCNCC2)c1]

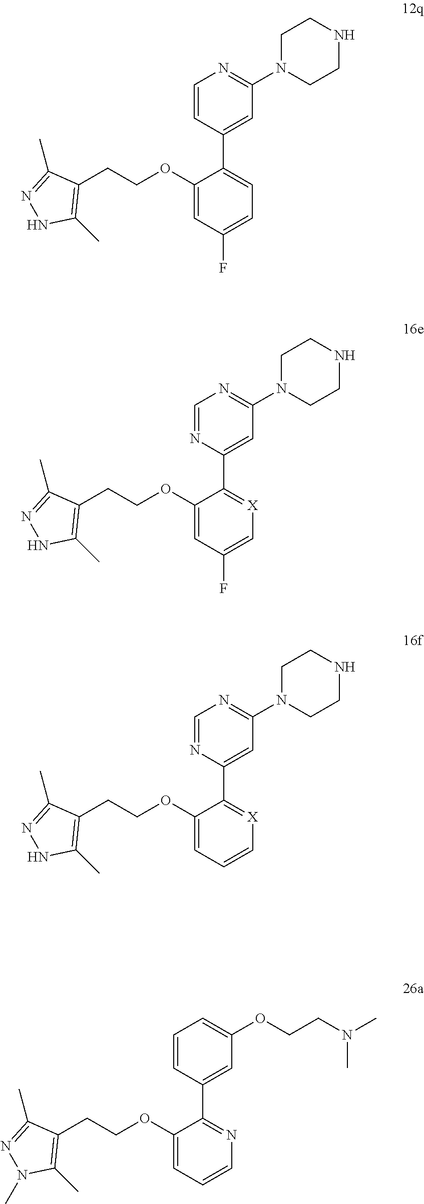

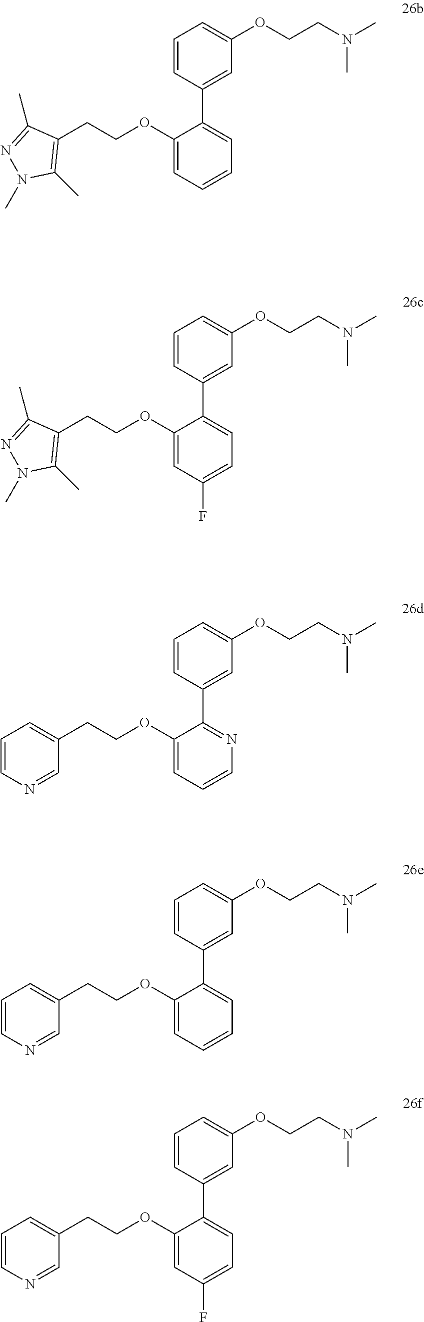 [SMILES: Cc1nn(C)c(C)c1CCOc1ccccc1-c1cccc(OCCN(C)C)c1 ; Cc1nn(C)c(C)c1CCOc1cc(F)ccc1-c1cccc(OCCN(C)C)c1 ; CN(C)CCOc1cccc(-c2ncccc2OCCc2cccnc2)c1 ; CN(C)CCOc1cccc(-c2ccccc2OCCc2cccnc2)c1 ; CN(C)CCOc1cccc(-c2ccc(F)cc2OCCc2cccnc2)c1]

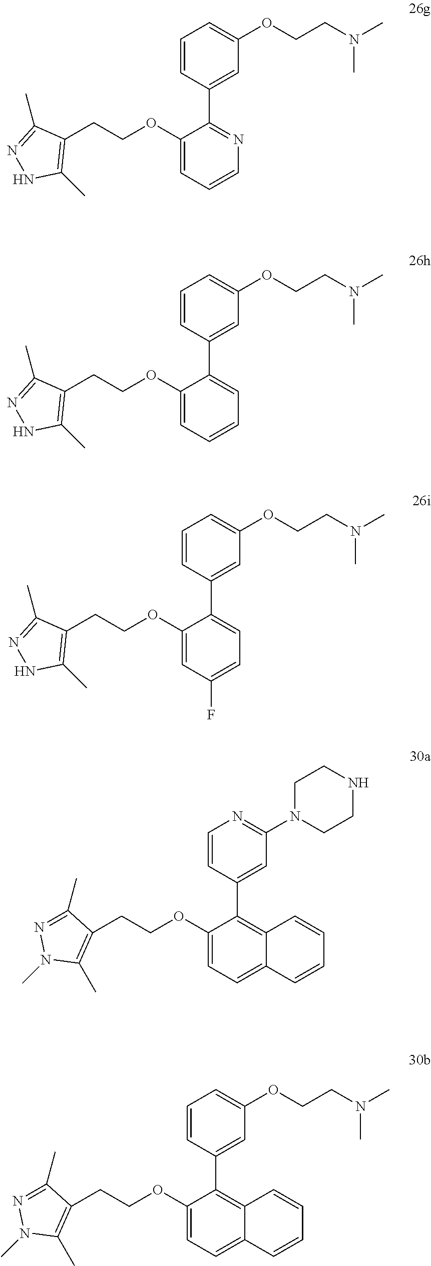

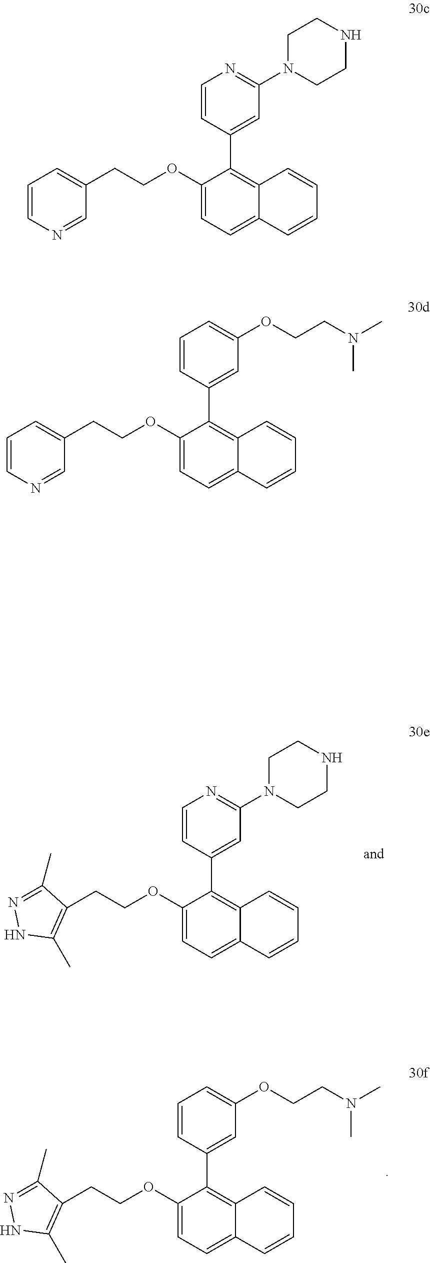 [SMILES: c1cncc(CCOc2ccc3ccccc3c2-c2ccnc(N3CCNCC3)c2)c1 ; CN(C)CCOc1cccc(-c2c(OCCc3cccnc3)ccc3ccccc23)c1 ; Cc1n[nH]c(C)c1CCOc1ccc2ccccc2c1-c1ccnc(N2CCNCC2)c1 ; Cc1n[nH]c(C)c1CCOc1ccc2ccccc2c1-c1cccc(OCCN(C)C)c1]

14. A method of treating, ameliorating, or preventing malaria in a subject in need thereof, the method comprising: administering to the subject a therapeutically effective amount of a compound of claim 1 and at least one pharmaceutically acceptable excipient or carrier.

15. The method of claim 14, wherein the administering is by a route selected from the group consisting of oral, buccal, transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

16. The method of claim 14, wherein the subject is a human.

17. The method of claim 14, wherein the subject is infected by Plasmodium falciparum or Plasmodium vivax.

18. The method of claim 17, wherein the Plasmodium vivax is in a dormant form (hypnozoites) in the subject.

19. A method of killing or inhibiting a Plasmodium falciparum or Plasmodium vivax parasite, the method comprising: contacting the parasite with an effective amount of the compound of claim 1 sufficient to kill the parasite or render the parasite incapable of causing malaria in a subject.

20. The method of claim 19, wherein the Plasmodium vivax is in a dormant form.

21-24. (canceled)
